SEQUENCE LISTING (34 sequences)

SEQ ID NO: 1 - DNA, 975 bp, Artificial Sequence
  Description of Artificial Sequence Synthetic polynucleotide
gtctgctccagggacttcaccccgcccaccgtgaagatcttacagtcgtcctgcgacggcggcgggcacttccccccgaccatccagctcctgtgcctcgtctctgggtacaccccagggactatcaacatcacctggctggaggacgggcaggtcatggacgtggacttgtccaccgcctctaccacgcaggagggtgagctggcctccacacaaagcgagctcaccctcagccagaagcactggctgtcagaccgcacctacacctgccaggtcacctatcaaggtcacacctttgaggacagcaccaagaagtgtgcagattccaacccgagaggggtgagcgcctacctaagccggcccagcccgttcgacctgttcatccgcaagtcgcccacgatcacctgtctggtggtggacctggcacccagcaaggggaccgtgaacctgacctggtcccgggccagtgggaagcctgtgaaccactccaccagaaaggaggagaagcagcgcaatggcacgttaaccgtcacgtccaccctgccggtgggcacccgagactggatcgagggggagacctaccagtgcagggtgacccacccccacctgcccagggccctcatgcggtccacgaccaagaccagcggcccgcgtgctgccccggaagtctatgcgtttgcgacgccggagtggccggggagccgggacaagcgcaccctcgcctgcctgatccagaacttcatgcctgaggacatctcggtgcagtggctgcacaacgaggtgcagctcccggacgcccggcacagcacgacgcagccccgcaagaccaagggctccggcttcttcgtcttcagccgcctggaggtgaccagggccgaatgggagcagaaagatgagttcatctgccgtgcagtccatgaggcagcgagcccctcacagaccgtccagcgagcggtgtctgtaaatcccggtaaa

SEQ ID NO: 2 - DNA, 1008 bp, Artificial Sequence
  Description of Artificial Sequence Synthetic polynucleotide
atggttagatctgttcgacctgtcaacatcactgagcccaccttggagctactccattcatcctgcgaccccaatgcattccactccaccatccagctgtactgcttcatttatggccacatcctaaatgatgtctctgtcagctggctaatggacgatcgggagataactgatacacttgcacaaactgttctaatcaaggaggaaggcaaactagcctctacctgcagtaaactcaacatcactgagcagcaatggatgtctgaaagcaccttcacctgcaaggtcacctcccaaggcgtagactatttggcccacactcggagatgcccagatcatgagccacggggtgtgattacctacctgatcccacccagccccctggacctgtatcaaaacggtgctcccaagcttacctgtctggtggtggacctggaaagcgagaagaatgtcaatgtgacgtggaaccaagagaagaagacttcagtctcagcatcccagtggtacactaagcaccacaataacgccacaactagtatcacctccatcctgcctgtagttgccaaggactggattgaaggctacggctatcagtgcatagtggaccaccctgattttcccaagcccattgtgcgttccatcaccaagaccccaggccagcgctcagcccccgaggtatatgtgttcccaccaccagaggaggagagcgaggacaaacgcacactcacctgtttgatccagaacttcttccctgaggatatctctgtgcagtggctgggggatggcaaactgatctcaaacagccagcacagtaccacaacacccctgaaatccaatggctccaatcaaggcttcttcatcttcagtcgcctagaggtcgccaagacactctggacacagagaaaacagttcacctgccaagtgatccatgaggcacttcagaaacccaggaaactggagaaaacaatatccacaagccttggtaacacctccctccgtccctcctag

SEQ ID NO: 3 - protein, 356 aa, Artificial Sequence
  Description of Artificial Sequence Synthetic polypeptide
TVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 4 - protein, 356 aa, Artificial Sequence
  Description of Artificial Sequence Synthetic polypeptide
TVSGAWAKQMFTCRVAHTPSSTDWVDNKTFSVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGALTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGK

SEQ ID NO: 5 - protein, 335 aa, Artificial Sequence
  Description of Artificial Sequence Synthetic polypeptide
MVRSVRPVNITEPTLELLHSSCDPNAFHSTIQLYCFIYGHILNDVSVSWLMDDREITDTLAQTVLIKEEGKLASTCSKLNITEQQWMSESTFTCKVTSQGVDYLAHTRRCPDHEPRGVITYLIPPSPLDLYQNGAPKLTCLVVDLESEKNVNVTWNQEKKTSVSASQWYTKHHNNATTSITSILPVVAKDWIEGYGYQCIVDHPDFPKPIVRSITKTPGQRSAPEVYVFPPPEEESEDKRTLTCLIQNFFPEDISVQWLGDGKLISNSQHSTTTPLKSNGSNQGFFIFSRLEVAKTLWTQRKQFTCQVIHEALQKPRKLEKTISTSLGNTSLRPS

SEQ ID NO: 6 - protein, 331 aa, Artificial Sequence
  Description of Artificial Sequence Synthetic polypeptide
VRPVNITEPTLELLHSSCDPNAFHSTIQLYCFIYGHILNDVSVSWLMDDREITDTLAQTVLIKEEGKLASTCSKLNITEQQWMSESTFTCKVTSQGVDYLAHTRRCPDHEPRGVITYLIPPSPLDLYQNGAPKLTCLVVDLESEKNVNVTWNQEKKTSVSASQWYTKHHNNATTSITSILPVVAKDWIEGYGYQCIVDHPDFPKPIVRSITKTPGQRSAPEVYVFPPPEEESEDKRTLTCLIQNFFPEDISVQWLGDGKLISNSQHSTTTPLKSNGSNQGFFIFSRLEVAKTLWTQRKQFTCQVIHEALQKPRKLEKTISTSLGNTSLRPS

SEQ ID NO: 7 - DNA, 682 bp, Artificial Sequence
  Description of Artificial Sequence Synthetic polynucleotide
tgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 8 - protein, 227 aa, Artificial Sequence
  Description of Artificial Sequence Synthetic polypeptide
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 231 aa, Artificial Sequence
  Description of Artificial Sequence Synthetic polypeptide
MVRSDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 233 aa, Artificial Sequence
  Description of Artificial Sequence Synthetic polypeptide
MVRSDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGS

SEQ ID NO: 11 - DNA, 1740 bp, Artificial Sequence
  Description of Artificial Sequence Synthetic polynucleotide
atggttagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtggaacacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccttcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaaggatccggcggcggaggctccggcggcggaggctccggcggcggaggctccgttcgacctgtcaacatcactgagcccaccttggagctactccattcatcctgcgaccccaatgcattccactccaccatccagctgtactgcttcatttatggccacatcctaaatgatgtctctgtcagctggctaatggacgatcgggagataactgatacacttgcacaaactgttctaatcaaggaggaaggcaaactagcctctacctgcagtaaactcaacatcactgagcagcaatggatgtctgaaagcaccttcacctgcaaggtcacctcccaaggcgtagactatttggcccacactcggagatgcccagatcatgagccacggggtgtgattacctacctgatcccacccagccccctggacctgtatcaaaacggtgctcccaagcttacctgtctggtggtggacctggaaagcgagaagaatgtcaatgtgacgtggaaccaagagaagaagacttcagtctcagcatcccagtggtacactaagcaccacaataacgccacaactagtatcacctccatcctgcctgtagttgccaaggactggattgaaggctacggctatcagtgcatagtggaccaccctgattttcccaagcccattgtgcgttccatcaccaagaccccaggccagcgctcagcccccgaggtatatgtgttcccaccaccagaggaggagagcgaggacaaacgcacactcacctgtttgatccagaacttcttccctgaggatatctctgtgcagtggctgggggatggcaaactgatctcaaacagccagcacagtaccacaacacccctgaaatccaatggctccaatcaaggcttcttcatcttcagtcgcctagaggtcgccaagacactctggacacagagaaaacagttcacctgccaagtgatccatgaggcacttcagaaacccaggaaactggagaaaacaatatccacaagccttggtaacacctccctccgtccctcctag

SEQ ID NO: 12 - protein, 579 aa, Artificial Sequence
  Description of Artificial Sequence Synthetic polypeptide
MVRSDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGGGSGGGGSGGGGSVRPVNITEPTLELLHSSCDPNAFHSTIQLYCFIYGHILNDVSVSWLMDDREITDTLAQTVLIKEEGKLASTCSKLNITEQQWMSESTFTCKVTSQGVDYLAHTRRCPDHEPRGVITYLIPPSPLDLYQNGAPKLTCLVVDLESEKNVNVTWNQEKKTSVSASQWYTKHHNNATTSITSILPVVAKDWIEGYGYQCIVDHPDFPKPIVRSITKTPGQRSAPEVYVFPPPEEESEDKRTLTCLIQNFFPEDISVQWLGDGKLISNSQHSTTTPLKSNGSNQGFFIFSRLEVAKTLWTQRKQFTCQVIHEALQKPRKLEKTISTSLGNTSLRPS

SEQ ID NO: 13 - protein, 5 aa, Artificial Sequence
  Description of Artificial Sequence Synthetic peptide
GGGGS

SEQ ID NO: 14 - protein, 15 aa, Artificial Sequence
  Description of Artificial Sequence Synthetic peptide
GGGGSGGGGSGGGGS

SEQ ID NO: 15 - protein, 5 aa, Artificial Sequence
  Description of Artificial Sequence Synthetic peptide
GGSSG

SEQ ID NO: 16 - protein, 5 aa, Artificial Sequence
  Description of Artificial Sequence Synthetic peptide
GGGGG

SEQ ID NO: 17 - protein, 8 aa, Artificial Sequence
  Description of Artificial Sequence Synthetic peptide
GSGSGSGS

SEQ ID NO: 18 - protein, 4 aa, Artificial Sequence
  Description of Artificial Sequence Synthetic peptide
GGGS

SEQ ID NO: 19 - protein, 20 aa, Artificial Sequence
  Description of Artificial Sequence Synthetic Peptide
  MISC_FEATURE (1)..(20): This sequence may encompass 1-10 “Gly Ser” repeating units
GSGSGSGSGSGSGSGSGSGS

SEQ ID NO: 20 - DNA, 5166 bp, Artificial Sequence
  Description of Artificial Sequence Synthetic polynucleotide
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggcgaaggagggccaccatgtacaggatgcaactcctgtcttgcattgcactaagtcttgcacttgtcacgaattcgatatctcgagtggtctgctccagggacttcaccccgcccaccgtgaagatcttacagtcgtcctgcgacggcggcgggcacttccccccgaccatccagctcctgtgcctcgtctctgggtacaccccagggactatcaacatcacctggctggaggacgggcaggtcatggacgtggacttgtccaccgcctctaccacgcaggagggtgagctggcctccacacaaagcgagctcaccctcagccagaagcactggctgtcagaccgcacctacacctgccaggtcacctatcaaggtcacacctttgaggacagcaccaagaagtgtgcagattccaacccgagaggggtgagcgcctacctaagccggcccagcccgttcgacctgttcatccgcaagtcgcccacgatcacctgtctggtggtggacctggcacccagcaaggggaccgtgaacctgacctggtcccgggccagtgggaagcctgtgaaccactccaccagaaaggaggagaagcagcgcaatggcacgttaaccgtcacgtccaccctgccggtgggcacccgagactggatcgagggggagacctaccagtgcagggtgacccacccccacctgcccagggccctcatgcggtccacgaccaagaccagcggcccgcgtgctgccccggaagtctatgcgtttgcgacgccggagtggccggggagccgggacaagcgcaccctcgcctgcctgatccagaacttcatgcctgaggacatctcggtgcagtggctgcacaacgaggtgcagctcccggacgcccggcacagcacgacgcagccccgcaagaccaagggctccggcttcttcgtcttcagccgcctggaggtgaccagggccgaatgggagcagaaagatgagttcatctgccgtgcagtccatgaggcagcgagcccctcacagaccgtccagcgagcggtgtctgtaaatcccggtaaaagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaatgagtgctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 21 - protein, 578 aa, Artificial Sequence
  Description of Artificial Sequence Synthetic polypeptide
MYRMQLLSCIALSLALVTNSISRVVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - DNA, 5104 bp, Artificial Sequence
  Description of Artificial Sequence Synthetic polynucleotide
ggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctacctgagatcaccggtgaattcgatatctcgagcaccatggtctgctccagggacttcaccccgcccaccgtgaagatcttacagtcgtcctgcgacggcggcgggcacttccccccgaccatccagctcctgtgcctcgtctctgggtacaccccagggactatcaacatcacctggctggaggacgggcaggtcatggacgtggacttgtccaccgcctctaccacgcaggagggtgagctggcctccacacaaagcgagctcaccctcagccagaagcactggctgtcagaccgcacctacacctgccaggtcacctatcaaggtcacacctttgaggacagcaccaagaagtgtgcagattccaacccgagaggggtgagcgcctacctaagccggcccagcccgttcgacctgttcatccgcaagtcgcccacgatcacctgtctggtggtggacctggcacccagcaaggggaccgtgaacctgacctggtcccgggccagtgggaagcctgtgaaccactccaccagaaaggaggagaagcagcgcaatggcacgttaaccgtcacgtccaccctgccggtgggcacccgagactggatcgagggggagacctaccagtgcagggtgacccacccccacctgcccagggccctcatgcggtccacgaccaagaccagcggcccgcgtgctgccccggaagtctatgcgtttgcgacgccggagtggccggggagccgggacaagcgcaccctcgcctgcctgatccagaacttcatgcctgaggacatctcggtgcagtggctgcacaacgaggtgcagctcccggacgcccggcacagcacgacgcagccccgcaagaccaagggctccggcttcttcgtcttcagccgcctggaggtgaccagggccgaatgggagcagaaagatgagttcatctgccgtgcagtccatgaggcagcgagcccctcacagaccgtccagcgagcggtgtctgtaaatcccggtaaaagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccactccccgaggagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaatgagtgctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaattaattctaaaatacagcatagcaaaactttaacctccaaatcaagcctctacttgaatccttttctgagggatgaataaggcataggcatcaggggctgttgccaatgtgcattagctgtttgcagcctcaccttctttcatggagtttaagatatagtgtattttcccaaggtttgaactagctcttcatttctttatgttttaaatgcactgacctcccacattccctttttagtaaaatattcagaaataatttaaatacatcattgcaatgaaaataaatgttttttattaggcagaatccagatgctcaaggcccttcataatatcccccagtttagtagttggacttagggaacaaaggaacctttaatagaaattggacagcaagaaagcgagcttctagcttatcctcagtcctgctcctctgccacaaagtgcacgcagttgccggccgggtcgcgcagggcgaactcccgcccccacggctgctcgccgatctcggtcatggccggcccggaggcgtcccggaagttcgtggacacgacctccgaccactcggcgtacagctcgtccaggccgcgcacccacacccaggccagggtgttgtccggcaccacctggtcctggaccgcgctgatgaacagggtcacgtcgtcccggaccacaccggcgaagtcgtcctccacgaagtcccgggagaacccgagccggtcggtccagaactcgaccgctccggcgacgtcgcgcgcggtgagcaccggaacggcactggtcaacttggccatgatggctcctcctgtcaggagaggaaagagaagaaggttagtacaattgctatagtgagttgtattatactatgcagatatactatgccaatgattaattgtcaaactagggctgcagggttcatagtgccacttttcctgcactgccccatctcctgcccaccctttcccaggcatagacagtcagtgacttaccaaactcacaggagggagaaggcagaagcttgagacagacccgcgggaccgccgaactgcgaggggacgtggctagggcggcttcttttatggtgcgccggccctcggaggcagggcgctcggggaggcctagcggccaatctgcggtggcaggaggcggggccgaaggccgtgcctgaccaatccggagcacataggagtctcagccccccgccccaaagcaaggggaagtcacgcgcctgtagcgccagcgtgttgtgaaatgggggcttgggggggttggggccctgactagtcaaaacaaactcccattgacgtcaatggggtggagacttggaaatccccgtgagtcaaaccgctatccacgcccattgatgtactgccaaaaccgcatcatcatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcctgcaggttaattaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatggctagttaattaacatttaaatcagcggccgcaataaaatatctttattttcattacatctgtgtgttggttttttgtgtgaatcgtaactaacatacgctctccatcaaaacaaaacgaaacaaaacaaactagcaaaataggctgtccccagtgcaagtgcaggtgccagaacatttctctatcgaa

SEQ ID NO: 23 - protein, 555 aa, Artificial Sequence
  Description of Artificial Sequence Synthetic polypeptide
MVCSRDFTPPTVKILQSSCDGGGHFPPTIQLLCLVSGYTPGTINITWLEDGQVMDVDLSTASTTQEGELASTQSELTLSQKHWLSDRTYTCQVTYQGHTFEDSTKKCADSNPRGVSAYLSRPSPFDLFIRKSPTITCLVVDLAPSKGTVNLTWSRASGKPVNHSTRKEEKQRNGTLTVTSTLPVGTRDWIEGETYQCRVTHPHLPRALMRSTTKTSGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPGKRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPLPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - DNA, 8745 bp, Artificial Sequence
  Description of Artificial Sequence Synthetic polynucleotide
cctgcagggcctgaaataacctctgaaagaggaacttggttaggtaccttctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccactagtggagccgagagtaattcatacaaaaggagggatcgccttcgcaaggggagagcccagggaccgtccctaaattctcacagacccaaatccctgtagccgccccacgacagcgcgaggagcatgcgctcagggctgagcgcggggagagcagagcacacaagctcatagaccctggtcgtgggggggaggaccggggagctggcgcggggcaaactgggaaagcggtgtcgtgtgctggctccgccctcttcccgagggtgggggagaacggtatataagtgcggcagtcgccttggacgttctttttcgcaacgggtttgccgtcagaacgcaggtgaggggcgggtgtggcttccgcgggccgccgagctggaggtcctgctccgagcgggccgggccccgctgtcgtcggcggggattagctgcgagcattcccgcttcgagttgcgggcggcgcgggaggcagagtgcgaggcctagcggcaaccccgtagcctcgcctcgtgtccggcttgaggcctagcgtggtgtccgcgccgccgccgcgtgctactccggccgcactctggtcttttttttttttgttgttgttgccctgctgccttcgattgccgttcagcaataggggctaacaaagggagggtgcggggcttgctcgcccggagcccggagaggtcatggttggggaggaatggagggacaggagtggcggctggggcccgcccgccttcggagcacatgtccgacgccacctggatggggcgaggcctggggtttttcccgaagcaaccaggctggggttagcgtgccgaggccatgtggccccagcacccggcacgatctggcttggcggcgccgcgttgccctgcctccctaactagggtgaggccatcccgtccggcaccagttgcgtgcgtggaaagatggccgctcccgggccctgttgcaaggagctcaaaatggaggacgcggcagcccggtggagcgggcgggtgagtcacccacacaaaggaagagggcctggtccctcaccggctgctgcttcctgtgaccccgtggtcctatcggccgcaatagtcacctcgggcttttgagcacggctagtcgcggcggggggaggggatgtaatggcgttggagtttgttcacatttggtgggtggagactagtcaggccagcctggcgctggaagtcatttttggaatttgtccccttgagttttgagcggagctaattctcgggcttcttagcggttcaaaggtatcttttaaacccttttttaggtgttgtgaaaaccaccgctaattcaaagcaaccggtcaccatggttagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtggaacacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccttcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaaggatccggcggcggaggctccggcggcggaggctccggcggcggaggctccgttcgacctgtcaacatcactgagcccaccttggagctactccattcatcctgcgaccccaatgcattccactccaccatccagctgtactgcttcatttatggccacatcctaaatgatgtctctgtcagctggctaatggacgatcgggagataactgatacacttgcacaaactgttctaatcaaggaggaaggcaaactagcctctacctgcagtaaactcaacatcactgagcagcaatggatgtctgaaagcaccttcacctgcaaggtcacctcccaaggcgtagactatttggcccacactcggagatgcccagatcatgagccacggggtgtgattacctacctgatcccacccagccccctggacctgtatcaaaacggtgctcccaagcttacctgtctggtggtggacctggaaagcgagaagaatgtcaatgtgacgtggaaccaagagaagaagacttcagtctcagcatcccagtggtacactaagcaccacaataacgccacaactagtatcacctccatcctgcctgtagttgccaaggactggattgaaggctacggctatcagtgcatagtggaccaccctgattttcccaagcccattgtgcgttccatcaccaagaccccaggccagcgctcagcccccgaggtatatgtgttcccaccaccagaggaggagagcgaggacaaacgcacactcacctgtttgatccagaacttcttccctgaggatatctctgtgcagtggctgggggatggcaaactgatctcaaacagccagcacagtaccacaacacccctgaaatccaatggctccaatcaaggcttcttcatcttcagtcgcctagaggtcgccaagacactctggacacagagaaaacagttcacctgccaagtgatccatgaggcacttcagaaacccaggaaactggagaaaacaatatccacaagccttggtaacacctccctccgtccctccgaaaatctttattttcaaggtcatcatcatcatcatcatcattaggctagctggccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggaaatgttaattaactagccatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttcttaattaacctgcaggcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgatgatgcggttttggcagtacatcaatgggcgtggatagcggtttgactcacggggatttccaagtctccaccccattgacgtcaatgggagtttgttttgactagtggagccgagagtaattcatacaaaaggagggatcgccttcgcaaggggagagcccagggaccgtccctaaattctcacagacccaaatccctgtagccgccccacgacagcgcgaggagcatgcgcccagggctgagcgcgggtagatcagagcacacaagctcacagtccccggcggtggggggaggggcgcgctgagcgggggccagggagctggcgcggggcaaactgggaaagtggtgtcgtgtgctggctccgccctcttcccgagggtgggggagaacggtatataagtgcggtagtcgccttggacgttctttttcgcaacgggtttgccgtcagaacgcaggtgagtggcgggtgtggcttccgcgggccccggagctggagccctgctctgagcgggccgggctgatatgcgagtgtcgtccgcagggtttagctgtgagcattcccacttcgagtggcgggcggtgcgggggtgagagtgcgaggcctagcggcaaccccgtagcctcgcctcgtgtccggcttgaggcctagcgtggtgtccgccgccgcgtgccactccggccgcactatgcgttttttgtccttgctgccctcgattgccttccagcagcatgggctaacaaagggagggtgtggggctcactcttaaggagcccatgaagcttacgttggataggaatggaagggcaggaggggcgactggggcccgcccgccttcggagcacatgtccgacgccacctggatggggcgaggcctgtggctttccgaagcaatcgggcgtgagtttagcctacctgggccatgtggccctagcactgggcacggtctggcctggcggtgccgcgttcccttgcctcccaacaagggtgaggccgtcccgcccggcaccagttgcttgcgcggaaagatggccgctcccggggccctgttgcaaggagctcaaaatggaggacgcggcagcccggtggagcgggcgggtgagtcacccacacaaaggaagagggccttgcccctcgccggccgctgcttcctgtgaccccgtggtctatcggccgcatagtcacctcgggcttctcttgagcaccgctcgtcgcggcggggggaggggatctaatggcgttggagtttgttcacatttggtgggtggagactagtcaggccagcctggcgctggaagtcattcttggaatttgcccctttgagtttggagcgaggctaattctcaagcctcttagcggttcaaaggtattttctaaacccgtttccaggtgttgtgaaagccaccgctaattcaaagcaatccggagtatacatggttagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtggaacacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccttcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaatgacctaggagcaggtttccccaatgacacaaaacgtgcaacttgaaactccgcctggtctttccaggtctagaggggtaacactttgtactgcgtttggctccacgctcgatccactggcgagtgttagtaacagcactgttgcttcgtagcggagcatgacggccgtgggaactcctccttggtaacaaggacccacggggccaaaagccacgcccacacgggcccgtcatgtgtgcaaccccagcacggcgactttactgcgaaacccactttaaagtgacattgaaactggtacccacacactggtgacaggctaaggatgcccttcaggtaccccgaggtaacacgcgacactcgggatctgagaaggggactggggcttctataaaagcgctcggtttaaaaagcttctatgcctgaataggtgaccggaggtcggcacctttcctttgcaattactgaccctatgaatacactgactgtttgacaattaatcatcggcatagtatatcggcatagtataatacgactcactataggagggccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacacatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgaattcgctaggattatccctaatacctgccaccccactcttaatcagtggtggaagaacggtctcagaactgtttgtttcaattggccatttaagtttagtagtaaaagactggttaatgataacaatgcatcgtaaaaccttcagaaggaaaggagaatgttttgtggaccactttggttttcttttttgcgtgtggcagttttaagttattagtttttaaaatcagtactttttaatggaaacaacttgaccaaaaatttgtcacagaattttgagacccattaaaaaagttaaatgagaaacctgtgtgttcctttggtcaacaccgagacatttaggtgaaagacatctaattctggttttacgaatctggaaacttcttgaaaatgtaattcttgagttaacacttctgggtggagaatagggttgttttccccccacataattggaaggggaaggaatatcatttaaagctatgggagggttgctttgattacaacactggagagaaatgcagcatgttgctgattgcctgtcactaaaacaggccaaaaactgagtccttgggttgcatagaaagctg

SEQ ID NO: 25 - protein, 593 aa, Artificial Sequence
  Description of Artificial Sequence Synthetic polypeptide
MVRSDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGGGSGGGGSGGGGSVRPVNITEPTLELLHSSCDPNAFHSTIQLYCFIYGHILNDVSVSWLMDDREITDTLAQTVLIKEEGKLASTCSKLNITEQQWMSESTFTCKVTSQGVDYLAHTRRCPDHEPRGVITYLIPPSPLDLYQNGAPKLTCLVVDLESEKNVNVTWNQEKKTSVSASQWYTKHHNNATTSITSILPVVAKDWIEGYGYQCIVDHPDFPKPIVRSITKTPGQRSAPEVYVFPPPEEESEDKRTLTCLIQNFFPEDISVQWLGDGKLISNSQHSTTTPLKSNGSNQGFFIFSRLEVAKTLWTQRKQFTCQVIHEALQKPRKLEKTISTSLGNTSLRPSENLYFQGHHHHHHH

SEQ ID NO: 26 - protein, 231 aa, Artificial Sequence
  Description of Artificial Sequence Synthetic polypeptide
MVRSDKTHTCPPCPAPELLGGPSVFLFPPKPKDTLMISRTPEVTCVVVDVEHEDPEVKFNWYVDGVEVHNAKTKPREEQYNSTYRVVSVLTVLHQDWLNGKEYKCKVSNKAFPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 27 - DNA, 1740 bp, Artificial Sequence
  Description of Artificial Sequence Synthetic polynucleotide
atggttagatctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgcgaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcacgaggctctgcacaaccactacacgcagaagagcctctccctgtctccgggtaaaggatccggcggcggaggctccggcggcggaggctccggcggcggaggctccgttcgacctgtcaacatcactgagcccaccttggagctactccattcatcctgcgaccccaatgcattccactccaccatccagctgtactgcttcatttatggccacatcctaaatgatgtctctgtcagctggctaatggacgatcgggagataactgatacacttgcacaaactgttctaatcaaggaggaaggcaaactagcctctacctgcagtaaactcaacatcactgagcagcaatggatgtctgaaagcaccttcacctgcaaggtcacctcccaaggcgtagactatttggcccacactcggagatgcccagatcatgagccacggggtgtgattacctacctgatcccacccagccccctggacctgtatcaaaacggtgctcccaagcttacctgtctggtggtggacctggaaagcgagaagaatgtcaatgtgacgtggaaccaagagaagaagacttcagtctcagcatcccagtggtacactaagcaccacaataacgccacaactagtatcacctccatcctgcctgtagttgccaaggactggattgaaggctacggctatcagtgcatagtggaccaccctgattttcccaagcccattgtgcgttccatcaccaagaccccaggccagcgctcagcccccgaggtatatgtgttcccaccaccagaggaggagagcgaggacaaacgcacactcacctgtttgatccagaacttcttccctgaggatatctctgtgcagtggctgggggatggcaaactgatctcaaacagccagcacagtaccacaacacccctgaaatccaatggctccaatcaaggcttcttcatcttcagtcgcctagaggtcgccaagacactctggacacagagaaaacagttcacctgccaagtgatccatgaggcacttcagaaacccaggaaactggagaaaacaatatccacaagccttggtaacacctccctccgtccctcctag

SEQ ID NO: 28 - protein, 579 aa, Artificial Sequence
  Description of Artificial Sequence Synthetic polypeptide
MVRSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIAKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGSGGGGSGGGGSGGGGSVRPVNITEPTLELLHSSCDPNAFHSTIQLYCFIYGHILNDVSVSWLMDDREITDTLAQTVLIKEEGKLASTCSKLNITEQQWMSESTFTCKVTSQGVDYLAHTRRCPDHEPRGVITYLIPPSPLDLYQNGAPKLTCLVVDLESEKNVNVTWNQEKKTSVSASQWYTKHHNNATTSITSILPVVAKDWIEGYGYQCIVDHPDFPKPIVRSITKTPGQRSAPEVYVFPPPEEESEDKRTLTCLIQNFFPEDISVQWLGDGKLISNSQHSTTTPLKSNGSNQGFFIFSRLEVAKTLWTQRKQFTCQVIHEALQKPRKLEKTISTSLGNTSLRPS

SEQ ID NO: 29 - protein, 345 aa, Artificial Sequence
  Description of Artificial Sequence Synthetic polypeptide
VRPVNITEPTLELLHSSCDPNAFHSTIQLYCFIYGHILNDVSVSWLMDDREITDTLAQTVLIKEEGKLASTCSKLNITEQQWMSESTFTCKVTSQGVDYLAHTRRCPDHEPRGVITYLIPPSPLDLYQNGAPKLTCLVVDLESEKNVNVTWNQEKKTSVSASQWYTKHHNNATTSITSILPVVAKDWIEGYGYQCIVDHPDFPKPIVRSITKTPGQRSAPEVYVFPPPEEESEDKRTLTCLIQNFFPEDISVQWLGDGKLISNSQHSTTTPLKSNGSNQGFFIFSRLEVAKTLWTQRKQFTCQVIHEALQKPRKLEKTISTSLGNTSLRPSENLYFQGHHHHHHH

SEQ ID NO: 30 - protein, 4 aa, Artificial Sequence
  Description of Artificial Sequence Synthetic peptide
GAGA

SEQ ID NO: 31 - protein, 100 aa, Artificial Sequence
  Description of Artificial Sequence Synthetic Polypeptide
  MISC_FEATURE (1)..(100): This sequence may encompass 1-12, 15, or 20 “Gly Gly Gly Gly Ser” repeating units
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 32 - protein, 6 aa, Artificial Sequence
  Description of Artificial Sequence Synthetic 6xHis tag
HHHHHH

SEQ ID NO: 33 - protein, 20 aa, Artificial Sequence
  Description of Artificial Sequence Synthetic peptide
  MISC_FEATURE (1)..(20): This sequence may encompass 1-12, 15 or 20 residues
GGGGGGGGGGGGGGGGGGGG

SEQ ID NO: 34 - protein, 20 aa, Artificial Sequence
  Description of Artificial Sequence Synthetic peptide
  MISC_FEATURE (1)..(10): This region may encompass 1-10 residues
  MISC_FEATURE (11)..(20): This region may encompass 1-10 residues
GGGGGGGGGGSSSSSSSSSS

(19) United States
(12) Patent Application Publication
(10) Pub. No.: US 2021/0380651 A1
(43) Pub. Date: Dec. 9, 2021

(54) COMPOSITIONS FOR DISEASE TREATMENT

(71) Applicant: Siolta Therapeutics, Inc., San Carlos, CA (US)

(72) Inventors: Nikole E. KIMES, San Francisco, CA (US); Benjamin FIEBIGER, San Francisco, CA (US); Ricardo VALLADARES, Brookhaven, GA (US)

(21) Appl. No.: 17/407,111

(22) Filed: Aug. 19, 2021

Related U.S. Application Data
(63) Continuation of application No. PCT/US2020/019124, filed on Feb. 20, 2020.
(60) Provisional application No. 62/808,140, filed on Feb. 20, 2019.

(51) Int. Cl. C07K 14/47

(52) U.S. Cl. CPC C07K 14/47; C07K 2317/52; A61K 38/00; C07K 2317/92; C07K 2319/30

(57) ABSTRACT

The present disclosure provides methods and compositions comprising chimeric binding agents such as those comprising an Fc epsilon binding region and an Fc gamma binding region, and which may bind two or more of the receptors. The herein described methods and compositions may be used for treatment and prevention of various diseases and conditions, particularly IgE-mediated allergic and inflammatory diseases.

### CROSS-REFERENCE

This application is a continuation of International Patent Application No. PCT/US2020/019124, filed Feb. 20, 2020, which claims priority to and the benefit of U.S. Provisional Application No. 62/808,140, filed Feb. 20, 2019, the entire contents of each of which application is incorporated herein by reference in its entirety.

### SEQUENCE LISTING

The instant application contains a Sequence Listing which has been submitted electronically in ASCII format and is hereby incorporated by reference in its entirety. The ASCII copy, created on Mar. 25, 2020, is named 53206-705_301_SL and is 87,909 bytes in size.

### BACKGROUND

Allergic diseases may be inflammatory disorders that involve many types of cells and factors, including allergens, immunoglobulin molecules, mast cells, basophils, cytokines and soluble mediators. Millions of people worldwide suffer from allergic diseases, such as allergic rhinitis, allergic asthma, allergic conjunctivitis, eczema, food allergies and drug allergies.

Current treatment options include full-length antibodies, such as omalizumab, and present several drawbacks, including frequent administration (e.g., every 2-4 weeks), immunogenicity, and immune complex formation. Moreover, nearly 100% of Fc receptors (FcRs), specifically FcεRs and FcεRIs, may have to be occupied by an anti-FcεRI binding agent in order to provide durable and measurable responses in vivo. It has been shown that even trace amounts of antigen-specific IgE bound to FcεRIs can cause cellular degranulation of, e.g., mast cells, and lead to significant allergic reactions. Thus, there is an unmet clinical need for new treatment and prevention options for diseases, particularly inflammatory diseases and allergic diseases.

### BRIEF SUMMARY

The present disclosure provides methods and compositions comprising multivalent chimeric binding agents capable of simultaneously binding Fc receptors, such as an FCC receptor (FcεR) and an Fcγ receptor (FcγR). Such compositions may be used to treat diseases, such as allergic diseases.

In various instances, the present disclosure provides a chimeric binding agent, wherein the chimeric binding agent comprises: (i) an IgE-Fc region; and (ii) a plurality of IgG-Fc regions, wherein the IgE-Fc region binds to an Fc epsilon (Fcε) receptor and the plurality of IgG-Fc regions binds to an Fc gamma (Fcγ) receptor, thereby inhibiting the Fcε receptor while activating the Fcγ receptor. In some aspects, inhibiting the Fcε receptor and activating the Fcγ receptor occurs substantially simultaneously. In some aspects, the FcεR is an FcεRI. In some aspects, the FcγR is an FcγRIIB. In some aspects, the IgE-Fc region binds to the Fcε receptor with an association constant (Kₐ) from about 10⁸ M⁻¹ to about 10¹² M⁻¹. In some aspects, the IgE-Fc region further binds to a CD23 receptor. In some aspects, the IgE-Fc region binds to the CD23 receptor with an association constant (Kₐ) from about 10⁶ M⁻¹ to about 10¹⁰ M⁻¹. In some aspects, the IgE-Fc region is linked to the plurality of IgG Fc regions. In some aspects, a C-terminus of the IgE-Fc region is linked to an N-terminus of an IgG-Fc region of the plurality of IgG-Fc regions. In some aspects, an N-terminus of the IgE-Fc region is linked to a C-terminus of an IgG Fc region of the plurality of IgG-Fc regions. In some aspects, the IgE-Fc region is directly linked to the plurality of IgG Fc regions. In some aspects, the IgE-Fc region is covalently and directly linked to the plurality of IgG Fc regions. In some aspects, the IgE-Fc region is linked to the plurality of IgG Fc regions via a linker. In some aspects, the linker comprises one or more amino acid residues. In some aspects, the linker comprises the amino acid sequence set forth in SEQ ID NO: 13 or SEQ ID NO: 14. In some aspects, the linker comprises a hinge region of an immunoglobulin molecule. In some aspects, the hinge region is from an IgG-Fc region. Constructs comprising such hinge region linkers can have a reduced immunogenicity that can be due to (i) the orientation of the construct, e.g., an IgE-Fc region is C-terminally coupled to the IgG-Fc region, and (ii) the use of an IgG-Fc hinge region as a linker. In some aspects, the IgE-Fc region comprises a Cε2 domain, a Cε3 domain, a Cε4 domain, or a combination thereof. In some aspects, an IgG-Fc region of the plurality of IgG Fc regions comprises a Cγ2 domain, a Cγ3 domain, or a combination thereof. In some aspects, the IgE-Fc region comprises a Cε2 domain, a Cε3 domain, and a Cε4 domain, and an IgG-Fc region of the plurality of IgG Fc regions comprises a Cγ2 domain and a Cγ3 domain. In some aspects, the IgG-Fc region of the plurality of IgG Fc regions is from an Immunoglobulin G1 (IgG1), IgG2, IgG3, or IgG4 molecule. In some aspects, the IgG-Fc region of the plurality of IgG Fc regions is from an IgG1 molecule. In some aspects, the IgE-Fc region is a human IgE-Fc region. In some aspects, the human IgE-Fc region is a human wild-type IgE-Fc region. In some aspects, the human IgE-Fc region comprises at least one amino acid substitution, addition, and/or deletion, or a combination thereof. In some aspects, the at least one amino acid substitution comprises a T396F substitution. In some aspects, at least one IgG-Fc region of the plurality of IgG-Fc regions is a human IgG-Fc region. In some aspects, the at least one human IgG-Fc region of the plurality of IgG-Fc regions is a human wild-type IgG-Fc region. In some aspects, the at least one human IgG-Fc region comprises at least one amino acid substitution, addition, and/or deletion, or a combination thereof. In some aspects, the at least one amino acid substitution comprises a S267E, L328F, or an E333A substitution, or a combination thereof. In some aspects, the at least one amino acid substitution comprises a S267E and an L328F substitution. In some aspects, the plurality of IgG-Fc regions is capable of antibody-dependent cell-mediated cytotoxicity (ADCC)-mediated mast cell or basophil depletion. In some aspects, at least one IgG-Fc region of the plurality of IgG-Fc regions comprises a glutamic acid (E) to alanine (A) substitution relative to a human wild-type IgG-Fc region. In some aspects, the glutamic acid (E) to alanine (A) substitution is an E333A substitution. In some aspects, the plurality of IgG-Fc regions consists of two IgG-Fc regions. In some aspects, the plurality of IgG-Fc regions consists of three, four, or five IgG-Fc regions. In some aspects, the IgE-Fc region comprises an amino acid sequence having at least 80%, 85%, 90%, 95%, 97%, or 99% sequence identity or similarity to the amino acid sequence set forth in any one of SEQ ID NOs: 3-6, or 29. In some aspects, the IgE-Fc region comprises an amino acid sequence having at least 80%, 85%, 90%, 95%, 97%, or 99% sequence identity or similarity to the amino acid sequence set forth in SEQ ID NO: 29. In some aspects, the IgE-Fc region comprises the amino acid sequence set forth in SEQ ID NO: 29. In some aspects, an IgG-Fc region of the plurality of IgG-Fc regions comprises an amino acid sequence having at least 80%, 85%, 90%, 95%, 97%, or 99% sequence identity or similarity to the amino acid sequence set forth in any one of SEQ ID NOs: 9-10, or 26. In some aspects, the IgG-Fc region comprises an amino acid sequence having at least 80%, 85%, 90%, 95%, 97%, or 99% sequence identity or similarity to the amino acid sequence set forth in SEQ ID NO: 10. In some aspects, the IgG-Fc region comprises an amino acid sequence having at least 80%, 85%, 90%, 95%, 97%, or 99% sequence identity or similarity to the amino acid sequence set forth in SEQ ID NO: 26.

Further provided herein is a chimeric binding agent, wherein the chimeric binding agent comprises: (i) an IgE-Fc region, wherein the IgE-Fc region comprises a Cε2 domain, a Cε3 domain, a Cε4 domain, or a combination thereof; and (ii) at least one IgG-Fc region, wherein the at least one IgG-Fc region comprises a Cγ2 domain, a Cγ3 domain, or a combination thereof; wherein a C-terminus of the IgE-Fc region is linked to an N-terminus of the at least one IgG-Fc region. In some aspects, the IgE-Fc region is linked to the at least one IgG-Fc region. In some aspects, the IgE-Fc region is directly linked to the at least one IgG-Fc region. In some aspects, the IgE-Fc region is covalently and directly linked to the at least one IgG-Fc region. In some aspects, the IgE-Fc region is linked to the at least one IgG-Fc region via a linker. In some aspects, the linker comprises one or more amino acid residues. In some aspects, the linker comprises the amino acid sequence set forth in SEQ ID NO: 13 or SEQ ID NO: 14. In some aspects, the linker comprises a hinge region of an immunoglobulin molecule. In some aspects, the hinge region is from the at least one IgG-Fc region. In some aspects, the IgE-Fc region binds to an FcεR. In some aspects, the FcεR is an FcεRI. In some aspects, the at least one IgG-Fc region binds to an FcγR. In some aspects, the FcγR is an FcγRIIB In some aspects, the IgE-Fc region binds to the Fcε receptor with an association constant (Kₐ) from about 10⁸M⁻¹ to about 10¹² M⁻¹. In some aspects, the IgE-Fc region further binds to a CD23 receptor. In some aspects, the IgE-Fc region binds to the CD23 receptor with an association constant (Kₐ) from about 10⁶ M⁻¹ to about 10¹⁰ M⁻¹. In some aspects, the IgE-Fc region comprises a Cε2 domain, a Cε3 domain, and a Cε4 domain. In some aspects, the at least one IgG-Fc region comprises a Cγ2 domain and a Cγ3 domain. In some aspects, the at least one IgG-Fc region is from an Immunoglobulin G1 (IgG1), IgG2, IgG3, or IgG4 molecule. In some aspects, the at least one IgG-Fc region is from an IgG1 molecule. In some aspects, the IgE-Fc region is a human IgE-Fc region. In some aspects, the human IgE-Fc region is a human wild-type IgE-Fc region. In some aspects, the human IgE-Fc region comprises at least one amino acid substitution, addition, and/or deletion, or a combination thereof. In some aspects, the at least one amino acid substitution comprises a T396F substitution. In some aspects, the at least one IgG-Fc region is a human IgG-Fc region. In some aspects, the human IgG-Fc region is a human wild-type IgG-Fc region. In some aspects, the human IgG-Fc region comprises at least one amino acid substitution, addition, and/or deletion, or a combination thereof. In some aspects, the at least one amino acid substitution comprises a S267E, L328F, and/or E333A substitution, or a combination thereof. In some aspects, the human IgG-Fc region comprises a S267E and an L328F substitution. In some aspects, the at least one IgG-Fc region is capable of antibody-dependent cell-mediated cytotoxicity (ADCC)-mediated mast cell or basophil depletion. In some aspects, the at least one IgG-Fc region comprises a glutamic acid (E) to alanine (A) substitution relative to a human wild-type IgG-Fc region. In some aspects, the glutamic acid (E) to alanine (A) substitution is an E333A substitution. In some aspects, the at least one IgG-Fc region consists of one IgG-Fc region. In some aspects, the at least one IgG-Fc region consists of two IgG-Fc regions. In some aspects, the at least one IgG-Fc region consists of three, four, or five IgG-Fc regions. In some aspects, the IgE-Fc region comprises an amino acid sequence having at least 80%, 85%, 90%, 95%, 97%, or 99% sequence identity or similarity to the amino acid sequence set forth in any one of SEQ ID NOs: 3-6. In some aspects, the IgE-Fc region comprises an amino acid sequence having at least 80%, 85%, 90%, 95%, 97%, or 99% sequence identity or similarity to the amino acid sequence set forth in SEQ ID NO: 6. In some aspects, the IgE-Fc region comprises the amino acid sequence set forth in SEQ ID NO: 6. In some aspects, an IgG-Fc region of the plurality of IgG-Fc regions comprises an amino acid sequence having at least 80%, 85%, 90%, 95%, 97%, or 99% sequence identity or similarity to the amino acid sequence set forth in any one of SEQ ID NOs: 9-10, or 26. In some aspects, the IgG-Fc region comprises an amino acid sequence having at least 80%, 85%, 90%, 95%, 97%, or 99% sequence identity or similarity to the amino acid sequence set forth in SEQ ID NO: 10. In some aspects, the IgG-Fc region comprises an amino acid sequence having at least 80%, 85%, 90%, 95%, 97%, or 99% sequence identity or similarity to the amino acid sequence set forth in SEQ ID NO: 26. In some aspects, the chimeric binding agent comprises an amino acid sequence having at least 80%, 85%, 90%, 95%, 97%, or 99% sequence identity or similarity to the amino acid sequence set forth in SEQ ID NO: 21. In some aspects, the chimeric binding agent comprises an amino acid sequence having at least 80%, 85%, 90%, 95%, 97%, or 99% sequence identity or similarity to the amino acid sequence set forth in SEQ ID NO: 23. In some aspects, the chimeric binding agent comprises the amino acid sequence set forth in SEQ ID NO: 21. In some aspects, the chimeric binding agent comprises the amino acid sequence set forth in SEQ ID NO: 23.

In various aspects, the present disclosure provides a chimeric binding agent comprising an amino acid sequence having at least 80%, 85%, 90%, 95%, 97%, or 99% sequence identity or similarity to the amino acid sequence set forth in any one of SEQ ID NOs: 12, 25, or 28. In some aspects, the chimeric binding agent comprises an amino acid sequence having at least 80%, 85%, 90%, 95%, 97%, or 99% sequence identity to the amino acid sequence set forth in any one of SEQ ID NOs: 12, 25, or 28. In some aspects, the chimeric binding agent comprises an amino acid sequence having at least 80%, 85%, 90%, 95%, 97%, or 99% sequence identity to the amino acid sequence set forth in SEQ ID NO: 12. In some aspects, the chimeric binding agent comprises an amino acid sequence having at least 80%, 85%, 90%, 95%, 97%, or 99% sequence identity to the amino acid sequence set forth in SEQ ID NO: 25. In some aspects, the chimeric binding agent comprises an amino acid sequence having at least 80%, 85%, 90%, 95%, 97%, or 99% sequence identity to the amino acid sequence set forth in SEQ ID NO: 28. In some aspects, the chimeric binding agent comprises the amino acid sequence set forth in SEQ ID NO: 12. In some aspects, the chimeric binding agent comprises the amino acid sequence set forth in SEQ ID NO: 25. In some aspects, the chimeric binding agent comprises the amino acid sequence set forth in SEQ ID NO: 28.

In various aspects, the present disclosure provides a pharmaceutical composition comprising the chimeric binding agent of this disclosure and one or more pharmaceutically acceptable excipients. In some aspects, the chimeric binding agent is conjugated to a vehicle. In some aspects, the vehicle is a protein-based vehicle or a lipid-based vehicle. In some aspects, the pharmaceutical composition is a subcutaneous dosage form. In some aspects, the pharmaceutical composition is an intravenous dosage form. In some aspects, the pharmaceutical composition is an oral dosage form. In some aspects, an amount of the chimeric binding agent in the pharmaceutical composition is from about 0.01 mg/kg to about 10 mg/kg by weight of a subject. In some aspects, the subject is a rodent or a human.

Further provided herein is a method of treating or preventing inflammation in a subject in need thereof, the method comprising administering to the subject a pharmaceutical composition of this disclosure comprising a chimeric binding agent, wherein the pharmaceutical composition is present in an effective amount for treating or preventing the inflammation in the subject. In some aspects, the method further comprises administering a bacterial consortium to the subject. In some aspects, the bacterial consortium comprises Lactobacillus sp., Faecalibacterium sp., or Akkermansia sp., or a combination thereof. In some aspects, the inflammation is an allergic inflammation. In some aspects, the allergic inflammation is allergic asthma or allergic airway inflammation. In some aspects, the inflammation is an IgE-mediated inflammatory disease. In some aspects, the IgE-mediated inflammatory disease is Hyper-IgE-Syndrome. In some aspects, the subject is a mammal. In some aspects, the subject is a rodent or a human.

Also provided herein is a method of depleting FceRI+ cells in a cell population, the method comprising contacting the cell population with a composition comprising a chimeric binding agent herein, wherein the composition depletes the FceRI+ cells in the cell population via antibody-dependent cell-mediated cytotoxicity (ADCC), and wherein depleting the FceRI+ cells in the cell population comprises depleting at least 30% of FceRI+ cells in the cell population after contacting the cell population with the composition for 12 hours. In some aspects, the composition comprises a chimeric binding agent that comprises the amino acid sequence set forth in SEQ ID NO: 23 or 28. In some aspects, the cell population is from a mammal. In some aspects, the cell population is from a rodent or a human. In some aspects, the composition depletes at least 50% of FceRI+ cells in the cell population after 6 hours.

The present disclosure provides a method for treating a subject having or suspected of having a disorder, comprising administering to the subject a therapeutically-effective amount of a chimeric binding agent comprising i) a first region that has binding specificity with at least one functional domain of an Immunoglobulin E (IgE) molecule; and ii) a second region that has binding specificity with at least one functional domain of an Immunoglobulin G (IgG) molecule, which binding agent binds an Fc epsilon (Fcε) receptor and an Fc gamma (Fcγ) receptor in the subject. The binding of the Fcε receptor may suppress or reduce inflammation in the subject. The Fcε receptor may be an FcεRI receptor. The binding to the Fcε receptor may inhibit cell degranulation. The chimeric binding agent may further comprise a linker region connecting the at least one functional domain of the IgE molecule to the at least one functional domain of the IgG molecule. The first region may have at least 90%, 95%, 97%, or 99% sequence identity to at least one functional domain of the IgE. The first region may have at least 90%, 95%, 97%, or 99% sequence identity to at least one functional domain of the IgG. The at least one functional domain of the IgG molecule may comprise a Cγ3 domain from the IgG molecule. The Cγ2 domain from the IgG molecule may comprise at least one amino acid variation compared to a wild-type sequence of the IgG molecule. The at least one amino acid variation may comprise a S267E variation. The at least one amino acid variation may comprise a L328F variation. The at least one amino acid variation may comprise a S267E variation and a L328F variation. The second region may comprise an amino acid sequence having at least 80%, 85%, 90%, 95%, 97%, or 99% sequence identity to any one of SEQ ID NO: 8-SEQ ID NO: 10. The at least one functional domain of the IgE molecule may comprise a Cε3 domain from the IgE molecule. The Cε3 domain from the IgE molecule may comprise at least one amino acid variation compared to a wild-type sequence of the IgE molecule. The at least one amino acid variation may comprise a T396F variation. The at least one functional domain of the IgE molecule may comprise a Cε4 domain from the IgE molecule. The Cε4 domain from the IgE molecule may comprise at least one amino acid variation as compared to a wild-type sequence. The at least one functional domain of the IgE molecule may comprise two or more of a Ca domain from the IgE molecule, a Cε3 domain from the IgE molecule, or a Cε4 domain from the IgE molecule. The first region may comprise an amino acid sequence having at least 80%, 85%, 90%, 95%, 97%, or 99% sequence identity to any one of SEQ ID NO: 3-SEQ ID NO: 6. Each of the two or more functional domains may be linked via a flexible linker. Each of the flexible linker may comprise a repeat of GGGGS (SEQ ID NO: 13) sequences. Each of the repeat of GGGGS (SEQ ID NO: 13) sequences may be a triple repeat (G4S)3 (SEQ ID NO: 14). The Cε3 domain from the IgE molecule or the Cε4 domain from the IgE molecule may bind to the Fcε receptor. The Cε3 domain from the IgE molecule or the Cε4 domain from the IgE molecule may bind to the Fcε receptor with an association constant (Ka) from about 10⁸M⁻¹ to about 10¹² M⁻¹. The chimeric binding agent may further bind to an Fc gamma (Fcγ) receptor. The Fcγ receptor may be an FcγRIIB receptor. The binding to the FcγRIIB receptor may trigger ITIM phosphorylation within a cell of the subject. The chimeric binding agent may further bind to a CD23 receptor. The chimeric binding agent may further bind to a CD23 receptor with an association constant from about 10⁶ M⁻¹ to about 10¹⁰ M⁻¹. The IgG molecule may be an Immunoglobulin G1 (IgG1) molecule. The IgG molecule may be an Immunoglobulin G2 (IgG2) molecule. The IgG molecule may be an Immunoglobulin G3 (IgG3) molecule. The IgG molecule may be an Immunoglobulin G4 (IgG4) molecule. The disorder may be an immune disorder. The immune disorder may be allergic inflammation. The immune disorder may exhibit an inflammatory response in the subject. The therapeutically-effective amount may be from about 0.01 mg/kg to about 10 mg/kg by weight of the subject. The subject may be a human. The chimeric binding agent may be administered in a composition, and wherein the composition comprises a pharmaceutically-acceptable carrier. The chimeric binding agent may be conjugated to a vehicle. The vehicle may be a lipid-based vehicle. The chimeric binding agent may be administered by subcutaneous injection. The binding agent may simultaneously bind the Fc epsilon (Fcε) receptor and the Fc gamma (Fcγ) receptor in the subject. The chimeric binding agent may comprise an amino acid sequence set forth in SEQ ID NO: 12.

The present disclosure provides a method of treating a disorder in a subject comprising administering to the subject a therapeutically effective amount of a chimeric binding agent that substantially simultaneously binds to an Fc epsilon (Fcε) receptor and to an Fc gamma (Fcγ) receptor, thereby inhibiting the Fcε receptor while activating the Fcγ receptor. The binding to the Fcε receptor may trigger a crosslinking of the Fcε receptor with the Fcγ receptor. The binding to the Fcε receptor may inhibit cell degranulation. The Fcε receptor may be an FcεRI receptor. The FcεRI receptor may be from a mast cell. The FcεRI receptor may be from a basophil cell. The Fcγ receptor may be an FcγRIIB receptor. The binding to the FcγRIIB receptor may trigger ITIM phosphorylation within a cell of the subject. ITIM phosphorylation may activate a signaling pathway that results in suppression of an effector cell. The chimeric binding agent may further comprise a linker connecting at least one functional domain that binds to the Fcε receptor and at least one functional domain that binds to the Fcγ receptor. The at least one functional domain that binds to the Fcγ receptor may comprise a Cγ2 domain from an IgG molecule. The Cγ2 domain from the IgG molecule may further comprise at least one amino acid variation as compared to a wild-type sequence. The at least one amino acid variation may comprise a S267E variation. The at least one amino acid variation may comprise a L328F variation. The at least one amino acid variation may comprise a S267E variation and a L328F variation. The chimeric binding agent may comprise an amino acid sequence having at least 80%, 85%, 90%, 95%, 97%, or 99% sequence identity to any one of SEQ ID NO: 8-SEQ ID NO: 10. The at least one functional domain that binds to the Fcε receptor may comprise a Cε3 domain from an IgE molecule. The Cε3 domain from the IgE molecule further may comprise at least one amino acid variation as compared to a wild-type sequence. The at least one amino acid variation may comprise a T396F variation. The at least one functional domain that binds to the Fcε receptor may comprise a Cε3 domain from an IgE molecule or a Cε4 domain from an IgE molecule. The at least one functional domain that binds to the Fcε receptor may comprise two or more of a Ca domain from an IgE molecule, a Cε3 domain from an IgE molecule, or a Cε4 domain from an IgE molecule. The chimeric binding agent may comprise an amino acid sequence having at least 80%, 85%, 90%, 95%, 97%, or 99% sequence identity to any one of SEQ ID NO: 3-SEQ ID NO: 6. Each of the two or more functional domains may be linked via a flexible linker. Each of the flexible linker may comprise a repeat of GGGGS (SEQ ID NO: 13) sequences. Each of the repeat of the GGGGS (SEQ ID NO: 13) sequences may be a triple repeat (G4S)3 (SEQ ID NO: 14). The at least one functional domain that binds to the Fcγ receptor may be a functional domain from an Immunoglobulin G (IgG) molecule. The IgG molecule may be an Immunoglobulin G1 (IgG1) molecule, an Immunoglobulin G2 (IgG2) molecule, an Immunoglobulin G3 (IgG3) molecule, or an Immunoglobulin G4 (IgG4) molecule. The disorder may be an immune disorder. The disorder may be asthma or allergic inflammation. The subject may be a human. The chimeric binding agent may comprise an amino acid sequence set forth in SEQ ID NO: 12.

The present disclosure provides a chimeric binding agent for binding to an Fc epsilon (Fcε) receptor and to an Fc gamma (Fcγ) receptor that may comprise: i) a first region that has binding specificity with at least one functional domain of an Immunoglobulin E (IgE) molecule; and ii) a second region that has binding specificity with at least one functional domain of an Immunoglobulin G (IgG) molecule; wherein the chimeric binding agent may be formulated for administration to a subject, and wherein upon administration to the subject, the binding agent binds an Fc epsilon (Fcε) receptor and an Fc gamma (Fcγ) receptor in the subject. The first region may have at least 80%, 85%, 90%, 95%, 97%, or 99% sequence identity to the at least one functional domain of the IgE molecule. The first region that has at least 80%, 85%, 90%, 95%, 97%, or 99% sequence identity to the at least one functional domain of the IgE molecule may be from a human. The first region that has at least 80%%, 85%, 90%, 95%, 97%, or 99% sequence identity to the at least one functional domain of the IgE molecule may be from a rodent. In some instances, the at least one functional domain of the IgE molecule may be humanized. Such humanized functional domain may be based on a murine amino acid sequence. The second region may have at least 80%, 85%, 90%, 95%, 97%, or 99% sequence identity to the at least one functional domain of the IgG molecule. The second region that has at least 80%, 85%, 90%, 95%, 97%, or 99% sequence identity to the at least one functional domain of the IgG molecule may be from a human. The second region that has at least 80%, 85%, 90%, 95%, 97%, or 99% sequence identity to the at least one functional domain of the IgG molecule may be from a rodent. In some instances, the at least one functional domain of the IgG molecule may be humanized. Such humanized functional domain may be based on a murine amino acid sequence. The Fcγ receptor may be an FcγRIIB receptor. The Fcε receptor may be an FcεRI receptor. The IgG molecule may be an Immunoglobulin G1 (IgG1) molecule. The IgG molecule may be an Immunoglobulin G2 (IgG2) molecule. The IgG molecule may be an Immunoglobulin G3 (IgG3) molecule. The IgG molecule may be an Immunoglobulin G4 (IgG4) molecule. The chimeric binding agent may further comprise a linker connecting the first region that has at least 80%, 85%, 90%, 95%, 97%, or 99% sequence identity to at least one functional domain of an IgE molecule to the second region that has at least 80%, 85%, 90%, 95%, 97%, or 99% sequence identity to at least one functional domain of an IgG molecule. The linker may be a flexible linker. The flexible linker may comprise a repeat of GGGGS (SEQ ID NO: 13) sequences. Each of the repeat of the GGGGS (SEQ ID NO: 13) sequences may be a triple repeat (G4S)3 (SEQ ID NO: 14). The at least one functional domain of the IgG molecule may comprise a Cγ2 domain from the IgG molecule. The Cγ2 domain from the IgG molecule may comprise at least one amino acid variation as compared to a wild-type sequence. The at least one amino acid variation may comprise a S267E variation. The at least one amino acid variation may comprise a L328F variation. The at least one amino acid variation may comprise a S267E variation and a L328F variation. The at least one functional domain of the IgE molecule may comprise a Cε3 domain from the IgE molecule. The Cε3 domain from the IgE molecule may comprise at least one amino acid variation as compared to a wild-type sequence. The at least one amino acid variation may comprise a T396F variation. The at least one functional domain of the IgE molecule may comprise a Cε2 domain from the IgE molecule. The at least one functional domain of the IgE molecule may comprise a Cε4 domain from the IgE molecule. The at least one functional domain of the IgE molecule may comprise two or more of a Cε2 domain from the IgE molecule, a Cε3 domain from the IgE molecule, or a Cε4 domain from the IgE molecule. The first region may comprise an amino acid sequence having at least 80%, 85%, 90%, 95%, 97%, or 99% sequence identity to any one of SEQ ID NO: 3-SEQ ID NO: 6. The second region may comprise an amino acid sequence having at least 80%, 85%, 90%, 95%, 97%, or 99% sequence identity to any one of SEQ ID NO: 8-SEQ ID NO: 10. The chimeric binding agent may comprise an amino acid sequence set forth in SEQ ID NO: 12.

The present disclosure provides a method of treating a subject having or suspected of having a disorder comprising administering to the subject a therapeutically-effective amount of a chimeric binding agent, wherein the chimeric binding agent binds to an Fc epsilon (Fcε) receptor and an Fc gamma (Fcγ) receptor in the subject.

The present disclosure also provides a pharmaceutical composition comprising a chimeric binding agent as described herein, wherein the chimeric binding agent is configured to bind to an Fc epsilon (Fcε) receptor and an Fc gamma (Fcγ) receptor in a subject.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIGS. 1A-1G illustrate different immunoglobulin (Ig)-Fc binding agent formats of the present disclosure. FIG. 1A schematically illustrates an immunoglobulin G (IgG)-Fc protein comprising, e.g., the functional domains Cγ2 and Cγ3 and a hinge region. FIG. 1B schematically illustrates a polypeptide structure in which an immunoglobulin E (IgE)-Fc protein (e.g., Fcε) comprises an IgE region comprising functional IgE domains (e.g., Cε2, Cε3, and Cε4) and a hinge region (e.g., those comprising amino acid sequences set forth in any one of SEQ ID NO: 3-SEQ ID NO: 6). FIG. 1C schematically illustrates a bivalent IgG-Fc-IgE-Fc fusion protein (e.g., chimeric binding agent) comprising a first region of an IgE molecule comprising IgE functional domains (e.g., Cε2, Cε3, and Cε4) and a second region comprising IgG functional domains (e.g., a hinge region, Cγ2 and Cγ3). Such fusion polypeptides can comprise an amino acid sequence set forth in any one of SEQ ID NO: 3-SEQ ID NO: 6), wherein both Fc portions may be fused via a linker (e.g., a flexible linker) that may comprise a triple repeat of GGGGS, referred to as (G₄S)₃ (SEQ ID NO: 14). FIG. 1D schematically illustrates a bivalent IgE-IgG-Fc binding agent in which, from the N-terminus to the C-terminus, an IgE-Fc portion is coupled to an IgG-Fc portion via the IgG-Fc hinge region. FIG. 1E schematically illustrates the structure of a bivalent IgG-IgE-Fc binding agent that comprises an IgG-Fc portion comprising a S267E and a L328F (i.e., “SELF”; as shown in the gray domains) substitution. Such substitutions can provide increased FcγRIIB binding affinities (e.g., a 166-fold increase) of the IgG-Fc portion compared to wild-type IgG-Fc domains. FIG. 1F schematically illustrates the structure of a bivalent IgE-IgG-Fc binding agent in which, from the N-terminus to the C-terminus, an IgE-Fc portion is coupled to an IgG-Fc portion via the IgG-Fc hinge region, and that comprises an IgG-Fc portion with a S267E and a L328F (i.e., “SELF”) substitution. FIG. 1G schematically illustrates the structure of a trivalent (IgG-Fc)₂-IgE-Fc binding agent (the order of the Ig-Fc portions are described form N- to C-terminus) in which the IgG-Fc portions can be coupled to the IgE-Fc portion using various linker strategies, including (G₄S)₃ linkers.

FIGS. 2A-B schematically illustrate situations in which chimeric Fc fusion proteins of the present disclosure may prevent IgE-mediated degranulation through co-ligation of activating and inhibitory Fc Receptors. FIG. 2A illustrates that antigen-specific IgE molecules bind to FcεRI receptors expressed on cells such as immune cells (e.g., mast cells and basophils). Upon re-exposure to the antigen, the receptor-bound IgE binds the antigen, leading to cross-linking of the Fc epsilon receptors. Phosphorylation of immunoreceptor tyrosine-based activation motif (ITAM) of the FcεRI receptor's γ-chain triggers a signaling cascade that ultimately results in cell degranulation. FIG. 2B illustrates that the chimeric Fc fusion proteins of the present disclosure bind with their IgE Fc portion to FcεRIs, thereby blocking these receptors from their interaction with antigen-specific and/or antigen-bound IgE molecules. Additionally, the IgG Fc portion of the chimeric Fc fusion protein (e.g., chimeric binding agent) selectively binds to the inhibitory receptor FcγRIIB Engagement and co-ligation of FcγRIIB triggers immunoreceptor tyrosine-based inhibition motif (ITIM) phosphorylation which induces a signaling pathway resulting in inactivation and thus suppression of the effector cell.

FIGS. 3A-B schematically illustrate a design and structure of expression vectors that may be used for the expression of IgE-Fc and chimeric IgG-IgE-Fc fusion proteins as described herein.

FIG. 3A illustrates the design and structure of expression vectors for murine IgE-Fc proteins.
FIG. 3B illustrates the design and structure of expression vectors for murine chimeric IgG-IgE-Fc fusion proteins.

FIGS. 4A-F illustrate the development of cell lines that stably express the respective IgE-Fc (e.g., those comprising an amino acid sequence set forth in SEQ ID NO: 5) and chimeric IgG-Fc-IgE-Fc (e.g., those comprising an amino acid sequence set forth in SEQ ID NO: 12) chimeric fusion proteins for high yield production of these proteins. FIG. 4A illustrates HEK293T cells that were stably transfected with plasmid vectors coding for either the single region IgE-Fc (e.g., those comprising an amino acid sequence set forth in SEQ ID NO: 5) or IgG-Fc-IgE-Fc (e.g., those comprising an amino acid sequence set forth in SEQ ID NO: 12) chimeric fusion proteins. After 12 days of cell selection, in both cell lines (those producing either IgE-Fc or the IgG-Fc-IgE-Fc chimeric protein, respectively) 99.4% of cells in the respective culture were positive for the expression cassette. FIG. 4B illustrates the screening of sub-cloned cell line candidates for protein production levels in the cell culture supernatants by ELISA. The cell sub-clones producing the highest yield of IgE-Fc were again selected, expanded and used for protein production. FIG. 4C illustrates the screening of sub-cloned cell line candidates for protein production levels in the cell culture supernatants by ELISA. The cell sub-clones producing the highest yield of IgG-Fc-IgE-Fc were again selected, expanded and used for protein production. FIG. 4D illustrates a Coomassie blue staining confirming the calculated size and integrity of the purified proteins under non-reducing conditions for IgE-Fc (˜70 kDa) and the IgG-Fc-IgE-Fc chimeric fusion (˜135 kDa). FIG. 4E shows that selected cells were sub-cloned and the clones with the highest mean fluorescence intensity of GFP (e.g., G4, G6, and G8 clones) were selected and further expanded for production of chimeric binding agents comprising a human (“h”) IgG-Fc domain and a murine (“m”) IgE-Fc domain. These candidates were then screened for protein production levels in the cell culture supernatants by ELISA. The clones producing the highest yield of binding agent (e.g., G6 or G8 clones) were again selected, expanded and used for drug production. FIG. 4F shows cell line development and yield (in ng/mL) after 4 days of production of chimeric binding agents comprising a human IgG-Fc portion comprising a S267E and a L328F (i.e., “SELF”) substitution.

FIGS. 5A-B illustrate the binding affinity of the expressed fusion proteins to Fc receptors. FIG. 5A illustrates that IgG-Fc-IgE-Fc fusion proteins (e.g., those comprising an amino acid sequence set forth in SEQ ID NO: 12) were able to bind to receptors on cells of the rat basophil, on cells of the cell line RBL-2H3, and to those on the human peripheral blood mononuclear cells (PBMCs) even at very low protein concentrations in which the IgG-Fc-IgE-Fc fusion protein was present in the cell culture supernatants compared to the purified and highly concentrated IgE-Fc proteins. FIG. 5B illustrates the binding affinity of purified IgE-Fc protein to mouse Fc epsilon receptor 1 a-chain (mFCER1A) as analyzed by ELISA. Plates were coated with increasing concentrations of mFCER1A protein and probed with IgE-Fc protein. Binding affinity of the IgE-Fc protein to the Fc epsilon receptor was compared to commercially available IgE antibodies (clone C48-2 and clone C38-2) which served as positive controls. PNGaseF-treated deglycosylated IgE clone C48-2 served as negative control.

FIGS. 6A-C illustrate bar graphs confirming that blocking of Fc receptors with a chimeric binding agent as disclosed herein suppresses IgE-mediated allergic inflammation. For these studies, RBL-2H3 cells were sensitized with 1 μg/ml 2,4,6-trinitrophenyl (TNP)-specific IgE and then challenged with 2 μg/ml TNP-Keyhole Limpet Hemocyanin (KLH) antigen (e.g., 2,4,6-trinitrophenyl hapten conjugated to KHL protein via lysine conjugation). FIG. 6A illustrates that pre-treatment (grey bars, black bars show control without IgE block) of unspecific IgE efficiently inhibited IgE-mediated degranulation by measuring beta-hexosaminidase release into the extracellular medium after challenge with the TNP-KLH antigen (abbreviated here as “Ag”). Prior to sensitization with IgE, cells were either treated with PBS (black bars) or with 1 μg/ml non-specific IgE to saturate and block Fc epsilon receptors (grey bars). FIG. 6B illustrates complete inhibition of degranulation using equimolar concentrations of purified IgE-Fc protein and hapten. FIG. 6B further illustrates that inhibition of degranulation occurred in a dose-dependent manner, indicating that full saturation of Fc receptors may block antigen-specific antibodies from binding and activating the cells. FIG. 6C illustrates that induction of degranulation was measured efficiently when antibodies were fully glycosylated but and that it was significantly reduced when the antibodies were aglycosylated. FIG. 6C further illustrates the same effect for pre-treated cells, which were protected from IgE-mediated degranulation when treated with fully glycosylated IgE-Fc but lost protection when aglycosylated IgE-Fc was used.

FIGS. 7A-D illustrate in vitro degranulation assays to demonstrate reduction of allergic reactions using in vitro allergy models. RBL-2H3 cells were sensitized with 1 μg/ml TNP-specific IgE and challenged with 2 μg/ml antigen (TNP-KLH). The proteins IgE-Fc (SEQ ID NO: 5) and IgG-Fc-IgE-Fc (SEQ ID NO: 12, also referred to herein as “compound A”) as well as antigen-specific IgE (anti-TNP-KLH, purchased from BD Biosciences) were used in equimolar concentrations. FIG. 7A illustrates beta-hexanosaminidase release of RBL-2H3 cells after exposure to the fusion proteins IgE-Fc and IgG-Fc-IgE-Fc in a preventative treatment model. Following antigen challenge, β-hexosaminidase release in the supernatant was quantified and used as a way of degranulation. Both fusion proteins IgE-Fc (SEQ ID NO: 5) and IgG-Fc-IgE-Fc (SEQ ID NO: 12, compound A) reduced β-hexosaminidase release comparable to full length IgE antibodies. FIG. 7B shows that cells received the Ns-IgE and the fusion proteins IgE-Fc and IgG-Fc-IgE-Fc simultaneously with antigen-specific antibodies. Thus, the Ns-IgE and the fusion proteins IgE-Fc (SEQ ID NO: 5) and IgG-Fc-IgE-Fc (SEQ ID NO: 12, compound A) were competing with antigen-specific antibodies for the FcR binding sites. FIG. 7C shows the percent beta-hexanosaminidase release in a preventative (bar nr. 3-5) and a therapeutic (bar nr. 6-8) setting in which the fusion proteins IgE-Fc (SEQ ID NO: 5) and IgG-Fc-IgE-Fc (SEQ ID NO: 12, compound A) are administered after antigen challenge. FIG. 7D shows the percent degranulation of RBL-2H3 cells in a preventative (bar nr. 3-5) and a therapeutic (bar nr. 6-8) setting in which the fusion proteins IgE-Fc (SEQ ID NO: 5) and IgG-Fc-IgE-Fc (SEQ ID NO: 12, compound A) are administered after antigen challenge.

FIG. 8 shows the mean fluorescence intensity (MFI) using the cytoplasmic dye carboxyfluorescein diacetate succinimidyl ester (CFSE) of Rat Basophilic Leukemia (RBL) cells untreated or treated with the chimeric binding agents which amino acid sequence is set forth in SEQ ID NO: 12 over the course of 3 days. The binding agent was used in concentrations of 10 protein.

FIG. 9A shows fold change of FcεR1a expression in RBL-2H3 cells under various conditions: (i) without (w/o) treatment; (ii) treated; (iii) treated with the chimeric binding agent having the sequence set forth in SEQ ID NO: 12; and (iv) treated with IgE and the chimeric binding agent having the sequence set forth in SEQ ID NO: 12. FIG. 9B shows the expression of FcεR1a as mean fluorescence intensity (MFI) in RBL-2H3 cells under various conditions: (i) without (w/o) treatment; (ii) treated; (iii) treated with the chimeric binding agent having the sequence set forth in SEQ ID NO: 12; and (iv) treated with IgE and the chimeric binding agent having the sequence set forth in SEQ ID NO: 12.

FIG. 10A shows percent cell viability of RBL-2H3 cells under various conditions: (i) without (w/o) treatment (RBL cells alone); (ii) RBL cells treated with the chimeric binding agent having the sequence set forth in SEQ ID NO: 28 (also referred to herein as “compound B”) and comprising an E333A substitution in a wild-type IgG-Fc region; (iii) RBL cells and PBMCs alone; and (iv) RBL cells and PBMCs treated with the chimeric binding agent having the sequence set forth in SEQ ID NO: 28 (compound B). FIG. 10B shows a microscopy image of untreated RBL-2H3 cells after 6 hours. FIG. 10C shows a microscopy image of RBL-2H3 cells after 6 hours that were treated with the chimeric binding agent having the sequence set forth in SEQ ID NO: 28 (compound B). FIG. 10D shows a microscopy image of RBL-2H3 cells in the presence of PBMCs after 6 hours. FIG. 10E shows a microscopy image of RBL-2H3 cells after 6 hours that were treated with both PBMCs and the chimeric binding agent having the sequence set forth in SEQ ID NO: 28 (compound B).

FIG. 11 provides an overview of an in vivo study using a mouse model of allergic airway inflammation.

FIGS. 12A-12E show serum levels various T cells, B cells, as well as serum IgE levels of C57BL/6J mice from three different cohorts: (i) mice that received no treatment and were immunized with PBS (CTRL cohort); (ii) mice that were immunized with aluminum hydroxide (alum)-emulsified ovalbumin (OVA) and received vehicle PBS via intraperitoneal (i.p.) injection (PBS cohort); or (iii) mice that were OVA immunized and received the chimeric binding agent consisting of the amino acid sequence set forth in SEQ ID NO: 12 (5 mg/kg) via i.p. injection (compound A cohort). FIG. 12A shows the percentage of serum T follicular helper cells that stained positive for the markers CD3, CD4, CXCR5, and PD-1 in the three different cohorts of C57BL/6J mice. FIG. 12B shows the percentage of serum germinal center B cells that stained positive for the markers B220, Fas, GL7 and negative for IgM and IgD in the three different cohorts of C57BL/6J mice. FIG. 12C shows the percentage of serum T helper cells that stained positive for the markers CD3, CD4, IL-4, and GATA-3 in the three different cohorts of C57BL/6J mice. FIG. 12D shows the total serum IgE concentration in the three different cohorts of C57BL/6J mice. FIG. 12E shows OVA-specific IgE serum levels given as relative optical density (OD, at 450 nm) in the three different cohorts of C57BL/6J mice.

FIGS. 13A-13B show the chimeric binding agent consisting of the amino acid sequence set forth in SEQ ID NO: 12 is detectable on FcεRI-positive mast cells obtained from the three different cohorts: (i) mice that received no treatment and were immunized with PBS (CTRL cohort); (ii) mice that were immunized with aluminum hydroxide (alum)-emulsified ovalbumin (OVA) and received vehicle PBS via intraperitoneal (i.p.) injection (PBS cohort); or (iii) mice that were OVA immunized and received the chimeric binding agent consisting of the amino acid sequence set forth in SEQ ID NO: 12 (5 mg/kg) via i.p. injection (compound A cohort). FIG. 13A shows the percentage of mast cells that stained positive for the markers c-kit, FcεRI, and CD49b in serum of C57BL/6J mice from three different cohorts. FIG. 13B shows detection of α-His-SEQ ID NO: 12 binding agent shown as mean fluorescence intensity on lung mast cells of C57BL/6J mice from three different cohorts. Flow cytometry experiments were CD45⁺ c-kit⁺ FcεRI⁺ CD49b⁺ gated.

FIGS. 14A-14B show the chimeric binding agent consisting of the amino acid sequence set forth in SEQ ID NO: 12 is detectable on FcεRI-positive basophils obtained from the three different cohorts: (i) mice that received no treatment and were immunized with PBS (CTRL cohort); (ii) mice that were immunized with aluminum hydroxide (alum)-emulsified ovalbumin (OVA) and received vehicle PBS via intraperitoneal (i.p.) injection (PBS cohort); or (iii) mice that were OVA immunized and received the chimeric binding agent consisting of the amino acid sequence set forth in SEQ ID NO: 12 (5 mg/kg) via i.p. injection (compound A cohort). FIG. 14A shows the percentage of mast cells that stained positive for the markers, FcεRI, CD49b, and CD123 in serum of C57BL/6J mice from three different cohorts. FIG. 14B shows detection of α-His-SEQ ID NO: 12 binding agent shown as mean fluorescence intensity on lung basophils of C57BL/6J mice from three different cohorts. Flow cytometry experiments were CD45⁺ FcεRI⁺ CD49b⁺ CD123⁺ gated.

FIGS. 15A-15B show that the chimeric binding agent consisting of the amino acid sequence set forth in SEQ ID NO: 12 prevented immediate responses to allergen challenge in vivo as shown in the three different C57BL/6J mouse cohorts: (i) mice that received no treatment and were immunized with PBS (CTRL cohort); (ii) mice that were immunized with aluminum hydroxide (alum)-emulsified ovalbumin (OVA) and received vehicle PBS via intraperitoneal (i.p.) injection (PBS cohort); or (iii) mice that were OVA immunized and received the chimeric binding agent consisting of the amino acid sequence set forth in SEQ ID NO: 12 (5 mg/kg) via i.p. injection (compound A cohort). FIG. 15A shows IL-4 concentrations (in pg/mL) in lung tissue from the three different cohorts. FIG. 15B shows serum histamine concentrations (in pg/mL) in the three different cohorts.

FIGS. 16A-16B show that the chimeric binding agent consisting of the amino acid sequence set forth in SEQ ID NO: 12 prevented recruitment of eosinophils and neutrophils to the lung as shown in the three different C57BL/6J mouse cohorts: (i) mice that received no treatment and were immunized with PBS (CTRL cohort); (ii) mice that were immunized with aluminum hydroxide (alum)-emulsified ovalbumin (OVA) and received vehicle PBS via intraperitoneal (i.p.) injection (PBS cohort); or (iii) mice that were OVA immunized and received the chimeric binding agent consisting of the amino acid sequence set forth in SEQ ID NO: 12 (5 mg/kg) via i.p. injection (compound A cohort). FIG. 16A shows the percentage of lung eosinophils that stained positive for the markers CD11b and SiglecF and negative for Cd11c in serum of C57BL/6J mice from three different cohorts. FIG. 16B shows the percentage of lung neutrophils that stained positive for the markers CD11b and Gr-1 and negative for Cd11c in serum of C57BL/6J mice from three different cohorts.

### DETAILED DESCRIPTION

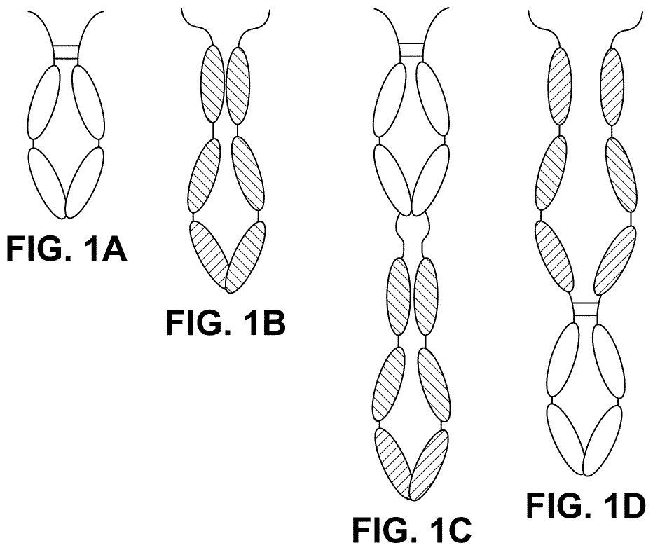

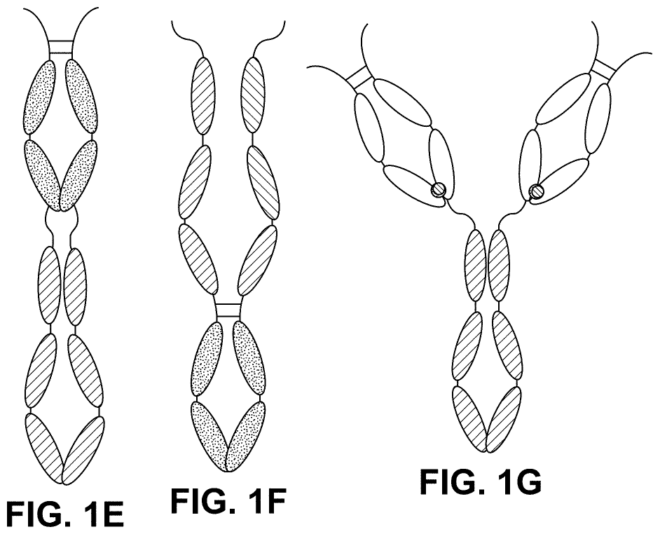

Provided herein are compositions comprising a multivalent chimeric binding agent comprising a plurality of immunoglobulin (Ig) fragment crystallizable (Fe) regions. Each Ig-Fc region of the plurality of Ig-Fc regions can be from an IgA, IgG, or IgE molecule. The multivalent chimeric binding agents provided herein are capable of binding one or more different Fc receptors (FcRs). In various instances, a multivalent chimeric binding agent can comprise or consist of at least one IgG-Fc region and at least one IgE-Fc region. Such binding agents can be capable of binding an Fcγ-receptor and an Fcε-receptor. The two or more Fc regions of a binding agent can be coupled to one another directly or indirectly and covalently or non-covalently. In some instances, provided herein is a bivalent binding agent comprising, from the N- to the C-terminus, an IgG-Fc region indirectly and covalently coupled to an IgE-Fc region via linker, e.g., as shown in FIG. 1C. In other instances, provided herein is a bivalent binding agent comprising, from the N- to the C-terminus, an IgE-Fc region directly and covalently coupled to an IgG-Fc region via the N-terminal hinge region of the IgG-Fc portion, e.g., as shown in FIG. 1D. In yet other instances, provided herein are trivalent binding agents comprising or consisting of, from the N- to the C-terminus, at least two IgG-Fc regions indirectly coupled to an IgE-Fc region via linkers, e.g., as shown in FIG. 1G.

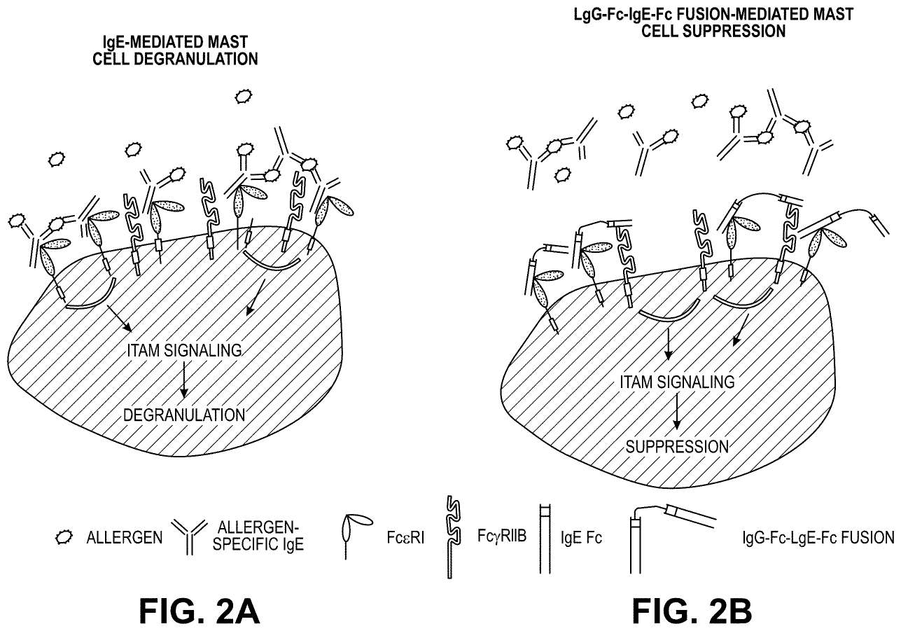

The multivalent chimeric binding agents provided herein can have one or more advantages over convention therapeutic approaches to treat inflammation, autoimmune diseases, etc., such advantages include (i) an increased serum half-life mediated by IgG-Fc enabling FcRn engagement; (ii) prolonged binding to targets cells (e.g., mast cells and basophils) through simultaneous interaction with two different receptors, such receptors can include Fcε RI and FcγRIIB receptors (see e.g., FIGS. 2A-B); (iii) low or no measurable immunogenicity, e.g., when using fully human Fc regions; (iv) targeting of effector cells directly through IgE-FcεR specific interactions; and (v) interactions with targeted FcRs with increased receptor selectivity and affinity through engineered Fc fragments, including “SELF” mutations in IgG regions of a chimeric binding agent. Moreover, and without being bound to any theory, it is assumed that the chimeric binding agents of the present disclosure provide superior anti-inflammatory properties compared to conventional treatments. Such superior properties may be due to inhibition of Fcε RI upregulation and expression in effector cells (e.g., mast cells and basophils), effectively preventing endogenous IgE from binding to FcεRs, and inactivation of effector cells by co-ligating Fcε RIs and FcγRIIBs on effector cells simultaneously.

In some instances, a composition herein comprising one or more multivalent chimeric binding agents can further comprise one or more bacterial species. Such one or more bacterial species can include one or more species of Lactobacillus sp., Faecalibacterium sp., and/or Akkermansia sp.

Further provided herein are methods for treating and reducing the incidence of various diseases in a subject in need thereof by administering a composition comprising a chimeric binding agent as described herein. Such diseases can include inflammatory diseases and autoimmune diseases. In various cases, provided herein are methods of treating or reducing the incidence of allergy such as allergic airway inflammation or allergic asthma in a subject.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The abbreviations for the natural L-enantiomeric amino acids as used herein are conventional and are as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); and valine (V, Val). Typically, Xaa may indicate any amino acid. X may be asparagine (N), glutamine (Q), histidine (H), lysine (K), or arginine (R).

The present disclosure may contemplate D-amino acid residues of any standard or non-standard amino acid or analogue thereof. When an amino acid sequence is represented as a series of three-letter or one-letter amino acid abbreviations, the left-hand direction is the amino terminal direction and the right-hand direction is the carboxy terminal direction, in accordance with standard usage and convention.

The terms “polypeptide”, “protein”, “fusion protein”, “chimeric protein”, “chimeric agent”, and “chimeric binding agent,” may be used interchangeably herein and generally refer to a polymer of amino acid residues. As described herein, “polypeptides”, “proteins”, “fusion proteins”, “chimeric proteins”, “chimeric agents”, or “chimeric binding agents” may be chains of amino acids whose alpha carbons may be linked through peptide bonds. The terminal amino acid at one end of the chain (e.g., amino terminal) therefore may have a free amino group, while the terminal amino acid at the other end of the chain (e.g., carboxy terminal) may have a free carboxyl group. As used herein, the term “amino terminus” (e.g., abbreviated N-terminus) generally refers to the free α-amino group on an amino acid at the amino terminal of a peptide or to the α-amino group (e.g., imino group when participating in a peptide bond) of an amino acid at any other location within the peptide. Similarly, the term “carboxy terminus” generally refers to the free carboxyl group on the carboxy terminus of a peptide or the carboxyl group of an amino acid at any other location within the peptide. Peptides and proteins also include essentially any polyamino acid including, but not limited to, peptide mimetics such as amino acids joined by an ether or thioether as opposed to an amide bond.

In some examples, a binding agent comprises an amino acid sequence that is identical to an amino acid sequence found in an endogenous immunoglobulin molecule (e.g., an endogenous Cε2 domain). As an alternative, a binding agent may comprise an amino acid sequence that has been varied, modified, or mutated compared to an amino acid sequence found in an endogenous immunoglobulin molecule (e.g., a Cε2 domain that comprises one or more amino acid variations).

The terms “comprising” and “having,” as used herein, may be used interchangeably. For example, the terms “comprising a sequence set forth in SEQ ID NO: 5” and “having a sequence set forth in SEQ ID NO: 5” may be used interchangeably herein.

Polypeptides, fusion or chimeric proteins of the present disclosure may include polypeptides, fusion or chimeric proteins that have been modified in any way to: (1) reduce susceptibility to proteolysis, (2) reduce susceptibility to oxidation, (3) alter binding affinity for forming molecular complexes (e.g., protein-receptor complexes), (4) alter binding affinities, and (5) confer or modify other physicochemical or functional properties. Single or multiple amino acid substitutions (e.g., conservative amino acid substitutions) may be made in a naturally occurring sequence (e.g., in the portion of the polypeptide outside the domain(s) forming intermolecular contacts). A “conservative amino acid substitution” generally refers to the substitution in a polypeptide of an amino acid with a functionally similar amino acid. The following six groups each contain amino acids that may be conservative substitutions for one another: i) Alanine (A), Serine (S), and Threonine (T); ii) Aspartic acid (D) and Glutamic acid (E); iii) Asparagine (N) and Glutamine (Q); iv) Arginine (R) and Lysine (K); v) Isoleucine (I), Leucine (L), Methionine (M), and Valine (V); vi) Phenylalanine (F), Tyrosine (Y), and Tryptophan (W).

The terms “selectivity”, “receptor selectivity”, and “preferential binding,” as used herein, may be used interchangeably and generally refer to the ability of a protein or fusion protein to bind a specific receptor or group of receptors. A protein or fusion protein of the present disclosure may bind a first specific receptor or group of receptors with a higher affinity compared to a second receptor or group of receptors. For example, a fusion protein with one or more amino acid variations compared to a wild-type sequence may have an increased (e.g., at least about 100, 150, 160, 166 or 200-fold increased) affinity for FcγRIIB (an inhibitory receptor) compared to FcεRI (an inhibitory receptor), and thus may preferentially bind to FcγRIIB compared to FcεRI.

The terms “polypeptide fragment”, “protein fragment”, and “truncated polypeptide,” as used herein, generally refer to a polypeptide or protein that has an amino-terminal and/or carboxy-terminal deletion as compared to a corresponding full-length polypeptide or protein. Amino acid fragments may be at least 5, at least 10, at least 25, at least 50, at least 100, at least 150, at least 200, at least 250, at least 300, at least 350, at least 400, at least 450, at least 500, at least 600, at least 700, at least 800, at least 900, or at least 1000 amino acids in length. Fragments may also be, e.g., at most 1000, at most 900, at most 800, at most 700, at most 600, at most 500, at most 450, at most 400, at most 350, at most 300, at most 250, at most 200, at most 150, at most 100, at most 50, at most 25, at most 10, or at most 5 amino acids in length. A fragment may further comprise, at either or both of its ends, one or more additional amino acids, a sequence of amino acids from a different naturally-occurring protein (e.g., an Fc or leucine zipper domain), or an artificial amino acid sequence (e.g., an artificial linker sequence).

The terms “polypeptide”, “protein”, “fusion protein”, “chimeric protein”, “chimeric agent”, or “chimeric binding agent” in conjunction with “variant”, “mutant”, “enriched mutant”, or “permuted enriched mutant,” as used herein, generally refer to a peptide or polypeptide that may comprise an amino acid sequence wherein one or more amino acid residues may be inserted into, deleted from and/or substituted into the amino acid sequence relative to another polypeptide sequence. The number of amino acid residues to be inserted, deleted, or substituted is at least 1, at least 2, at least 3, at least 4, at least 5, at least 10, at least 25, at least 50, at least 75, at least 100, at least 125, at least 150, at least 175, at least 200, at least 225, at least 250, at least 275, at least 300, at least 350, at least 400, at least 450, or at least 500 amino acids in length. Variants of the present disclosure include fusion molecules (e.g., peptide constructs).

A “derivative” of a molecule (e.g., a small molecule, polypeptide, or protein), as used herein, generally refers to a small molecule, polypeptide, or protein that may have been chemically modified, e.g., introduction of different functional groups or conjugation to another chemical moiety such as polyethylene glycol, albumin (e.g., human serum albumin), phosphorylation, and glycosylation.

The term “% sequence identity” may be used interchangeably herein with the term “% identity” and generally refers to the level of amino acid sequence identity between two or more peptide sequences or the level of nucleotide sequence identity between two or more nucleotide sequences, when aligned using a sequence alignment program. As used herein, 80% identity generally refers to the same thing as 80% sequence identity determined by a defined algorithm, and refers to a given sequence is at least 80% identical to another length of another sequence. The % identity may be selected from, e.g., at least 60%, at least 65%, at least 70%, at least 75%, at least 80%, at least 85%, at least 90%, at least 95%, or at least 99% or more sequence identity to a given sequence. The % identity is in the range of, e.g., about 60% to about 70%, about 70% to about 80%, about 80% to about 85%, about 85% to about 90%, about 90% to about 95%, or about 95% to about 99%. The term “% sequence similarity” may be used interchangeably herein with the term “% similarity” and generally refers to the level of amino acid sequence similarity between two or more peptide sequences or the level of nucleotide sequence identity between two or more nucleotide sequences, when aligned using a sequence alignment program. As used herein, 80% similarity generally refers to the same thing as 80% sequence similarity determined by a defined algorithm, and refers to a given sequence is at least 80% similar to another length of another sequence. The % similarity may be selected from, e.g., at least 60%, at least 65%, at least 70%, at least 75%, at least 80%, at least 85%, at least 90%, at least 95%, or at least 99% or more sequence similarity to a given sequence. The % similarity is in the range of, e.g., about 60% to about 70%, about 70% to about 80%, about 80% to about 85%, about 85% to about 90%, about 90% to about 95%, or about 95% to about 99%.

Amino acid variations as described herein may be abbreviated such that a substitution of a serine residue at a position 267 by a glutamic acid residue may be referred to as a “S267E variation.”

A protein or polypeptide of the present disclosure may be “substantially pure,” “substantially homogeneous”, or “substantially purified” when at least about 60% to 75% of a sample exhibits a single species of polypeptide. The polypeptide or protein may be monomeric or multimeric. A substantially pure polypeptide or protein may typically comprise about 50%, 60%, 70%, 80% or 90% W/W of a protein sample, more usually about 95%, and e.g., will be over 99% pure. Protein purity or homogeneity may be indicated in a number of ways, such as polyacrylamide gel electrophoresis of a protein sample, followed by visualizing a single polypeptide band upon staining the gel with a commonly used stain. For certain purposes, higher resolution is provided by using high-pressure liquid chromatography (e.g., HPLC) or other high-resolution analytical techniques (e.g., LC-mass spectrometry).

The terms “allergy”, “allergic reaction”, “allergic disease”, “allergic condition”, “atopy”, and “hypersensitivity reactions,” as used herein, may be used interchangeably.

The term “pharmaceutical composition,” as used herein, generally refers to a composition suitable for pharmaceutical use in a subject such as an animal (e.g., human or mouse). A pharmaceutical composition may comprise a pharmacologically effective amount of an active agent and a pharmaceutically acceptable carrier. The term “pharmacologically effective amount” generally refers to that amount of an agent effective to produce the intended biological or pharmacological result.

The term “pharmaceutically acceptable carrier,” as used herein, generally refers to any of the standard pharmaceutical carriers, vehicles, buffers, and excipients, such as a phosphate buffered saline solution, 5% aqueous solution of dextrose, and emulsions, such as an oil/water or water/oil emulsion, and various types of wetting agents and/or adjuvants. Suitable pharmaceutical carriers and formulations are described in Remington's Pharmaceutical Sciences, 21st Ed. 2005, Mack Publishing Co, Easton. A “pharmaceutically acceptable salt” may be a salt that may be formulated into a compound for pharmaceutical use including, e.g., metal salts (sodium, potassium, magnesium, calcium, etc.) and salts of ammonia or organic amines.

The terms “treat”, “treating” and “treatment,” as used herein, generally refer to a method of alleviating or abrogating a biological disorder (e.g., an allergic disease) and/or at least one of its attendant symptoms. As used herein, to “alleviate” a disease, disorder or condition generally refers to reducing the severity and/or occurrence frequency of the symptoms of the disease, disorder, or condition. Further, references herein to “treatment” may include references to curative, palliative, and prophylactic or diagnostic treatment.

Generally, a cell of the present disclosure may be a eukaryotic cell or a prokaryotic cell. A cell may be an animal cell or a plant cell. An animal cell may include a cell from a marine invertebrate, fish, insects, amphibian, reptile, or mammal. A mammalian cell may be obtained from a primate, ape, equine, bovine, porcine, canine, feline, or rodent. A mammal may be a primate, ape, dog, cat, rabbit, ferret, or the like. A rodent may be a mouse, rat, hamster, gerbil, hamster, chinchilla, or guinea pig. A bird cell may be from a canary, parakeet or parrots. A reptile cell may be from a turtles, lizard or snake. A fish cell may be from a tropical fish. The fish cell may be from a zebrafish (e.g., Danio rerio). A worm cell may be from a nematode (e.g., C. elegans). An amphibian cell may be from a frog. An arthropod cell may be from a tarantula or hermit crab.

A mammalian cell may also include cells obtained from a primate (e.g., a human or a non-human primate). A mammalian cell may include a blood cell, a stem cell, an epithelial cell, connective tissue cell, hormone secreting cell, a nerve cell, a skeletal muscle cell, or an immune system cell. The methods and compositions of the present disclosure may be used in combination with one or more mammalian blood cells.

The term “vector,” as used herein, generally refers to a DNA molecule capable of replication in a host cell and/or to which another DNA segment may be operatively linked so as to bring about replication of the attached segment. A plasmid is an exemplary vector.

The term “subject,” as used herein, generally refers to a human or to another animal. A subject may be of any age, a subject may be an infant, a toddler, a child, a pre-adolescent, an adolescent, an adult, or an elderly individual.

Ranges may be expressed herein as from “about” one particular value, and/or to “about” another particular value. When such a range is expressed, another aspect of the disclosure includes from the one particular value and/or to the other particular value. Similarly, when values are expressed as approximations, by use of the antecedent “about,” it will be understood that the particular value forms another aspect of the disclosure. It will be further understood that the endpoints of each of the ranges may be in relation to the other endpoint, and independently of the other endpoint. The term “about,” as used herein, refers to a range that is 15% plus or minus from a stated numerical value within the context of the particular usage. For example, about 10 may include a range from 8.5 to 11.5.

### Chimeric Binding Agents

The present disclosure provides methods and compositions comprising one or more chimeric binding agents for the prevention and or treatment of diseases, particularly allergic diseases and conditions. Allergies that may be prevented and/or treated with the presently described methods and compositions include IgE-mediated hypersensitivity (e.g., Type I), IgG-mediated cytotoxic hypersensitivity (e.g., Type II), immune complex-mediated hypersensitivity (e.g., Type III), and cell-mediated hypersensitivity (e.g., Type IV). The compositions as described herein may comprise chimeric binding agents comprising one or more regions comprising one or more functional domains of one or more immunoglobulin molecules (e.g., IgG or IgE). The compositions as described herein may be used to prevent and/or treat a disease or condition and comprise a region comprising one or more functional domains from or derived from an immunoglobulin E (IgE) molecule and a region comprising one or more functional domains from or derived from an immunoglobulin G (IgG) molecule. The compositions described herein may comprise a chimeric binding agent comprising a region derived from an Fc domain of an IgE molecule and a region derived from an Fc domain of an IgG molecule. Thus, the IgG-Fc-IgE-Fc proteins of the present disclosure may comprise two Fc domains from or derived from two different Ig molecules such as IgE Fc domain and an IgG Fc domain. As described herein, the terms “IgG-Fc-IgE-Fc protein”, “chimeric binding agent”, “fusion protein”, “fusion polypeptide” and “chimeric agent” may be used interchangeably.

Further provided herein are compositions and methods for suppression and/or inactivation of FceRI⁺ cells to induce durable and measurable anti-allergic effects in a subject suffering from allergy, particularly IgE-mediated allergies. One solution provided herein includes multivalent chimeric binding agents that are capable of binding FcεRIs on FceRI⁺ cells and co-ligation of neighboring FcγRIIBs on FcεRI⁺ cells, thereby providing long-lasting (e.g., several weeks or longer with a single administration) ant-inflammatory effects. The multivalent chimeric binding agents of the present disclosure can bind FcεRIs on FceRI⁺ cells such as mast cells or basophils with one or more IgE-Fc-derived binding portions, thereby inhibiting the activation of FcεRIs by IgE antibodies. Moreover, the use of IgE-Fc-derived binding portions and the lack of F(ab)₂ regions prevents FcεRI receptor cross-linking which further reduces activation/degranulation of mast cells and basophils. The multivalent chimeric binding agents provided herein can further comprise one or more IgG-Fc-derived binding portions enabling the binding agent to co-ligate with inhibitory (anti-inflammatory) FcγRs such as FcγRIIB Such co-ligation of FcεRI and FcγRIIB can induce immunoreceptor tyrosine-based inhibition motif (ITIM) signaling that suppresses and/or inactivates FceRI⁺ effector cells (e.g., mast cells, basophils, etc.). Thus, the multivalent chimeric binding agents provided herein allow for less frequent administration due to their long-lasting FcεRI/FcγRIM-mediated anti-inflammatory effects. Moreover, the binding agents herein show no or only very low immunogenicity due to a lack of complementary-determining regions (CDRs) and F(ab)₂ regions.

Further provided herein are chimeric binding agents capable of inducing effector cell depletion via antibody-dependent cell-mediated cytotoxicity (ADCC) and/or complement dependent cytotoxicity (CDC). In some cases, such chimeric binding agent can comprise an IgG-Fc region having one or more amino acid substitution(s), deletion(s), and/or addition(s) compared to a wild-type IgG region. In some cases, such one or more amino acid substitution(s) can include a glutamic acid to alanine (E to A) substitution. In such instances, the chimeric binding agent can comprise, consist essentially of, or consist of an amino acid sequence having at least about 80%, 85%, 90%, 95%, 97%, 99%, or 100% sequence identity to the amino acid sequence set forth in SEQ ID NO: 28. A chimeric binding agent that comprises or consists of the amino acid sequence set forth in SEQ ID NO: 28 comprises an E333A substitution compared to a binding agent comprised a non-modified, human IgG region.

In some embodiments, the compositions disclosed herein may be used to prevent and/or treat IgE-mediated (e.g., Type I) allergic diseases and conditions (e.g., atopy). Allergic diseases and conditions that may be treated using the herein described methods and compositions include, but are not limited to, sinusitis, allergic rhinitis, asthma, eczema, hives, food allergies, drug allergies, sting insect allergies, anaphylaxis, urticaria, angioedema (e.g., hereditary angioedema), allergic gastroenteropathy, and seasonal allergies such as hay fever. Seasonal allergies may be prevented and/or treated using a chimeric binding agent as disclosed herein. Treatment may result from binding of a chimeric binding agent to an FcR (e.g., an FcεR and/or an FcγR) which may prevent an endogenous ligand (such as Ig molecules and Ig-antigen complexes) of such a receptor from binding to such receptors. Thus, binding of a chimeric binding agent to an FcεR may prevent an endogenous IgE molecule such as those bound to an antigen from binding to their endogenous target such as an FcεR.

Diseases such as urticaria may be prevented and/or treated using a chimeric binding agent as disclosed herein by inhibiting allergen-induced degranulation via antagonistic binding of the FcεR-binding region of the chimeric protein to a FcεR such as FcεRI. Autoimmune diseases that may be mediated by crosslinking of FcεRIs by anti-FCER1A IgG molecules may be prevented and/or treated using the chimeric binding agents of the present disclosure and by blocking the binding of auto-reactive antibodies to receptors such as FcεRI. Thus, binding of a chimeric binding agent as described herein (e.g., IgG-Fc-IgE-Fc protein) to FcεRI may be sufficient to effectively suppress autoantibody-induced inflammation.

The present disclosure provides methods and compositions that may comprise one or more chimeric binding agents. A chimeric binding agent as described herein may be a fusion polypeptide or a fusion protein, or it may be a chemical conjugate wherein two or more amino acid domains or other molecular building blocks may be chemically conjugated, e.g., via the formation of amide bonds. A chimeric binding agent as described herein may comprise one or more regions or domains. The one or more regions or domains may be functional domains that are naturally occurring or non-naturally occurring. Naturally occurring regions or functional domains of the chimeric binding agent may include certain regions or functional domains found in immunoglobulin molecules such as a Fab (e.g., an antigen binding or variable fragments or variable domains) or an Fc region (e.g., constant domains) of an immunoglobulin molecule. Regions and/or functional domains of immunoglobulins may include regions and/or functional domains of the immunoglobulin isotypes immunoglobulin E (IgE), immunoglobulin G (IgG), immunoglobulin M (IgM), immunoglobulin A (IgA), and immunoglobulin D (IgD). The regions and/or functional domains of the chimeric binding agents may be from immunoglobulin subclasses such as the IgG subclasses IgG1, IgG2 IgG3, and IgG4. Immunoglobulin E domains that a chimeric binding agent of the present disclosure may be comprised of may include Cε1, Cε2, Cε3, and Cε4 domains. Immunoglobulin G domains that a chimeric binding agent of the present disclosure may be comprised of may include Cγ1, Cγ2, Cγ3 domains, and one or more hinge regions.

The chimeric binding agents of the present disclosure may comprise one or more regions (e.g., those with an amino acid sequence set forth in any one of SEQ ID NO: 3-SEQ ID NO: 6 or SEQ ID NO: 8-SEQ ID NO: 10) comprising one or more functional domains (e.g., Cε1, Cε2, Cε3, Cε4, Cγ1, Cγ2, Cγ3 and one or more hinge regions) from one or more immunoglobulin molecules. A chimeric binding agent may comprise wild-type amino acid sequences of one or more domains of the one or more immunoglobulin molecules. A wild-type domain of an IgE region as disclosed herein may be associated with the NCBI Accession No. AAB59395. A wild-type domain of an IgG region as disclosed herein may be associated with the NCBI Accession No. AXN93646. A chimeric binding agent comprising one or more domains may be derived from wild-type immunoglobulin molecules and thus may comprise one or more amino acid variations (e.g., amino acid variations such as S267E, L328F, and T396F). As described herein, amino acid variations may improve the pharmacokinetic and/or pharmacodynamic properties of a chimeric binding agent such as serum half-life, protease resistance, bioavailability, and receptor binding affinity.

The chimeric binding agents of the present disclosure may comprise one or more immunoglobulin domains (or functional domains) and/or derivatives, fragments, or variants thereof, or any combination thereof. As described herein, the terms “immunoglobulin domain” and “functional domain” may be used interchangeably.

The chimeric binding agents as disclosed herein may comprise one, two, three, or four or more functional domains from an immunoglobulin E molecule (e.g., an “IgE region” comprising one or more of Cε1, Cε2, Cε3, and/or Cε4 domains) and one, two, three, or four or more domains from an IgG (e.g., IgG1) molecule (e.g., an “IgG region” comprising one or more of Cγ1, Cγ2, Cγ3, and/or a hinge region), or any combination thereof, and wherein one or more or all of the functional domains may be derivatives, fragments, variants, or homologs of the respective wild-type functional domain (e.g., a functional domain that comprises one or more amino acid variations compared to the respective wild-type domain). A chimeric binding agent as disclosed herein may comprise three domains from or derived from an IgE molecule (e.g., wherein the IgE region comprises an amino acid sequence set forth in any one of SEQ ID NO: 3-SEQ ID NO: 6) and two domains from or derived from an IgG molecule (e.g., wherein the IgG region comprises an amino acid sequence set forth in any one of SEQ ID NO: 8-SEQ ID NO: 10). The Ig domains may be conjugated to, linked to, or fused to one another. The domains from or derived from an IgE molecule may be linked to two domains from or derived from an IgG molecule via a linker such as a flexible linker. A chimeric binding agent as disclosed herein may comprise the IgE Fc domains Cε2, Cε3, and Cε4, and the IgG Fc domains Cγ2 and Cγ3, or any derivative, fragment, variant, or homolog thereof, and wherein the connectivity between the functional domains is Cγ2-Cγ3-Cε2-Cε3-Cε4 (read from N-terminus to C-terminus). The IgG domains may be linked to the IgE domains via a linker. The linker may be an amino acid linker. The amino acid linker may comprise glycine and serine residues such as GGGGS (i.e., G₄S, SEQ ID NO: 13) or a derivative thereof, e.g., (G₄S)₃ (SEQ ID NO: 14). One or more of the IgG domains and one or more of the IgE domains may comprise one or more amino acid variations. The one or more amino acid variations may alter the binding affinity of the chimeric agent to one or more proteins (e.g., receptors such as Fc receptors), compared to an endogenous molecule comprising the IgG or IgE domain(s). Thus, a chimeric binding agent as disclosed herein may comprise an IgE region comprising an amino acid sequence set forth in any one of SEQ ID NO: 3-SEQ ID NO: 6, an IgG region comprising an amino acid sequence set forth in any one of SEQ ID NO: 8-SEQ ID NO: 10, and a linker having an amino acid sequence set forth in any one of SEQ ID NO: 13-SEQ ID NO: 19.

A chimeric binding agent as disclosed herein (e.g., an IgG-Fc-IgE-Fc fusion protein) may comprise an IgE region comprising an amino acid sequence set forth in any one of SEQ ID NO: 3-SEQ ID NO: 6, an IgG region comprising an amino acid sequence set forth in any one of SEQ ID NO: 8-SEQ ID NO: 10 having the connectivity Cγ2-Cγ3-Cε2-Cε3-Cε4, and a linker (connecting the IgG-Fc and IgE-Fc regions) having the amino acid sequence set forth in SEQ ID NO: 14, wherein the linker may connect the IgE region with the IgG region and may be located between the domains Cγ3 and Cε2. A chimeric binding agent may further comprise a hinge region to allow for flexibility and pairing of the single chains (e.g., two single chains). In other instances, a chimeric binding agent can have the connectivity Cε2-Cε3-Cε4-Cγ2-Cγ3, wherein the IgE-Fc region (Cε2-Cε3-Cε4) is connected to the IgG-Fc region (Cγ2-Cγ3) by a linker. Such linker can comprise an amino acid sequence. In such instances, the linker can have the amino acid sequence set forth in SEQ ID NO: 14. In other instances, the linker comprises or consists of an IgG-Fc hinge region.

The chimeric binding agents of the present disclosure may be capable of binding one or more molecules. The one or more molecules may be proteins such as enzymes or receptors. The chimeric binding agents of the present disclosure may be capable of binding to one or more receptors. The binding of a chimeric binding agent to the one or more receptors may be simultaneous. The one or more receptors that a chimeric binding agent may bind to may be Fc receptors such as Fc-gamma (Fcγ) receptors (FcγRs), Fc-epsilon (Fcε) receptors (FcεRs), or Fc-alpha (Fcα) receptors (FcαRs). FcγRs that a chimeric binding agent as described herein may bind to include FcγRI (CD64), FcγRIIA (CD32), FcγRIIB1 (CD32), FcγRIIB2 (CD32), FcγRIIIA (CD16a), and FcγRIIIB (CD16b). FcεRs that a chimeric binding agent may bind to include FcεRI (high-affinity Fcε receptor) and FcεRII (CD23 or low-affinity Fcε receptor). The Fc receptor that a chimeric binding agent of the present disclosure binds to may be a soluble Fc receptor or a membrane-bound Fc receptor (e.g., those Fc receptors located on the surface of cells such as immune cells including mast cells, basophils, eosinophils, or dendritic cells).

The chimeric binding agents of the present disclosure may comprise one or more IgE or IgE-derived functional domains and one or more IgG or IgG-derived functional domains and may bind simultaneously to an FcεR and an FcγR, respectively. Interaction of a chimeric binding agent with an Fc receptor may be activating (e.g., agonistic) or inhibitory (e.g., antagonistic). Thus, a chimeric binding agent comprising an FcεR binding region and an FcγR binding region may bind an FcεR in an antagonistic manner and an FcγR in an agonistic manner. The antagonistic binding of an FcεR and agonistic binding of an FcγR of a chimeric binding agent may occur simultaneously.

A chimeric binding agent comprising one or more IgE or IgE-derived functional domains and one or more IgG or IgG-derived functional domains may bind to FcεRI and an FcγRIIB (e.g., FcγRIIB1 and/or FcγRIIB2), respectively. Binding of a chimeric binding agent to one or more receptors may result in one or more biological effects. Binding of a chimeric binding agent comprising one or more IgE or IgE-derived functional domains to an FcεR (e.g., FcεRI) may suppress phosphorylation of immunoreceptor tyrosine-based activation motifs (ITAMs) of the FcεRI receptor's γ-chain and thus may suppress the signaling cascade causing cell degranulation. The FcεR binding region of the chimeric agent may thus act in an antagonistic manner when binding FcεRI. Furthermore, engagement and/or co-ligation of FcγRIIB using the chimeric binding agents as described herein may trigger immunoreceptor tyrosine-based inhibition motif phosphorylation which may induce a signaling pathway that results in inactivation and suppression of the effector cell (e.g., a mast cell or a basophil). Thus, the FcγR binding region of the chimeric agent may act in an agonistic manner when binding FcγRIIB and positively inactivating and thereby suppressing an effector cell (e.g., a mast cell or a basophil). The chimeric binding agents of the present disclosure may be referred to as IgG-Fc-IgE-Fc fusion proteins, indicating that the fusion protein comprises an IgE or IgE-derived FcεR binding region and an IgG or IgG-derived FcγR binding region. The IgG-Fc-IgE-Fc fusion proteins of the present disclosure may be capable of binding an FcεR and an FcγR simultaneously. Simultaneous binding of an FcεR (e.g., FcεRI) and an FcγR (e.g., FcγRIIB) may cause phosphorylation of immunoreceptor tyrosine-based inhibition motifs (ITIMs). Furthermore, binding of a chimeric binding agent to an FcR (e.g., an FcεR and/or an FcγR) may prevent an endogenous ligand (such as Ig molecules and Ig-antigen complexes) of such a receptor from binding to such receptors. Binding of a chimeric binding agent to an FcεR may prevent an endogenous IgE molecule such as those bound to an antigen from binding to an FcεR.

A chimeric binding agent as described herein may comprise one or more regions that have a binding specificity for one or more receptors, and wherein each of the one or more regions comprises one or more functional domains. A chimeric binding agent as disclosed herein may comprise a first region comprising one or more functional domains from or derived from an IgE and a second region comprising one or more functional domains from or derived from an IgG. A chimeric binding agent as disclosed herein may comprise a first region that has a binding specificity with at least one functional domain an immunoglobulin E (IgE) molecule, and a second binding region with at least one functional domain of an immunoglobulin G (IgG) molecule, and wherein the chimeric binding agent binds to an FcεR, thereby blocking the FcεR. A chimeric binding agent as disclosed herein may comprise a first region that has a binding specificity for an FcεRI with at least one functional domain of an immunoglobulin E (IgE) molecule, and a second binding region that has a binding specificity for an FcγRIIB with at least one functional domain of an immunoglobulin G (IgG) molecule, and wherein the chimeric binding agent binds to an FcεRI and an FcγRIIB, thereby blocking the FcεR and activating the FcγRIIB (e.g., causing phosphorylation of ITIMs). The binding of a chimeric binding agent to an FcεRI and an FcγRII may occur simultaneously.

The chimeric binding agents of the present disclosure may be used to treat and/or prevent a disorder or a condition in a subject (e.g., a human or a mouse). The disorder may be an inflammatory disorder and/or may involve inflammation. Thus, the chimeric binding agents of the present disclosure may be used to suppress, reduce, inhibit, or eliminate inflammation in a subject (e.g., a human or a mouse). The chimeric binding agents of the present disclosure may suppress, reduce, inhibit, or eliminate inflammation in a subject by inhibiting cell degranulation such as degranulation of a mast cell or a basophil or any other immune cell. Based on their mechanism, the chimeric binding agents of the present disclosure may be particularly useful to treat, prevent, or diagnose IgE-mediated inflammatory diseases such as allergies in a subject (e.g., a human or a mouse).

The present disclosure provides chimeric binding agents (e.g., IgG-Fc-IgE-Fc fusion proteins) comprising one or more region comprising one or more functional (e.g., immunoglobulin) domains from and/or derived from (for instance when comprising one or more amino acid residue variations) one or more immunoglobulin molecules. A chimeric binding agent as described herein may comprise a first and second region, wherein the first region comprises one or more functional domains from and/or derived from an IgE molecule, and the second region comprises one or more functional domains from and/or derived from an IgG molecule. Such chimeric binding agent may bind to an FcεR, an FcγR, or both receptors simultaneously. The chimeric binding agents of the present disclosure may bind to FcεRI in an antagonistic manner, and to FcγRIIB in an agonistic manner, thereby inhibiting cellular degranulation of mast cells and/or basophils. Thus, the chimeric binding agents of the present disclosure may be used to suppress, reduce, inhibit, or eliminate inflammation in a subject (e.g., a human or a mouse). The chimeric binding agents of the present disclosure may suppress, reduce, inhibit, or eliminate inflammation in a subject by inhibiting cell degranulation such as degranulation of a mast cell or a basophil. Based on their mechanism, the chimeric binding agents of the present disclosure may be particularly useful to treat, prevent, or diagnose IgE-mediated inflammatory diseases in a subject (e.g., a human or a mouse).

A chimeric binding agent as described herein may comprise one or more regions that have a binding specificity for one or more receptors, and wherein each of the one or more regions comprises one or more functional domains. A chimeric binding agent as disclosed herein may comprise a first region comprising one or more functional domains from or derived from an IgE and a second region comprising one or more functional domains from or derived from an IgG. A chimeric binding agent as disclosed herein may comprise a first region that has a binding specificity with at least one functional domain of an immunoglobulin E (IgE) molecule, and a second binding region with at least one functional domain of an immunoglobulin G (IgG) molecule, and wherein the chimeric binding agent binds to an FcεR, thereby blocking the FcεR. A chimeric binding agent as disclosed herein may comprise a first region that has a binding specificity with at least one functional domain an immunoglobulin E (IgE) molecule, and a second binding region with at least one functional domain of an immunoglobulin G (IgG) molecule, and wherein the chimeric binding agent binds to an FcεRI and an FcγRIIB, thereby blocking the FcεR and activating the FcγRIIB (e.g., causing phosphorylation of ITIM). The binding of a chimeric binding agent as described herein to an FcεRI and an FcγRII may occur simultaneously.

Regions and/or functional domains of immunoglobulins may include regions and/or functional domains of the immunoglobulin isotypes immunoglobulin E (IgE), immunoglobulin G (IgG), immunoglobulin M (IgM), immunoglobulin A (IgA), and immunoglobulin D (IgD). The regions and/or functional domains of the chimeric binding agents may be from immunoglobulin subclasses such as the IgG subclasses IgG1, IgG2 IgG3, and IgG4. Immunoglobulin E domains that a chimeric binding agent of the present disclosure may be comprised of may include Cε1, Cε2, Cε3, and Cε4 domains. Immunoglobulin G (e.g., IgG1) domains that a chimeric binding agent of the present disclosure may be comprised of may include Cγ1, Cγ2, Cγ3 domains and a hinge region.

The chimeric binding agents of the present disclosure may comprise a first region comprising one or more functional domains from or derived from an IgE molecule, and a second region comprising one or more functional domains from or derived from an IgG molecule.

General structure of IgE and IgE-Fc molecules. IgE molecules share similar molecular and structural characteristics of other Ig molecules, with two heavy chains and two light chains. However, the heavy ε-chain of IgE may comprise one more domain than the heavy γ-chain of IgG. The Cε3 and Cε4 domains of IgE may be functionally and structurally similar to the Cγ2 and Cγ3 domains of IgG. The two Cε2 domains, which may be a distinguishing feature of an IgE molecule, may be present in place of the flexible hinge region generally found in IgG molecules. The Cε2 domains may fold back and make contact with the Cε3 and Cε4 domains, presumably acting as a spacer region between the Fab (e.g., the antigen-binding fragment) arms and the Fc (crystallizable fragment) region, wherein the compact, bent Cε2 domains may provide considerable flexibility in the conformation of IgE. Moreover, an IgE-Fc region herein can comprise or consist of Cε2, Cε3 and Cε4 domains. In some cases, IgE core glycan(s) can provide binding of an IgE-Fc region of a binding agent to FcεRs, such as an FcεRI, and can also provide increased inhibition of degranulation of target cells, such as mast cells and basophils. Moreover, in some cases, α-2,6-sialylation of an IgE-Fc portion can reduce the binding affinity of the IgE-Fc molecule to FcεRs, such as an FcεRI. In other cases, fucosylation of an IgE-Fc portion may not affect binding of the IgE-Fc molecule to FcεRs, such as an FcεRI. An IgE-Fc region of a binding agent herein can provide efficient and fast binding of the IgE-Fc region to FcεRs, such as an FcεRI. In such cases, an IgE-Fc region can bind to FcεRs in a manner such that at least about 50%, 75%, 90%, or 99% of IgE-Fc molecules have bound to FcεRs within at most about 60 min, 30 min, 15 min, or 5 mins.

General IgG structure. Similar to IgE molecules, IgG molecules may generally comprise two heavy chains and two light chains. The Fc region comprises a Cγ2 domain and a Cγ3 domain of each heavy chain, wherein each of the two Fab (e.g., the antigen binding fragment) regions comprises a variable and constant domain from each the heavy (Vγ and Cγ1) and the light (V_{L} and C_{L}) chain, resulting in a total of four domains per Fab region.

TABLE 1 shows exemplary nucleotide and amino acid sequences for chimeric binding agents as disclosed herein.

TABLE 1
Exemplary Nucleotide and Amino Acid Sequences

| SEQ ID NO | Sequence |
|---|---|
| SEQ ID NO: 1 | GTCTGCTCCAGGGACTTCACCCCGCC |
| | CACCGTGAAGATCTTACAGTCGTCCT |
| | GCGACGGCGGCGGGCACTTCCCCCCG |
| | ACCATCCAGCTCCTGTGCCTCGTCTC |
| | TGGGTACACCCCAGGGACTATCAACA |
| | TCACCTGGCTGGAGGACGGGCAGGTC |
| | ATGGACGTGGACTTGTCCACCGCCTC |
| | TACCACGCAGGAGGGTGAGCTGGCCT |
| | CCACACAAAGCGAGCTCACCCTCAGC |
| | CAGAAGCACTGGCTGTCAGACCGCAC |
| | CTACACCTGCCAGGTCACCTATCAAG |
| | GTCACACCTTTGAGGACAGCACCAAG |
| | AAGTGTGCAGATTCCAACCCGAGAGG |
| | GGTGAGCGCCTACCTAAGCCGGCCCA |
| | GCCCGTTCGACCTGTTCATCCGCAAG |
| | TCGCCCACGATCACCTGTCTGGTGGT |
| | GGACCTGGCACCCAGCAAGGGGACCG |
| | TGAACCTGACCTGGTCCCGGGCCAGT |
| | GGGAAGCCTGTGAACCACTCCACCAG |
| | AAAGGAGGAGAAGCAGCGCAATGGCA |
| | CGTTAACCGTCACGTCCACCCTGCCG |
| | GTGGGCACCCGAGACTGGATCGAGGG |
| | GGAGACCTACCAGTGCAGGGTGACCC |
| | ACCCCCACCTGCCCAGGGCCCTCATG |
| | CGGTCCACGACCAAGACCAGCGGCCC |
| | GCGTGCTGCCCCGGAAGTCTATGCGT |
| | TTGCGACGCCGGAGTGGCCGGGGAGC |
| | CGGGACAAGCGCACCCTCGCCTGCCT |
| | GATCCAGAACTTCATGCCTGAGGACA |
| | TCTCGGTGCAGTGGCTGCACAACGAG |
| | GTGCAGCTCCCGGACGCCCGGCACAG |
| | CACGACGCAGCCCCGCAAGACCAAGG |
| | GCTCCGGCTTCTTCGTCTTCAGCCGC |
| | CTGGAGGTGACCAGGGCCGAATGGGA |
| | GCAGAAAGATGAGTTCATCTGCCGTG |
| | CAGTCCATGAGGCAGCGAGCCCCTCA |
| | CAGACCGTCCAGCGAGCGGTGTCTGT |
| | AAATCCCGGTAAA |
| SEQ ID NO: 2 | ATGGTTAGATCTGTTCGACCTGTCAA |
| | CATCACTGAGCCCACCTTGGAGCTAC |
| | TCCATTCATCCTGCGACCCCAATGCA |
| | TTCCACTCCACCATCCAGCTGTACTG |
| | CTTCATTTATGGCCACATCCTAAATG |
| | ATGTCTCTGTCAGCTGGCTAATGGAC |
| | GATCGGGAGATAACTGATACACTTGC |
| | ACAAACTGTTCTAATCAAGGAGGAAG |
| | GCAAACTAGCCTCTACCTGCAGTAAA |
| | CTCAACATCACTGAGCAGCAATGGAT |
| | GTCTGAAAGCACCTTCACCTGCAAGG |
| | TCACCTCCCAAGGCGTAGACTATTTG |
| | GCCCACACTCGGAGATGCCCAGATCA |
| | TGAGCCACGGGGTGTGATTACCTACC |
| | TGATCCCACCCAGCCCCCTGGACCTG |
| | TATCAAAACGGTGCTCCCAAGCTTAC |
| | CTGTCTGGTGGTGGACCTGGAAAGCG |
| | AGAAGAATGTCAATGTGACGTGGAAC |
| | CAAGAGAAGAAGACTTCAGTCTCAGC |
| | ATCCCAGTGGTACACTAAGCACCACA |
| | ATAACGCCACAACTAGTATCACCTCC |
| | ATCCTGCCTGTAGTTGCCAAGGACTG |
| | GATTGAAGGCTACGGCTATCAGTGCA |
| | TAGTGGACCACCCTGATTTTCCCAAG |
| | CCCATTGTGCGTTCCATCACCAAGAC |
| | CCCAGGCCAGCGCTCAGCCCCCGAGG |
| | TATATGTGTTCCCACCACCAGAGGAG |
| | GAGAGCGAGGACAAACGCACACTCAC |
| | CTGTTTGATCCAGAACTTCTTCCCTG |
| | AGGATATCTCTGTGCAGTGGCTGGGG |
| | GATGGCAAACTGATCTCAAACAGCCA |
| | GCACAGTACCACAACACCCCTGAAAT |
| | CCAATGGCTCCAATCAAGGCTTCTTC |
| | ATCTTCAGTCGCCTAGAGGTCGCCAA |
| | GACACTCTGGACACAGAGAAAACAGT |
| | TCACCTGCCAAGTGATCCATGAGGCA |
| | CTTCAGAAACCCAGGAAACTGGAGAA |
| | AACAATATCCACAAGCCTTGGTAACA |
| | CCTCCCTCCGTCCCTCCTAG |
| SEQ ID NO: 3 | TVSGAWAKQMFTCRVAHTPSSTDWVD |
| | NKTFSVCSRDFTPPTVKILQSSCDGG |
| | GHFPPTIQLLCLVSGYTPGTINITWL |
| | EDGQVMDVDLSTASTTQEGELASTQS |
| | ELTLSQKHWLSDRTYTCQVTYQGHTF |
| | EDSTKKCADSNPRGVSAYLSRPSPFD |
| | LFIRKSPTITCLVVDLAPSKGTVNLT |
| | WSRASGKPVNHSTRKEEKQRNGTLTV |
| | TSTLPVGTRDWIEGETYQCRVTHPHL |
| | PRALMRSTTKTSGPRAAPEVYAFATP |
| | EWPGSRDKRTLACLIQNFMPEDISVQ |
| | WLHNEVQLPDARHSTTQPRKTKGSGF |
| | FVFSRLEVTRAEWEQKDEFICRAVHE |
| | AASPSQTVQRAVSVNPGK |
| SEQ ID NO: 4 | TVSGAWAKQMFTCRVAHTPSSTDWVD |
| | NKTFSVCSRDFTPPTVKILQSSCDGG |
| | GHFPPTIQLLCLVSGYTPGTINITWL |
| | EDGQVMDVDLSTASTTQEGELASTQS |
| | ELTLSQKHWLSDRTYTCQVTYQGHTF |
| | EDSTKKCADSNPRGVSAYLSRPSPFD |
| | LFIRKSPTITCLVVDLAPSKGTVNLT |
| | WSRASGKPVNHSTRKEEKQRNGALTV |
| | TSTLPVGTRDWIEGETYQCRVTHPHL |
| | PRALMRSTTKTSGPRAAPEVYAFATP |
| | EWPGSRDKRTLACLIQNFMPEDISVQ |
| | WLHNEVQLPDARHSTTQPRKTKGSGF |
| | FVFSRLEVTRAEWEQKDEFICRAVHE |
| | AASPSQTVQRAVSVNPGK |
| SEQ ID NO: 5 | MVRSVRPVNITEPTLELLHSSCDPNA |
| | FHSTIQLYCFIYGHILNDVSVSWLMD |
| | DREITDTLAQTVLIKEEGKLASTCSK |
| | LNITEQQWMSESTFTCKVTSQGVDYL |
| | AHTRRCPDHEPRGVITYLIPPSPLDL |
| | YQNGAPKLTCLVVDLESEKNVNVTWN |
| | QEKKTSVSASQWYTKHHNNATTSITS |
| | ILPVVAKDWIEGYGYQCIVDHPDFPK |
| | PIVRSITKTPGQRSAPEVYVFPPPEE |
| | ESEDKRTLTCLIQNFFPEDISVQWLG |
| | DGKLISNSQHSTTTPLKSNGSNQGFF |
| | IFSRLEVAKTLWTQRKQFTCQVIHEA |
| | LQKPRKLEKTISTSLGNTSLRPS |
| SEQ ID NO: 6 | VRPVNITEPTLELLHSSCDPNAFHST |
| | IQLYCFIYGHILNDVSVSWLMDDREI |
| | TDTLAQTVLIKEEGKLASTCSKLNIT |
| | EQQWMSESTFTCKVTSQGVDYLAHTR |
| | RCPDHEPRGVITYLIPPSPLDLYQNG |
| | APKLTCLVVDLESEKNVNVTWNQEKK |
| | TSVSASQWYTKHHNNATTSITSILPV |
| | VAKDWIEGYGYQCIVDHPDFPKPIVR |
| | SITKTPGQRSAPEVYVFPPPEEESED |
| | KRTLTCLIQNFFPEDISVQWLGDGKL |
| | ISNSQHSTTTPLKSNGSNQGFFIFSR |
| | LEVAKTLWTQRKQFTCQVIHEALQKP |
| | RKLEKTISTSLGNTSLRPS |
| SEQ ID NO: 7 | TGACAAAACTCACACATGCCCACCGT |
| | GCCCAGCACCTGAACTCCTGGGGGGA |
| | CCGTCAGTCTTCCTCTTCCCCCCAAA |
| | ACCCAAGGACACCCTCATGATCTCCC |
| | GGACCCCTGAGGTCACATGCGTGGTG |
| | GTGGACGTGAGCCACGAAGACCCTGA |
| | GGTCAAGTTCAACTGGTACGTGGACG |
| | GCGTGGAGGTGCATAATGCCAAGACA |
| | AAGCCGCGGGAGGAGCAGTACAACAG |
| | CACGTACCGTGTGGTCAGCGTCCTCA |
| | CCGTCCTGCACCAGGACTGGCTGAAT |
| | GGCAAGGAGTACAAGTGCAAGGTCTC |
| | CAACAAAGCCCTCCCAGCCCCCATCG |
| | AGAAAACCATCTCCAAAGCCAAAGGG |
| | CAGCCCCGAGAACCACAGGTGTACAC |
| | CCTGCCCCCATCCCGGGATGAGCTGA |
| | CCAAGAACCAGGTCAGCCTGACCTGC |
| | CTGGTCAAAGGCTTCTATCCCAGCGA |
| | CATCGCCGTGGAGTGGGAGAGCAATG |
| | GGCAGCCGGAGAACAACTACAAGACC |
| | ACGCCTCCCGTGCTGGACTCCGACGG |
| | CTCCTTCTTCCTCTACAGCAAGCTCA |
| | CCGTGGACAAGAGCAGGTGGCAGCAG |
| | GGGAACGTCTTCTCATGCTCCGTGAT |
| | GCATGAGGCTCTGCACAACCACTACA |
| | CGCAGAAGAGCCTCTCCCTGTCTCCG |
| | GGTAAA |
| SEQ ID NO: 8 | DKTHTCPPCPAPELLGGPSVFLFPPK |
| | PKDTLMISRTPEVTCVVVDVSHEDPE |
| | VKFNWYVDGVEVHNAKTKPREEQYNS |
| | TYRVVSVLTVLHQDWLNGKEYKCKVS |
| | NKALPAPIEKTISKAKGQPREPQVYT |
| | LPPSRDELTKNQVSLTCLVKGFYPSD |
| | IAVEWESNGQPENNYKTTPPVLDSDG |
| | SFFLYSKLTVDKSRWQQGNVFSCSVM |
| | HEALHNHYTQKSLSLSPGK |
| SEQ ID NO: 9 | MVRSDKTHTCPPCPAPELLGGPSVFL |
| | FPPKPKDTLMISRTPEVTCVVVDVEH |
| | EDPEVKFNWYVDGVEVHNAKTKPREE |
| | QYNSTYRVVSVLTVLHQDWLNGKEYK |
| | CKVSNKAFPAPIEKTISKAKGQPREP |
| | QVYTLPPSRDELTKNQVSLTCLVKGF |
| | YPSDIAVEWESNGQPENNYKTTPPVL |
| | DSDGSFFLYSKLTVDKSRWQQGNVFS |
| | CSVMHEALHNHYTQKSLSLSPGK |
| SEQ ID NO: 10 | MVRSDKTHTCPPCPAPELLGGPSVFL |
| | FPPKPKDTLMISRTPEVTCVVVDVEH |
| | EDPEVKFNWYVDGVEVHNAKTKPREE |
| | QYNSTYRVVSVLTVLHQDWLNGKEYK |
| | CKVSNKAFPAPIEKTISKAKGQPREP |
| | QVYTLPPSREEMTKNQVSLTCLVKGF |
| | YPSDIAVEWESNGQPENNYKTTPPVL |
| | DSDGSFFLYSKLTVDKSRWQQGNVFS |
| | CSVMHEALHNHYTQKSLSLSPGKGS |
| SEQ ID NO: 11 | ATGGTTAGATCTGACAAAACTCACAC |
| | ATGCCCACCGTGCCCAGCACCTGAAC |
| | TCCTGGGGGGACCGTCAGTCTTCCTC |
| | TTCCCCCCAAAACCCAAGGACACCCT |
| | CATGATCTCCCGGACCCCTGAGGTCA |
| | CATGCGTGGTGGTGGACGTGGAACAC |
| | GAAGACCCTGAGGTCAAGTTCAACTG |
| | GTACGTGGACGGCGTGGAGGTGCATA |
| | ATGCCAAGACAAAGCCGCGGGAGGAG |
| | CAGTACAACAGCACGTACCGTGTGGT |
| | CAGCGTCCTCACCGTCCTGCACCAGG |
| | ACTGGCTGAATGGCAAGGAGTACAAG |
| | TGCAAGGTCTCCAACAAAGCCTTCCC |
| | AGCCCCCATCGAGAAAACCATCTCCA |
| | AAGCCAAAGGGCAGCCCCGAGAACCA |
| | CAGGTGTACACCCTGCCCCCATCCCG |
| | GGAGGAGATGACCAAGAACCAGGTCA |
| | GCCTGACCTGCCTGGTCAAAGGCTTC |
| | TATCCCAGCGACATCGCCGTGGAGTG |
| | GGAGAGCAATGGGCAGCCGGAGAACA |
| | ACTACAAGACCACGCCTCCCGTGCTG |
| | GACTCCGACGGCTCCTTCTTCCTCTA |
| | CAGCAAGCTCACCGTGGACAAGAGCA |
| | GGTGGCAGCAGGGGAACGTCTTCTCA |
| | TGCTCCGTGATGCACGAGGCTCTGCA |
| | CAACCACTACACGCAGAAGAGCCTCT |
| | CCCTGTCTCCGGGTAAAGGATCCGGC |
| | GGCGGAGGCTCCGGCGGCGGAGGCTC |
| | CGGCGGCGGAGGCTCCGTTCGACCTG |
| | TCAACATCACTGAGCCCACCTTGGAG |
| | CTACTCCATTCATCCTGCGACCCCAA |
| | TGCATTCCACTCCACCATCCAGCTGT |
| | ACTGCTTCATTTATGGCCACATCCTA |
| | AATGATGTCTCTGTCAGCTGGCTAAT |
| | GGACGATCGGGAGATAACTGATACAC |
| | TTGCACAAACTGTTCTAATCAAGGAG |
| | GAAGGCAAACTAGCCTCTACCTGCAG |
| | TAAACTCAACATCACTGAGCAGCAAT |
| | GGATGTCTGAAAGCACCTTCACCTGC |
| | AAGGTCACCTCCCAAGGCGTAGACTA |
| | TTTGGCCCACACTCGGAGATGCCCAG |
| | ATCATGAGCCACGGGGTGTGATTACC |
| | TACCTGATCCCACCCAGCCCCCTGGA |
| | CCTGTATCAAAACGGTGCTCCCAAGC |
| | TTACCTGTCTGGTGGTGGACCTGGAA |
| | AGCGAGAAGAATGTCAATGTGACGTG |
| | GAACCAAGAGAAGAAGACTTCAGTCT |
| | CAGCATCCCAGTGGTACACTAAGCAC |
| | CACAATAACGCCACAACTAGTATCAC |
| | CTCCATCCTGCCTGTAGTTGCCAAGG |
| | ACTGGATTGAAGGCTACGGCTATCAG |
| | TGCATAGTGGACCACCCTGATTTTCC |
| | CAAGCCCATTGTGCGTTCCATCACCA |
| | AGACCCCAGGCCAGCGCTCAGCCCCC |
| | GAGGTATATGTGTTCCCACCACCAGA |
| | GGAGGAGAGCGAGGACAAACGCACAC |
| | TCACCTGTTTGATCCAGAACTTCTTC |
| | CCTGAGGATATCTCTGTGCAGTGGCT |
| | GGGGGATGGCAAACTGATCTCAAACA |
| | GCCAGCACAGTACCACAACACCCCTG |
| | AAATCCAATGGCTCCAATCAAGGCTT |
| | CTTCATCTTCAGTCGCCTAGAGGTCG |
| | CCAAGACACTCTGGACACAGAGAAAA |
| | CAGTTCACCTGCCAAGTGATCCATGA |
| | GGCACTTCAGAAACCCAGGAAACTGG |
| | AGAAAACAATATCCACAAGCCTTGGT |
| | AACACCTCCCTCCGTCCCTCCTAG |
| SEQ ID NO: 12 | MVRSDKTHTCPPCPAPELLGGPSVF |
| (Compound A) | LFPPKPKDTLMISRTPEVTCVVVDV |
| | EHEDPEVKFNWYVDGVEVHNAKTKP |
| | REEQYNSTYRVVSVLTVLHQDWLNG |
| | KEYKCKVSNKAFPAPIEKTISKAKG |
| | QPREPQVYTLPPSREEMTKNQVSLT |
| | CLVKGFYPSDIAVEWESNGQPENNY |
| | KTTPPVLDSDGSFFLYSKLTVDKSR |
| | WQQGNVFSCSVMHEALHNHYTQKSL |
| | SLSPGKGSGGGGSGGGGSGGGGSVR |
| | PVNITEPTLELLHSSCDPNAFHSTI |
| | QLYCFIYGHILNDVSVSWLMDDREI |
| | TDTLAQTVLIKEEGKLASTCSKLNI |
| | TEQQWMSESTFTCKVTSQGVDYLAH |
| | TRRCPDHEPRGVITYLIPPSPLDLY |
| | QNGAPKLTCLVVDLESEKNVNVTWN |
| | QEKKTSVSASQWYTKHHNNATTSIT |
| | SILPVVAKDWIEGYGYQCIVDHPDF |
| | PKPIVRSITKTPGQRSAPEVYVFPP |
| | PEEESEDKRTLTCLIQNFFPEDISV |
| | QWLGDGKLISNSQHSTTTPLKSNGS |
| | NQGFFIFSRLEVAKTLWTQRKQFTC |
| | QVIHEALQKPRKLEKTISTSLGNTS |
| | LRPS |
| SEQ ID NO: 20 | GGATCTGCGATCGCTCCGGTGCCCG |
| | TCAGTGGGCAGAGCGCACATCGCCC |
| | ACAGTCCCCGAGAAGTTGGGGGGAG |
| | GGGTCGGCAATTGAACGGGTGCCTA |
| | GAGAAGGTGGCGCGGGGTAAACTGG |
| | GAAAGTGATGTCGTGTACTGGCTCC |
| | GCCTTTTTCCCGAGGGTGGGGGAGA |
| | ACCGTATATAAGTGCAGTAGTCGCC |
| | GTGAACGTTCTTTTTCGCAACGGGT |
| | TTGCCGCCAGAACACAGCTGAAGCT |
| | TCGAGGGGCTCGCATCTCTCCTTCA |
| | CGCGCCCGCCGCCCTACCTGAGGCC |
| | GCCATCCACGCCGGTTGAGTCGCGT |
| | TCTGCCGCCTCCCGCCTGTGGTGCC |
| | TCCTGAACTGCGTCCGCCGTCTAGG |
| | TAAGTTTAAAGCTCAGGTCGAGACC |
| | GGGCCTTTGTCCGGCGCTCCCTTGG |
| | AGCCTACCTAGACTCAGCCGGCTCT |
| | CCACGCTTTGCCTGACCCTGCTTGC |
| | TCAACTCTACGTCTTTGTTTCGTTT |
| | TCTGTTCTGCGCCGTTACAGATCCA |
| | AGCTGTGACCGGCGCCTACCTGAGA |
| | TCACCGGCGAAGGAGGGCCACCATG |
| | TACAGGATGCAACTCCTGTCTTGCA |
| | TTGCACTAAGTCTTGCACTTGTCAC |
| | GAATTCGATATCTCGAGTGGTCTGC |
| | TCCAGGGACTTCACCCCGCCCACCG |
| | TGAAGATCTTACAGTCGTCCTGCGA |
| | CGGCGGCGGGCACTTCCCCCCGACC |
| | ATCCAGCTCCTGTGCCTCGTCTCTG |
| | GGTACACCCCAGGGACTATCAACAT |
| | CACCTGGCTGGAGGACGGGCAGGTC |
| | ATGGACGTGGACTTGTCCACCGCCT |
| | CTACCACGCAGGAGGGTGAGCTGGC |
| | CTCCACACAAAGCGAGCTCACCCTC |
| | AGCCAGAAGCACTGGCTGTCAGACC |
| | GCACCTACACCTGCCAGGTCACCTA |
| | TCAAGGTCACACCTTTGAGGACAGC |
| | ACCAAGAAGTGTGCAGATTCCAACC |
| | CGAGAGGGGTGAGCGCCTACCTAAG |
| | CCGGCCCAGCCCGTTCGACCTGTTC |
| | ATCCGCAAGTCGCCCACGATCACCT |
| | GTCTGGTGGTGGACCTGGCACCCAG |
| | CAAGGGGACCGTGAACCTGACCTGG |
| | TCCCGGGCCAGTGGGAAGCCTGTGA |
| | ACCACTCCACCAGAAAGGAGGAGAA |
| | GCAGCGCAATGGCACGTTAACCGTC |
| | ACGTCCACCCTGCCGGTGGGCACCC |
| | GAGACTGGATCGAGGGGGAGACCTA |
| | CCAGTGCAGGGTGACCCACCCCCAC |
| | CTGCCCAGGGCCCTCATGCGGTCCA |
| | CGACCAAGACCAGCGGCCCGCGTGC |
| | TGCCCCGGAAGTCTATGCGTTTGCG |
| | ACGCCGGAGTGGCCGGGGAGCCGGG |
| | ACAAGCGCACCCTCGCCTGCCTGAT |
| | CCAGAACTTCATGCCTGAGGACATC |
| | TCGGTGCAGTGGCTGCACAACGAGG |
| | TGCAGCTCCCGGACGCCCGGCACAG |
| | CACGACGCAGCCCCGCAAGACCAAG |
| | GGCTCCGGCTTCTTCGTCTTCAGCC |
| | GCCTGGAGGTGACCAGGGCCGAATG |
| | GGAGCAGAAAGATGAGTTCATCTGC |
| | CGTGCAGTCCATGAGGCAGCGAGCC |
| | CCTCACAGACCGTCCAGCGAGCGGT |
| | GTCTGTAAATCCCGGTAAAAGATCT |
| | GACAAAACTCACACATGCCCACCGT |
| | GCCCAGCACCTGAACTCCTGGGGGG |
| | ACCGTCAGTCTTCCTCTTCCCCCCA |
| | AAACCCAAGGACACCCTCATGATCT |
| | CCCGGACCCCTGAGGTCACATGCGT |
| | GGTGGTGGACGTGAGCCACGAAGAC |
| | CCTGAGGTCAAGTTCAACTGGTACG |
| | TGGACGGCGTGGAGGTGCATAATGC |
| | CAAGACAAAGCCGCGGGAGGAGCAG |
| | TACAACAGCACGTACCGTGTGGTCA |
| | GCGTCCTCACCGTCCTGCACCAGGA |
| | CTGGCTGAATGGCAAGGAGTACAAG |
| | TGCAAGGTCTCCAACAAAGCCCTCC |
| | CAGCCCCCATCGAGAAAACCATCTC |
| | CAAAGCCAAAGGGCAGCCCCGAGAA |
| | CCACAGGTGTACACCCTGCCCCCAT |
| | CCCGGGAGGAGATGACCAAGAACCA |
| | GGTCAGCCTGACCTGCCTGGTCAAA |
| | GGCTTCTATCCCAGCGACATCGCCG |
| | TGGAGTGGGAGAGCAATGGGCAGCC |
| | GGAGAACAACTACAAGACCACGCCT |
| | CCCGTGCTGGACTCCGACGGCTCCT |
| | TCTTCCTCTACAGCAAGCTCACCGT |
| | GGACAAGAGCAGGTGGCAGCAGGGG |
| | AACGTCTTCTCATGCTCCGTGATGC |
| | ACGAGGCTCTGCACAACCACTACAC |
| | GCAGAAGAGCCTCTCCCTGTCTCCG |
| | GGTAAATGAGTGCTAGCTGGCCAGA |
| | CATGATAAGATACATTGATGAGTTT |
| | GGACAAACCACAACTAGAATGCAGT |
| | GAAAAAAATGCTTTATTTGTGAAAT |
| | TTGTGATGCTATTGCTTTATTTGTA |
| | ACCATTATAAGCTGCAATAAACAAG |
| | TTAACAACAACAATTGCATTCATTT |
| | TATGTTTCAGGTTCAGGGGGAGGTG |
| | TGGGAGGTTTTTTAAAGCAAGTAAA |
| | ACCTCTACAAATGTGGTATGGAATT |
| | AATTCTAAAATACAGCATAGCAAAA |
| | CTTTAACCTCCAAATCAAGCCTCTA |
| | CTTGAATCCTTTTCTGAGGGATGAA |
| | TAAGGCATAGGCATCAGGGGCTGTT |
| | GCCAATGTGCATTAGCTGTTTGCAG |
| | CCTCACCTTCTTTCATGGAGTTTAA |
| | GATATAGTGTATTTTCCCAAGGTTT |
| | GAACTAGCTCTTCATTTCTTTATGT |
| | TTTAAATGCACTGACCTCCCACATT |
| | CCCTTTTTAGTAAAATATTCAGAAA |
| | TAATTTAAATACATCATTGCAATGA |
| | AAATAAATGTTTTTTATTAGGCAGA |
| | ATCCAGATGCTCAAGGCCCTTCATA |
| | ATATCCCCCAGTTTAGTAGTTGGAC |
| | TTAGGGAACAAAGGAACCTTTAATA |
| | GAAATTGGACAGCAAGAAAGCGAGC |
| | TTCTAGCTTATCCTCAGTCCTGCTC |
| | CTCTGCCACAAAGTGCACGCAGTTG |
| | CCGGCCGGGTCGCGCAGGGCGAACT |
| | CCCGCCCCCACGGCTGCTCGCCGAT |
| | CTCGGTCATGGCCGGCCCGGAGGCG |
| | TCCCGGAAGTTCGTGGACACGACCT |
| | CCGACCACTCGGCGTACAGCTCGTC |
| | CAGGCCGCGCACCCACACCCAGGCC |
| | AGGGTGTTGTCCGGCACCACCTGGT |
| | CCTGGACCGCGCTGATGAACAGGGT |
| | CACGTCGTCCCGGACCACACCGGCG |
| | AAGTCGTCCTCCACGAAGTCCCGGG |
| | AGAACCCGAGCCGGTCGGTCCAGAA |
| | CTCGACCGCTCCGGCGACGTCGCGC |
| | GCGGTGAGCACCGGAACGGCACTGG |
| | TCAACTTGGCCATGATGGCTCCTCC |
| | TGTCAGGAGAGGAAAGAGAAGAAGG |
| | TTAGTACAATTGCTATAGTGAGTTG |
| | TATTATACTATGCAGATATACTATG |
| | CCAATGATTAATTGTCAAACTAGGG |
| | CTGCAGGGTTCATAGTGCCACTTTT |
| | CCTGCACTGCCCCATCTCCTGCCCA |
| | CCCTTTCCCAGGCATAGACAGTCAG |
| | TGACTTACCAAACTCACAGGAGGGA |
| | GAAGGCAGAAGCTTGAGACAGACCC |
| | GCGGGACCGCCGAACTGCGAGGGGA |
| | CGTGGCTAGGGCGGCTTCTTTTATG |
| | GTGCGCCGGCCCTCGGAGGCAGGGC |
| | GCTCGGGGAGGCCTAGCGGCCAATC |
| | TGCGGTGGCAGGAGGCGGGGCCGAA |
| | GGCCGTGCCTGACCAATCCGGAGCA |
| | CATAGGAGTCTCAGCCCCCCGCCCC |
| | AAAGCAAGGGGAAGTCACGCGCCTG |
| | TAGCGCCAGCGTGTTGTGAAATGGG |
| | GGCTTGGGGGGGTTGGGGCCCTGAC |
| | TAGTCAAAACAAACTCCCATTGACG |
| | TCAATGGGGTGGAGACTTGGAAATC |
| | CCCGTGAGTCAAACCGCTATCCACG |
| | CCCATTGATGTACTGCCAAAACCGC |
| | ATCATCATGGTAATAGCGATGACTA |
| | ATACGTAGATGTACTGCCAAGTAGG |
| | AAAGTCCCATAAGGTCATGTACTGG |
| | GCATAATGCCAGGCGGGCCATTTAC |
| | CGTCATTGACGTCAATAGGGGGCGT |
| | ACTTGGCATATGATACACTTGATGT |
| | ACTGCCAAGTGGGCAGTTTACCGTA |
| | AATACTCCACCCATTGACGTCAATG |
| | GAAAGTCCCTATTGGCGTTACTATG |
| | GGAACATACGTCATTATTGACGTCA |
| | ATGGGCGGGGGTCGTTGGGCGGTCA |
| | GCCAGGCGGGCCATTTACCGTAAGT |
| | TATGTAACGCCTGCAGGTTAATTAA |
| | GAACATGTGAGCAAAAGGCCAGCAA |
| | AAGGCCAGGAACCGTAAAAAGGCCG |
| | CGTTGCTGGCGTTTTTCCATAGGCT |
| | CCGCCCCCCTGACGAGCATCACAAA |
| | AATCGACGCTCAAGTCAGAGGTGGC |
| | GAAACCCGACAGGACTATAAAGATA |
| | CCAGGCGTTTCCCCCTGGAAGCTCC |
| | CTCGTGCGCTCTCCTGTTCCGACCC |
| | TGCCGCTTACCGGATACCTGTCCGC |
| | CTTTCTCCCTTCGGGAAGCGTGGCG |
| | CTTTCTCATAGCTCACGCTGTAGGT |
| | ATCTCAGTTCGGTGTAGGTCGTTCG |
| | CTCCAAGCTGGGCTGTGTGCACGAA |
| | CCCCCCGTTCAGCCCGACCGCTGCG |
| | CCTTATCCGGTAACTATCGTCTTGA |
| | GTCCAACCCGGTAAGACACGACTTA |
| | TCGCCACTGGCAGCAGCCACTGGTA |
| | ACAGGATTAGCAGAGCGAGGTATGT |
| | AGGCGGTGCTACAGAGTTCTTGAAG |
| | TGGTGGCCTAACTACGGCTACACTA |
| | GAAGAACAGTATTTGGTATCTGCGC |
| | TCTGCTGAAGCCAGTTACCTTCGGA |
| | AAAAGAGTTGGTAGCTCTTGATCCG |
| | GCAAACAAACCACCGCTGGTAGCGG |
| | TGGTTTTTTTGTTTGCAAGCAGCAG |
| | ATTACGCGCAGAAAAAAAGGATCTC |
| | AAGAAGATCCTTTGATCTTTTCTAC |
| | GGGGTCTGACGCTCAGTGGAACGAA |
| | AACTCACGTTAAGGGATTTTGGTCA |
| | TGGCTAGTTAATTAACATTTAAATC |
| | AGCGGCCGCAATAAAATATCTTTAT |
| | TTTCATTACATCTGTGTGTTGGTTT |
| | TTTGTGTGAATCGTAACTAACATAC |
| | GCTCTCCATCAAAACAAAACGAAAC |
| | AAAACAAACTAGCAAAATAGGCTGT |
| | CCCCAGTGCAAGTGCAGGTGCCAGA |
| | ACATTTCTCTATCGAA |
| SEQ ID NO: 21 | MYRMQLLSCIALSLALVTNSISRVV |
| | CSRDETPPTVKILQSSCDGGGHFPP |
| | TIQLLCLVSGYTPGTINITWLEDGQ |
| | VMDVDLSTASTTQEGELASTQSELT |
| | LSQKHWLSDRTYTCQVTYQGHTFED |
| | STKKCADSNPRGVSAYLSRPSPFDL |
| | FIRKSPTITCLVVDLAPSKGTVNLT |
| | WSRASGKPVNHSTRKEEKQRNGTLT |
| | VTSTLPVGTRDWIEGETYQCRVTHP |
| | HLPRALMRSTTKTSGPRAAPEVYAF |
| | ATPEWPGSRDKRTLACLIQNFMPED |
| | ISVQWLHNEVQLPDARHSTTQPRKT |
| | KGSGFFVFSRLEVTRAEWEQKDEFI |
| | CRAVHEAASPSQTVQRAVSVNPGKR |
| | SDKTHTCPPCPAPELLGGPSVFLFP |
| | PKPKDTLMISRTPEVTCVVVDVSHE |
| | DPEVKFNWYVDGVEVHNAKTKPREE |
| | QYNSTYRVVSVLTVLHQDWLNGKEY |
| | KCKVSNKALPAPIEKTISKAKGQPR |
| | EPQVYTLPPSREEMTKNQVSLTCLV |
| | KGFYPSDIAVEWESNGQPENNYKTT |
| | PPVLDSDGSFFLYSKLTVDKSRWQQ |
| | GNVFSCSVMHEALHNHYTQKSLSLS |
| | PGK |
| SEQ ID NO: 22 | GGATCTGCGATCGCTCCGGTGCCCG |
| | TCAGTGGGCAGAGCGCACATCGCCC |
| | ACAGTCCCCGAGAAGTTGGGGGGAG |
| | GGGTCGGCAATTGAACGGGTGCCTA |
| | GAGAAGGTGGCGCGGGGTAAACTGG |
| | GAAAGTGATGTCGTGTACTGGCTCC |
| | GCCTTTTTCCCGAGGGTGGGGGAGA |
| | ACCGTATATAAGTGCAGTAGTCGCC |
| | GTGAACGTTCTTTTTCGCAACGGGT |
| | TTGCCGCCAGAACACAGCTGAAGCT |
| | TCGAGGGGCTCGCATCTCTCCTTCA |
| | CGCGCCCGCCGCCCTACCTGAGGCC |
| | GCCATCCACGCCGGTTGAGTCGCGT |
| | TCTGCCGCCTCCCGCCTGTGGTGCC |
| | TCCTGAACTGCGTCCGCCGTCTAGG |
| | TAAGTTTAAAGCTCAGGTCGAGACC |
| | GGGCCTTTGTCCGGCGCTCCCTTGG |
| | AGCCTACCTAGACTCAGCCGGCTCT |
| | CCACGCTTTGCCTGACCCTGCTTGC |
| | TCAACTCTACGTCTTTGTTTCGTTT |
| | TCTGTTCTGCGCCGTTACAGATCCA |
| | AGCTGTGACCGGCGCCTACCTGAGA |
| | TCACCGGTGAATTCGATATCTCGAG |
| | CACCATGGTCTGCTCCAGGGACTTC |
| | ACCCCGCCCACCGTGAAGATCTTAC |
| | AGTCGTCCTGCGACGGCGGCGGGCA |
| | CTTCCCCCCGACCATCCAGCTCCTG |
| | TGCCTCGTCTCTGGGTACACCCCAG |
| | GGACTATCAACATCACCTGGCTGGA |
| | GGACGGGCAGGTCATGGACGTGGAC |
| | TTGTCCACCGCCTCTACCACGCAGG |
| | AGGGTGAGCTGGCCTCCACACAAAG |
| | CGAGCTCACCCTCAGCCAGAAGCAC |
| | TGGCTGTCAGACCGCACCTACACCT |
| | GCCAGGTCACCTATCAAGGTCACAC |
| | CTTTGAGGACAGCACCAAGAAGTGT |
| | GCAGATTCCAACCCGAGAGGGGTGA |
| | GCGCCTACCTAAGCCGGCCCAGCCC |
| | GTTCGACCTGTTCATCCGCAAGTCG |
| | CCCACGATCACCTGTCTGGTGGTGG |
| | ACCTGGCACCCAGCAAGGGGACCGT |
| | GAACCTGACCTGGTCCCGGGCCAGT |
| | GGGAAGCCTGTGAACCACTCCACCA |
| | GAAAGGAGGAGAAGCAGCGCAATGG |
| | CACGTTAACCGTCACGTCCACCCTG |
| | CCGGTGGGCACCCGAGACTGGATCG |
| | AGGGGGAGACCTACCAGTGCAGGGT |
| | GACCCACCCCCACCTGCCCAGGGCC |
| | CTCATGCGGTCCACGACCAAGACCA |
| | GCGGCCCGCGTGCTGCCCCGGAAGT |
| | CTATGCGTTTGCGACGCCGGAGTGG |
| | CCGGGGAGCCGGGACAAGCGCACCC |
| | TCGCCTGCCTGATCCAGAACTTCAT |
| | GCCTGAGGACATCTCGGTGCAGTGG |
| | CTGCACAACGAGGTGCAGCTCCCGG |
| | ACGCCCGGCACAGCACGACGCAGCC |
| | CCGCAAGACCAAGGGCTCCGGCTTC |
| | TTCGTCTTCAGCCGCCTGGAGGTGA |
| | CCAGGGCCGAATGGGAGCAGAAAGA |
| | TGAGTTCATCTGCCGTGCAGTCCAT |
| | GAGGCAGCGAGCCCCTCACAGACCG |
| | TCCAGCGAGCGGTGTCTGTAAATCC |
| | CGGTAAAAGATCTGACAAAACTCAC |
| | ACATGCCCACCGTGCCCAGCACCTG |
| | AACTCCTGGGGGGACCGTCAGTCTT |
| | CCTCTTCCCCCCAAAACCCAAGGAC |
| | ACCCTCATGATCTCCCGGACCCCTG |
| | AGGTCACATGCGTGGTGGTGGACGT |
| | GAGCCACGAAGACCCTGAGGTCAAG |
| | TTCAACTGGTACGTGGACGGCGTGG |
| | AGGTGCATAATGCCAAGACAAAGCC |
| | GCGGGAGGAGCAGTACAACAGCACG |
| | TACCGTGTGGTCAGCGTCCTCACCG |
| | TCCTGCACCAGGACTGGCTGAATGG |
| | CAAGGAGTACAAGTGCAAGGTCTCC |
| | AACAAAGCCCTCCCACTCCCCGAGG |
| | AGAAAACCATCTCCAAAGCCAAAGG |
| | GCAGCCCCGAGAACCACAGGTGTAC |
| | ACCCTGCCCCCATCCCGGGAGGAGA |
| | TGACCAAGAACCAGGTCAGCCTGAC |
| | CTGCCTGGTCAAAGGCTTCTATCCC |
| | AGCGACATCGCCGTGGAGTGGGAGA |
| | GCAATGGGCAGCCGGAGAACAACTA |
| | CAAGACCACGCCTCCCGTGCTGGAC |
| | TCCGACGGCTCCTTCTTCCTCTACA |
| | GCAAGCTCACCGTGGACAAGAGCAG |
| | GTGGCAGCAGGGGAACGTCTTCTCA |
| | TGCTCCGTGATGCACGAGGCTCTGC |
| | ACAACCACTACACGCAGAAGAGCCT |
| | CTCCCTGTCTCCGGGTAAATGAGTG |
| | CTAGCTGGCCAGACATGATAAGATA |
| | CATTGATGAGTTTGGACAAACCACA |
| | ACTAGAATGCAGTGAAAAAAATGCT |
| | TTATTTGTGAAATTTGTGATGCTAT |
| | TGCTTTATTTGTAACCATTATAAGC |
| | TGCAATAAACAAGTTAACAACAACA |
| | ATTGCATTCATTTTATGTTTCAGGT |
| | TCAGGGGGAGGTGTGGGAGGTTTTT |
| | TAAAGCAAGTAAAACCTCTACAAAT |
| | GTGGTATGGAATTAATTCTAAAATA |
| | CAGCATAGCAAAACTTTAACCTCCA |
| | AATCAAGCCTCTACTTGAATCCTTT |
| | TCTGAGGGATGAATAAGGCATAGGC |
| | ATCAGGGGCTGTTGCCAATGTGCAT |
| | TAGCTGTTTGCAGCCTCACCTTCTT |
| | TCATGGAGTTTAAGATATAGTGTAT |
| | TTTCCCAAGGTTTGAACTAGCTCTT |
| | CATTTCTTTATGTTTTAAATGCACT |
| | GACCTCCCACATTCCCTTTTTAGTA |
| | AAATATTCAGAAATAATTTAAATAC |
| | ATCATTGCAATGAAAATAAATGTTT |
| | TTTATTAGGCAGAATCCAGATGCTC |
| | AAGGCCCTTCATAATATCCCCCAGT |
| | TTAGTAGTTGGACTTAGGGAACAAA |
| | GGAACCTTTAATAGAAATTGGACAG |
| | CAAGAAAGCGAGCTTCTAGCTTATC |
| | CTCAGTCCTGCTCCTCTGCCACAAA |
| | GTGCACGCAGTTGCCGGCCGGGTCG |
| | CGCAGGGCGAACTCCCGCCCCCACG |
| | GCTGCTCGCCGATCTCGGTCATGGC |
| | CGGCCCGGAGGCGTCCCGGAAGTTC |
| | GTGGACACGACCTCCGACCACTCGG |
| | CGTACAGCTCGTCCAGGCCGCGCAC |
| | CCACACCCAGGCCAGGGTGTTGTCC |
| | GGCACCACCTGGTCCTGGACCGCGC |
| | TGATGAACAGGGTCACGTCGTCCCG |
| | GACCACACCGGCGAAGTCGTCCTCC |
| | ACGAAGTCCCGGGAGAACCCGAGCC |
| | GGTCGGTCCAGAACTCGACCGCTCC |
| | GGCGACGTCGCGCGCGGTGAGCACC |
| | GGAACGGCACTGGTCAACTTGGCCA |
| | TGATGGCTCCTCCTGTCAGGAGAGG |
| | AAAGAGAAGAAGGTTAGTACAATTG |
| | CTATAGTGAGTTGTATTATACTATG |
| | CAGATATACTATGCCAATGATTAAT |
| | TGTCAAACTAGGGCTGCAGGGTTCA |
| | TAGTGCCACTTTTCCTGCACTGCCC |
| | CATCTCCTGCCCACCCTTTCCCAGG |
| | CATAGACAGTCAGTGACTTACCAAA |
| | CTCACAGGAGGGAGAAGGCAGAAGC |
| | TTGAGACAGACCCGCGGGACCGCCG |
| | AACTGCGAGGGGACGTGGCTAGGGC |
| | GGCTTCTTTTATGGTGCGCCGGCCC |
| | TCGGAGGCAGGGCGCTCGGGGAGGC |
| | CTAGCGGCCAATCTGCGGTGGCAGG |
| | AGGCGGGGCCGAAGGCCGTGCCTGA |
| | CCAATCCGGAGCACATAGGAGTCTC |
| | AGCCCCCCGCCCCAAAGCAAGGGGA |
| | AGTCACGCGCCTGTAGCGCCAGCGT |
| | GTTGTGAAATGGGGGCTTGGGGGGG |
| | TTGGGGCCCTGACTAGTCAAAACAA |
| | ACTCCCATTGACGTCAATGGGGTGG |
| | AGACTTGGAAATCCCCGTGAGTCAA |
| | ACCGCTATCCACGCCCATTGATGTA |
| | CTGCCAAAACCGCATCATCATGGTA |
| | ATAGCGATGACTAATACGTAGATGT |
| | ACTGCCAAGTAGGAAAGTCCCATAA |
| | GGTCATGTACTGGGCATAATGCCAG |
| | GCGGGCCATTTACCGTCATTGACGT |
| | CAATAGGGGGCGTACTTGGCATATG |
| | ATACACTTGATGTACTGCCAAGTGG |
| | GCAGTTTACCGTAAATACTCCACCC |
| | ATTGACGTCAATGGAAAGTCCCTAT |
| | TGGCGTTACTATGGGAACATACGTC |
| | ATTATTGACGTCAATGGGCGGGGGT |
| | CGTTGGGCGGTCAGCCAGGCGGGCC |
| | ATTTACCGTAAGTTATGTAACGCCT |
| | GCAGGTTAATTAAGAACATGTGAGC |
| | AAAAGGCCAGCAAAAGGCCAGGAAC |
| | CGTAAAAAGGCCGCGTTGCTGGCGT |
| | TTTTCCATAGGCTCCGCCCCCCTGA |
| | CGAGCATCACAAAAATCGACGCTCA |
| | AGTCAGAGGTGGCGAAACCCGACAG |
| | GACTATAAAGATACCAGGCGTTTCC |
| | CCCTGGAAGCTCCCTCGTGCGCTCT |
| | CCTGTTCCGACCCTGCCGCTTACCG |
| | GATACCTGTCCGCCTTTCTCCCTTC |
| | GGGAAGCGTGGCGCTTTCTCATAGC |
| | TCACGCTGTAGGTATCTCAGTTCGG |
| | TGTAGGTCGTTCGCTCCAAGCTGGG |
| | CTGTGTGCACGAACCCCCCGTTCAG |
| | CCCGACCGCTGCGCCTTATCCGGTA |
| | ACTATCGTCTTGAGTCCAACCCGGT |
| | AAGACACGACTTATCGCCACTGGCA |
| | GCAGCCACTGGTAACAGGATTAGCA |
| | GAGCGAGGTATGTAGGCGGTGCTAC |
| | AGAGTTCTTGAAGTGGTGGCCTAAC |
| | TACGGCTACACTAGAAGAACAGTAT |
| | TTGGTATCTGCGCTCTGCTGAAGCC |
| | AGTTACCTTCGGAAAAAGAGTTGGT |
| | AGCTCTTGATCCGGCAAACAAACCA |
| | CCGCTGGTAGCGGTGGTTTTTTTGT |
| | TTGCAAGCAGCAGATTACGCGCAGA |
| | AAAAAAGGATCTCAAGAAGATCCTT |
| | TGATCTTTTCTACGGGGTCTGACGC |
| | TCAGTGGAACGAAAACTCACGTTAA |
| | GGGATTTTGGTCATGGCTAGTTAAT |
| | TAACATTTAAATCAGCGGCCGCAAT |
| | AAAATATCTTTATTTTCATTACATC |
| | TGTGTGTTGGTTTTTTGTGTGAATC |
| | GTAACTAACATACGCTCTCCATCAA |
| | AACAAAACGAAACAAAACAAACTAG |
| | CAAAATAGGCTGTCCCCAGTGCAAG |
| | TGCAGGTGCCAGAACATTTCTCTAT |
| | CGAA |
| SEQ ID NO: 23 | MVCSRDFTPPTVKILQSSCDGGGHF |
| | PPTIQLLCLVSGYTPGTINITWLED |
| | GQVMDVDLSTASTTQEGELASTQSE |
| | LTLSQKHWLSDRTYTCQVTYQGHTF |
| | EDSTKKCADSNPRGVSAYLSRPSPF |
| | DLFIRKSPTITCLVVDLAPSKGTVN |
| | LTWSRASGKPVNHSTRKEEKQRNGT |
| | LTVTSTLPVGTRDWIEGETYQCRVT |
| | HPHLPRALMRSTTKTSGPRAAPEVY |
| | AFATPEWPGSRDKRTLACLIQNEMP |
| | EDISVQWLHNEVQLPDARHSTTQPR |
| | KTKGSGEFVFSRLEVTRAEWEQKDE |
| | FICRAVHEAASPSQTVQRAVSVNPG |
| | KRSDKTHTCPPCPAPELLGGPSVFL |
| | EPPKPKDTLMISRTPEVTCVVVDVS |
| | HEDPEVKFNWYVDGVEVHNAKTKPR |
| | EEQYNSTYRVVSVLTVLHQDWLNGK |
| | EYKCKVSNKALPLPEEKTISKAKGQ |
| | PREPQVYTLPPSREEMTKNQVSLTC |
| | LVKGFYPSDIAVEWESNGQPENNYK |
| | TTPPVLDSDGSFFLYSKLTVDKSRW |
| | QQGNVFSCSVMHEALHNHYTQKSLS |
| | LSPGK |
| SEQ ID NO: 24 | CCTGCAGGGCCTGAAATAACCTCTG |
| | AAAGAGGAACTTGGTTAGGTACCTT |
| | CTGAGGCGGAAAGAACCAGCTGTGG |
| | AATGTGTGTCAGTTAGGGTGTGGAA |
| | AGTCCCCAGGCTCCCCAGCAGGCAG |
| | AAGTATGCAAAGCATGCATCTCAAT |
| | TAGTCAGCAACCAGGTGTGGAAAGT |
| | CCCCAGGCTCCCCAGCAGGCAGAAG |
| | TATGCAAAGCATGCATCTCAATTAG |
| | TCAGCAACCATAGTCCCACTAGTGG |
| | AGCCGAGAGTAATTCATACAAAAGG |
| | AGGGATCGCCTTCGCAAGGGGAGAG |
| | CCCAGGGACCGTCCCTAAATTCTCA |
| | CAGACCCAAATCCCTGTAGCCGCCC |
| | CACGACAGCGCGAGGAGCATGCGCT |
| | CAGGGCTGAGCGCGGGGAGAGCAGA |
| | GCACACAAGCTCATAGACCCTGGTC |
| | GTGGGGGGGAGGACCGGGGAGCTGG |
| | CGCGGGGCAAACTGGGAAAGCGGTG |
| | TCGTGTGCTGGCTCCGCCCTCTTCC |
| | CGAGGGTGGGGGAGAACGGTATATA |
| | AGTGCGGCAGTCGCCTTGGACGTTC |
| | TTTTTCGCAACGGGTTTGCCGTCAG |
| | AACGCAGGTGAGGGGCGGGTGTGGC |
| | TTCCGCGGGCCGCCGAGCTGGAGGT |
| | CCTGCTCCGAGCGGGCCGGGCCCCG |
| | CTGTCGTCGGCGGGGATTAGCTGCG |
| | AGCATTCCCGCTTCGAGTTGCGGGC |
| | GGCGCGGGAGGCAGAGTGCGAGGCC |
| | TAGCGGCAACCCCGTAGCCTCGCCT |
| | CGTGTCCGGCTTGAGGCCTAGCGTG |
| | GTGTCCGCGCCGCCGCCGCGTGCTA |
| | CTCCGGCCGCACTCTGGTCTTTTTT |
| | TTTTTTGTTGTTGTTGCCCTGCTGC |
| | CTTCGATTGCCGTTCAGCAATAGGG |
| | GCTAACAAAGGGAGGGTGCGGGGCT |
| | TGCTCGCCCGGAGCCCGGAGAGGTC |
| | ATGGTTGGGGAGGAATGGAGGGACA |
| | GGAGTGGCGGCTGGGGCCCGCCCGC |
| | CTTCGGAGCACATGTCCGACGCCAC |
| | CTGGATGGGGCGAGGCCTGGGGTTT |
| | TTCCCGAAGCAACCAGGCTGGGGTT |
| | AGCGTGCCGAGGCCATGTGGCCCCA |
| | GCACCCGGCACGATCTGGCTTGGCG |
| | GCGCCGCGTTGCCCTGCCTCCCTAA |
| | CTAGGGTGAGGCCATCCCGTCCGGC |
| | ACCAGTTGCGTGCGTGGAAAGATGG |
| | CCGCTCCCGGGCCCTGTTGCAAGGA |
| | GCTCAAAATGGAGGACGCGGCAGCC |
| | CGGTGGAGCGGGCGGGTGAGTCACC |
| | CACACAAAGGAAGAGGGCCTGGTCC |
| | CTCACCGGCTGCTGCTTCCTGTGAC |
| | CCCGTGGTCCTATCGGCCGCAATAG |
| | TCACCTCGGGCTTTTGAGCACGGCT |
| | AGTCGCGGCGGGGGGAGGGGATGTA |
| | ATGGCGTTGGAGTTTGTTCACATTT |
| | GGTGGGTGGAGACTAGTCAGGCCAG |
| | CCTGGCGCTGGAAGTCATTTTTGGA |
| | ATTTGTCCCCTTGAGTTTTGAGCGG |
| | AGCTAATTCTCGGGCTTCTTAGCGG |
| | TTCAAAGGTATCTTTTAAACCCTTT |
| | TTTAGGTGTTGTGAAAACCACCGCT |
| | AATTCAAAGCAACCGGTCACCATGG |
| | TTAGATCTGACAAAACTCACACATG |
| | CCCACCGTGCCCAGCACCTGAACTC |
| | CTGGGGGGACCGTCAGTCTTCCTCT |
| | TCCCCCCAAAACCCAAGGACACCCT |
| | CATGATCTCCCGGACCCCTGAGGTC |
| | ACATGCGTGGTGGTGGACGTGGAAC |
| | ACGAAGACCCTGAGGTCAAGTTCAA |
| | CTGGTACGTGGACGGCGTGGAGGTG |
| | CATAATGCCAAGACAAAGCCGCGGG |
| | AGGAGCAGTACAACAGCACGTACCG |
| | TGTGGTCAGCGTCCTCACCGTCCTG |
| | CACCAGGACTGGCTGAATGGCAAGG |
| | AGTACAAGTGCAAGGTCTCCAACAA |
| | AGCCTTCCCAGCCCCCATCGAGAAA |
| | ACCATCTCCAAAGCCAAAGGGCAGC |
| | CCCGAGAACCACAGGTGTACACCCT |
| | GCCCCCATCCCGGGAGGAGATGACC |
| | AAGAACCAGGTCAGCCTGACCTGCC |
| | TGGTCAAAGGCTTCTATCCCAGCGA |
| | CATCGCCGTGGAGTGGGAGAGCAAT |
| | GGGCAGCCGGAGAACAACTACAAGA |
| | CCACGCCTCCCGTGCTGGACTCCGA |
| | CGGCTCCTTCTTCCTCTACAGCAAG |
| | CTCACCGTGGACAAGAGCAGGTGGC |
| | AGCAGGGGAACGTCTTCTCATGCTC |
| | CGTGATGCACGAGGCTCTGCACAAC |
| | CACTACACGCAGAAGAGCCTCTCCC |
| | TGTCTCCGGGTAAAGGATCCGGCGG |
| | CGGAGGCTCCGGCGGCGGAGGCTCC |
| | GGCGGCGGAGGCTCCGTTCGACCTG |
| | TCAACATCACTGAGCCCACCTTGGA |
| | GCTACTCCATTCATCCTGCGACCCC |
| | AATGCATTCCACTCCACCATCCAGC |
| | TGTACTGCTTCATTTATGGCCACAT |
| | CCTAAATGATGTCTCTGTCAGCTGG |
| | CTAATGGACGATCGGGAGATAACTG |
| | ATACACTTGCACAAACTGTTCTAAT |
| | CAAGGAGGAAGGCAAACTAGCCTCT |
| | ACCTGCAGTAAACTCAACATCACTG |
| | AGCAGCAATGGATGTCTGAAAGCAC |
| | CTTCACCTGCAAGGTCACCTCCCAA |
| | GGCGTAGACTATTTGGCCCACACTC |
| | GGAGATGCCCAGATCATGAGCCACG |
| | GGGTGTGATTACCTACCTGATCCCA |
| | CCCAGCCCCCTGGACCTGTATCAAA |
| | ACGGTGCTCCCAAGCTTACCTGTCT |
| | GGTGGTGGACCTGGAAAGCGAGAAG |
| | AATGTCAATGTGACGTGGAACCAAG |
| | AGAAGAAGACTTCAGTCTCAGCATC |
| | CCAGTGGTACACTAAGCACCACAAT |
| | AACGCCACAACTAGTATCACCTCCA |
| | TCCTGCCTGTAGTTGCCAAGGACTG |
| | GATTGAAGGCTACGGCTATCAGTGC |
| | ATAGTGGACCACCCTGATTTTCCCA |
| | AGCCCATTGTGCGTTCCATCACCAA |
| | GACCCCAGGCCAGCGCTCAGCCCCC |
| | GAGGTATATGTGTTCCCACCACCAG |
| | AGGAGGAGAGCGAGGACAAACGCAC |
| | ACTCACCTGTTTGATCCAGAACTTC |
| | TTCCCTGAGGATATCTCTGTGCAGT |
| | GGCTGGGGGATGGCAAACTGATCTC |
| | AAACAGCCAGCACAGTACCACAACA |
| | CCCCTGAAATCCAATGGCTCCAATC |
| | AAGGCTTCTTCATCTTCAGTCGCCT |
| | AGAGGTCGCCAAGACACTCTGGACA |
| | CAGAGAAAACAGTTCACCTGCCAAG |
| | TGATCCATGAGGCACTTCAGAAACC |
| | CAGGAAACTGGAGAAAACAATATCC |
| | ACAAGCCTTGGTAACACCTCCCTCC |
| | GTCCCTCCGAAAATCTTTATTTTCA |
| | AGGTCATCATCATCATCATCATCAT |
| | TAGGCTAGCTGGCCAGACATGATAA |
| | GATACATTGATGAGTTTGGACAAAC |
| | CACAACTAGAATGCAGTGAAAAAAA |
| | TGCTTTATTTGTGAAATTTGTGATG |
| | CTATTGCTTTATTTGTAACCATTAT |
| | AAGCTGCAATAAACAAGTTAACAAC |
| | AACAATTGCATTCATTTTATGTTTC |
| | AGGTTCAGGGGGAGGTGTGGGAGGT |
| | TTTTTAAAGCAAGTAAAACCTCTAC |
| | AAATGTGGTATGGAAATGTTAATTA |
| | ACTAGCCATGACCAAAATCCCTTAA |
| | CGTGAGTTTTCGTTCCACTGAGCGT |
| | CAGACCCCGTAGAAAAGATCAAAGG |
| | ATCTTCTTGAGATCCTTTTTTTCTG |
| | CGCGTAATCTGCTGCTTGCAAACAA |
| | AAAAACCACCGCTACCAGCGGTGGT |
| | TTGTTTGCCGGATCAAGAGCTACCA |
| | ACTCTTTTTCCGAAGGTAACTGGCT |
| | TCAGCAGAGCGCAGATACCAAATAC |
| | TGTTCTTCTAGTGTAGCCGTAGTTA |
| | GGCCACCACTTCAAGAACTCTGTAG |
| | CACCGCCTACATACCTCGCTCTGCT |
| | AATCCTGTTACCAGTGGCTGCTGCC |
| | AGTGGCGATAAGTCGTGTCTTACCG |
| | GGTTGGACTCAAGACGATAGTTACC |
| | GGATAAGGCGCAGCGGTCGGGCTGA |
| | ACGGGGGGTTCGTGCACACAGCCCA |
| | GCTTGGAGCGAACGACCTACACCGA |
| | ACTGAGATACCTACAGCGTGAGCTA |
| | TGAGAAAGCGCCACGCTTCCCGAAG |
| | GGAGAAAGGCGGACAGGTATCCGGT |
| | AAGCGGCAGGGTCGGAACAGGAGAG |
| | CGCACGAGGGAGCTTCCAGGGGGAA |
| | ACGCCTGGTATCTTTATAGTCCTGT |
| | CGGGTTTCGCCACCTCTGACTTGAG |
| | CGTCGATTTTTGTGATGCTCGTCAG |
| | GGGGGCGGAGCCTATGGAAAAACGC |
| | CAGCAACGCGGCCTTTTTACGGTTC |
| | CTGGCCTTTTGCTGGCCTTTTGCTC |
| | ACATGTTCTTAATTAACCTGCAGGC |
| | GTTACATAACTTACGGTAAATGGCC |
| | CGCCTGGCTGACCGCCCAACGACCC |
| | CCGCCCATTGACGTCAATAATGACG |
| | TATGTTCCCATAGTAACGCCAATAG |
| | GGACTTTCCATTGACGTCAATGGGT |
| | GGAGTATTTACGGTAAACTGCCCAC |
| | TTGGCAGTACATCAAGTGTATCATA |
| | TGCCAAGTACGCCCCCTATTGACGT |
| | CAATGACGGTAAATGGCCCGCCTGG |
| | CATTATGCCCAGTACATGACCTTAT |
| | GGGACTTTCCTACTTGGCAGTACAT |
| | CTACGTATTAGTCATCGCTATTACC |
| | ATGATGATGCGGTTTTGGCAGTACA |
| | TCAATGGGCGTGGATAGCGGTTTGA |
| | CTCACGGGGATTTCCAAGTCTCCAC |
| | CCCATTGACGTCAATGGGAGTTTGT |
| | TTTGACTAGTGGAGCCGAGAGTAAT |
| | TCATACAAAAGGAGGGATCGCCTTC |
| | GCAAGGGGAGAGCCCAGGGACCGTC |
| | CCTAAATTCTCACAGACCCAAATCC |
| | CTGTAGCCGCCCCACGACAGCGCGA |
| | GGAGCATGCGCCCAGGGCTGAGCGC |
| | GGGTAGATCAGAGCACACAAGCTCA |
| | CAGTCCCCGGCGGTGGGGGGAGGGG |
| | CGCGCTGAGCGGGGGCCAGGGAGCT |
| | GGCGCGGGGCAAACTGGGAAAGTGG |
| | TGTCGTGTGCTGGCTCCGCCCTCTT |
| | CCCGAGGGTGGGGGAGAACGGTATA |
| | TAAGTGCGGTAGTCGCCTTGGACGT |
| | TCTTTTTCGCAACGGGTTTGCCGTC |
| | AGAACGCAGGTGAGTGGCGGGTGTG |
| | GCTTCCGCGGGCCCCGGAGCTGGAG |
| | CCCTGCTCTGAGCGGGCCGGGCTGA |
| | TATGCGAGTGTCGTCCGCAGGGTTT |
| | AGCTGTGAGCATTCCCACTTCGAGT |
| | GGCGGGCGGTGCGGGGGTGAGAGTG |
| | CGAGGCCTAGCGGCAACCCCGTAGC |
| | CTCGCCTCGTGTCCGGCTTGAGGCC |
| | TAGCGTGGTGTCCGCCGCCGCGTGC |
| | CACTCCGGCCGCACTATGCGTTTTT |
| | TGTCCTTGCTGCCCTCGATTGCCTT |
| | CCAGCAGCATGGGCTAACAAAGGGA |
| | GGGTGTGGGGCTCACTCTTAAGGAG |
| | CCCATGAAGCTTACGTTGGATAGGA |
| | ATGGAAGGGCAGGAGGGGCGACTGG |
| | GGCCCGCCCGCCTTCGGAGCACATG |
| | TCCGACGCCACCTGGATGGGGCGAG |
| | GCCTGTGGCTTTCCGAAGCAATCGG |
| | GCGTGAGTTTAGCCTACCTGGGCCA |
| | TGTGGCCCTAGCACTGGGCACGGTC |
| | TGGCCTGGCGGTGCCGCGTTCCCTT |
| | GCCTCCCAACAAGGGTGAGGCCGTC |
| | CCGCCCGGCACCAGTTGCTTGCGCG |
| | GAAAGATGGCCGCTCCCGGGGCCCT |
| | GTTGCAAGGAGCTCAAAATGGAGGA |
| | CGCGGCAGCCCGGTGGAGCGGGCGG |
| | GTGAGTCACCCACACAAAGGAAGAG |
| | GGCCTTGCCCCTCGCCGGCCGCTGC |
| | TTCCTGTGACCCCGTGGTCTATCGG |
| | CCGCATAGTCACCTCGGGCTTCTCT |
| | TGAGCACCGCTCGTCGCGGCGGGGG |
| | GAGGGGATCTAATGGCGTTGGAGTT |
| | TGTTCACATTTGGTGGGTGGAGACT |
| | AGTCAGGCCAGCCTGGCGCTGGAAG |
| | TCATTCTTGGAATTTGCCCCTTTGA |
| | GTTTGGAGCGAGGCTAATTCTCAAG |
| | CCTCTTAGCGGTTCAAAGGTATTTT |
| | CTAAACCCGTTTCCAGGTGTTGTGA |
| | AAGCCACCGCTAATTCAAAGCAATC |
| | CGGAGTATACATGGTTAGATCTGAC |
| | AAAACTCACACATGCCCACCGTGCC |
| | CAGCACCTGAACTCCTGGGGGGACC |
| | GTCAGTCTTCCTCTTCCCCCCAAAA |
| | CCCAAGGACACCCTCATGATCTCCC |
| | GGACCCCTGAGGTCACATGCGTGGT |
| | GGTGGACGTGGAACACGAAGACCCT |
| | GAGGTCAAGTTCAACTGGTACGTGG |
| | ACGGCGTGGAGGTGCATAATGCCAA |
| | GACAAAGCCGCGGGAGGAGCAGTAC |
| | AACAGCACGTACCGTGTGGTCAGCG |
| | TCCTCACCGTCCTGCACCAGGACTG |
| | GCTGAATGGCAAGGAGTACAAGTGC |
| | AAGGTCTCCAACAAAGCCTTCCCAG |
| | CCCCCATCGAGAAAACCATCTCCAA |
| | AGCCAAAGGGCAGCCCCGAGAACCA |
| | CAGGTGTACACCCTGCCCCCATCCC |
| | GGGAGGAGATGACCAAGAACCAGGT |
| | CAGCCTGACCTGCCTGGTCAAAGGC |
| | TTCTATCCCAGCGACATCGCCGTGG |
| | AGTGGGAGAGCAATGGGCAGCCGGA |
| | GAACAACTACAAGACCACGCCTCCC |
| | GTGCTGGACTCCGACGGCTCCTTCT |
| | TCCTCTACAGCAAGCTCACCGTGGA |
| | CAAGAGCAGGTGGCAGCAGGGGAAC |
| | GTCTTCTCATGCTCCGTGATGCACG |
| | AGGCTCTGCACAACCACTACACGCA |
| | GAAGAGCCTCTCCCTGTCTCCGGGT |
| | AAATGACCTAGGAGCAGGTTTCCCC |
| | AATGACACAAAACGTGCAACTTGAA |
| | ACTCCGCCTGGTCTTTCCAGGTCTA |
| | GAGGGGTAACACTTTGTACTGCGTT |
| | TGGCTCCACGCTCGATCCACTGGCG |
| | AGTGTTAGTAACAGCACTGTTGCTT |
| | CGTAGCGGAGCATGACGGCCGTGGG |
| | AACTCCTCCTTGGTAACAAGGACCC |
| | ACGGGGCCAAAAGCCACGCCCACAC |
| | GGGCCCGTCATGTGTGCAACCCCAG |
| | CACGGCGACTTTACTGCGAAACCCA |
| | CTTTAAAGTGACATTGAAACTGGTA |
| | CCCACACACTGGTGACAGGCTAAGG |
| | ATGCCCTTCAGGTACCCCGAGGTAA |
| | CACGCGACACTCGGGATCTGAGAAG |
| | GGGACTGGGGCTTCTATAAAAGCGC |
| | TCGGTTTAAAAAGCTTCTATGCCTG |
| | AATAGGTGACCGGAGGTCGGCACCT |
| | TTCCTTTGCAATTACTGACCCTATG |
| | AATACACTGACTGTTTGACAATTAA |
| | TCATCGGCATAGTATATCGGCATAG |
| | TATAATACGACTCACTATAGGAGGG |
| | CCACCATGATTGAACAAGATGGATT |
| | GCACGCAGGTTCTCCGGCCGCTTGG |
| | GTGGAGAGGCTATTCGGCTATGACT |
| | GGGCACAACAGACAATCGGCTGCTC |
| | TGATGCCGCCGTGTTCCGGCTGTCA |
| | GCGCAGGGGCGCCCGGTTCTTTTTG |
| | TCAAGACCGACCTGTCCGGTGCCCT |
| | GAATGAACTGCAAGACGAGGCAGCG |
| | CGGCTATCGTGGCTGGCCACGACGG |
| | GCGTTCCTTGCGCAGCTGTGCTCGA |
| | CGTTGTCACTGAAGCGGGAAGGGAC |
| | TGGCTGCTATTGGGCGAAGTGCCGG |
| | GGCAGGATCTCCTGTCATCTCACCT |
| | TGCTCCTGCCGAGAAAGTATCCATC |
| | ATGGCTGATGCAATGCGGCGGCTGC |
| | ATACGCTTGATCCGGCTACCTGCCC |
| | ATTCGACCACCAAGCGAAACATCGC |
| | ATCGAGCGAGCACGTACTCGGATGG |
| | AAGCCGGTCTTGTCGATCAGGATGA |
| | TCTGGACGAAGAGCATCAGGGGCTC |
| | GCGCCAGCCGAACTGTTCGCCAGGC |
| | TCAAGGCGAGCATGCCCGACGGCGA |
| | GGATCTCGTCGTGACACATGGCGAT |
| | GCCTGCTTGCCGAATATCATGGTGG |
| | AAAATGGCCGCTTTTCTGGATTCAT |
| | CGACTGTGGCCGGCTGGGTGTGGCG |
| | GACCGCTATCAGGACATAGCGTTGG |
| | CTACCCGTGATATTGCTGAAGAGCT |
| | TGGCGGCGAATGGGCTGACCGCTTC |
| | CTCGTGCTTTACGGTATCGCCGCTC |
| | CCGATTCGCAGCGCATCGCCTTCTA |
| | TCGCCTTCTTGACGAGTTCTTCTGA |
| | GCGGGACTCTGGGGTTCGAAATGAC |
| | CGACCAAGCGAATTCGCTAGGATTA |
| | TCCCTAATACCTGCCACCCCACTCT |
| | TAATCAGTGGTGGAAGAACGGTCTC |
| | AGAACTGTTTGTTTCAATTGGCCAT |
| | TTAAGTTTAGTAGTAAAAGACTGGT |
| | TAATGATAACAATGCATCGTAAAAC |
| | CTTCAGAAGGAAAGGAGAATGTTTT |
| | GTGGACCACTTTGGTTTTCTTTTTT |
| | GCGTGTGGCAGTTTTAAGTTATTAG |
| | TTTTTAAAATCAGTACTTTTTAATG |
| | GAAACAACTTGACCAAAAATTTGTC |
| | ACAGAATTTTGAGACCCATTAAAAA |
| | AGTTAAATGAGAAACCTGTGTGTTC |
| | CTTTGGTCAACACCGAGACATTTAG |
| | GTGAAAGACATCTAATTCTGGTTTT |
| | ACGAATCTGGAAACTTCTTGAAAAT |
| | GTAATTCTTGAGTTAACACTTCTGG |
| | GTGGAGAATAGGGTTGTTTTCCCCC |
| | CACATAATTGGAAGGGGAAGGAATA |
| | TCATTTAAAGCTATGGGAGGGTTGC |
| | TTTGATTACAACACTGGAGAGAAAT |
| | GCAGCATGTTGCTGATTGCCTGTCA |
| | CTAAAACAGGCCAAAAACTGAGTCC |
| | TTGGGTTGCATAGAAAGCTG |
| SEQ ID NO: 25 | MVRSDKTHTCPPCPAPELLGGPSVF |
| | LEPPKPKDTLMISRTPEVTCVVVDV |
| | EHEDPEVKFNWYVDGVEVHNAKTKP |
| | REEQYNSTYRVVSVLTVLHQDWLNG |
| | KEYKCKVSNKAFPAPIEKTISKAKG |
| | QPREPQVYTLPPSREEMTKNQVSLT |
| | CLVKGFYPSDIAVEWESNGQPENNY |
| | KTTPPVLDSDGSFFLYSKLTVDKSR |
| | WQQGNVFSCSVMHEALHNHYTQKSL |
| | SLSPGKGSGGGGSGGGGSGGGGSVR |
| | PVNITEPTLELLHSSCDPNAFHSTI |
| | QLYCFIYGHILNDVSVSWLMDDREI |
| | TDTLAQTVLIKEEGKLASTCSKLNI |
| | TEQQWMSESTFTCKVTSQGVDYLAH |
| | TRRCPDHEPRGVITYLIPPSPLDLY |
| | QNGAPKLTCLVVDLESEKNVNVTWN |
| | QEKKTSVSASQWYTKHHNNATTSIT |
| | SILPVVAKDWIEGYGYQCIVDHPDF |
| | PKPIVRSITKTPGQRSAPEVYVFPP |
| | PEEESEDKRTLTCLIQNFFPEDISV |
| | QWLGDGKLISNSQHSTTTPLKSNGS |
| | NQGFFIFSRLEVAKTLWTQRKQFTC |
| | QVIHEALQKPRKLEKTISTSLGNTS |
| | LRPSENLYFQGHHHHHHH |
| SEQ ID NO: 26 | MVRSDKTHTCPPCPAPELLGGPSVF |
| | LEPPKPKDTLMISRTPEVTCVVVDV |
| | EHEDPEVKFNWYVDGVEVHNAKTKP |
| | REEQYNSTYRVVSVLTVLHQDWLNG |
| | KEYKCKVSNKAFPAPIEKTISKAKG |
| | QPREPQVYTLPPSREEMTKNQVSLT |
| | CLVKGFYPSDIAVEWESNGQPENNY |
| | KTTPPVLDSDGSFFLYSKLTVDKSR |
| | WQQGNVFSCSVMHEALHNHYTQKSL |
| | SLSPGK |
| SEQ ID NO: 27 | ATGGTTAGATCTGACAAAACTCACA |
| | CATGCCCACCGTGCCCAGCACCTGA |
| | ACTCCTGGGGGGACCGTCAGTCTTC |
| | CTCTTCCCCCCAAAACCCAAGGACA |
| | CCCTCATGATCTCCCGGACCCCTGA |
| | GGTCACATGCGTGGTGGTGGACGTG |
| | AGCCACGAAGACCCTGAGGTCAAGT |
| | TCAACTGGTACGTGGACGGCGTGGA |
| | GGTGCATAATGCCAAGACAAAGCCG |
| | CGGGAGGAGCAGTACAACAGCACGT |
| | ACCGTGTGGTCAGCGTCCTCACCGT |
| | CCTGCACCAGGACTGGCTGAATGGC |
| | AAGGAGTACAAGTGCAAGGTCTCCA |
| | ACAAAGCCCTCCCAGCCCCCATCGC |
| | GAAAACCATCTCCAAAGCCAAAGGG |
| | CAGCCCCGAGAACCACAGGTGTACA |
| | CCCTGCCCCCATCCCGGGAGGAGAT |
| | GACCAAGAACCAGGTCAGCCTGACC |
| | TGCCTGGTCAAAGGCTTCTATCCCA |
| | GCGACATCGCCGTGGAGTGGGAGAG |
| | CAATGGGCAGCCGGAGAACAACTAC |
| | AAGACCACGCCTCCCGTGCTGGACT |
| | CCGACGGCTCCTTCTTCCTCTACAG |
| | CAAGCTCACCGTGGACAAGAGCAGG |
| | TGGCAGCAGGGGAACGTCTTCTCAT |
| | GCTCCGTGATGCACGAGGCTCTGCA |
| | CAACCACTACACGCAGAAGAGCCTC |
| | TCCCTGTCTCCGGGTAAAGGATCCG |
| | GCGGCGGAGGCTCCGGCGGCGGAGG |
| | CTCCGGCGGCGGAGGCTCCGTTCGA |
| | CCTGTCAACATCACTGAGCCCACCT |
| | TGGAGCTACTCCATTCATCCTGCGA |
| | CCCCAATGCATTCCACTCCACCATC |
| | CAGCTGTACTGCTTCATTTATGGCC |
| | ACATCCTAAATGATGTCTCTGTCAG |
| | CTGGCTAATGGACGATCGGGAGATA |
| | ACTGATACACTTGCACAAACTGTTC |
| | TAATCAAGGAGGAAGGCAAACTAGC |
| | CTCTACCTGCAGTAAACTCAACATC |
| | ACTGAGCAGCAATGGATGTCTGAAA |
| | GCACCTTCACCTGCAAGGTCACCTC |
| | CCAAGGCGTAGACTATTTGGCCCAC |
| | ACTCGGAGATGCCCAGATCATGAGC |
| | CACGGGGTGTGATTACCTACCTGAT |
| | CCCACCCAGCCCCCTGGACCTGTAT |
| | CAAAACGGTGCTCCCAAGCTTACCT |
| | GTCTGGTGGTGGACCTGGAAAGCGA |
| | GAAGAATGTCAATGTGACGTGGAAC |
| | CAAGAGAAGAAGACTTCAGTCTCAG |
| | CATCCCAGTGGTACACTAAGCACCA |
| | CAATAACGCCACAACTAGTATCACC |
| | TCCATCCTGCCTGTAGTTGCCAAGG |
| | ACTGGATTGAAGGCTACGGCTATCA |
| | GTGCATAGTGGACCACCCTGATTTT |
| | CCCAAGCCCATTGTGCGTTCCATCA |
| | CCAAGACCCCAGGCCAGCGCTCAGC |
| | CCCCGAGGTATATGTGTTCCCACCA |
| | CCAGAGGAGGAGAGCGAGGACAAAC |
| | GCACACTCACCTGTTTGATCCAGAA |
| | CTTCTTCCCTGAGGATATCTCTGTG |
| | CAGTGGCTGGGGGATGGCAAACTGA |
| | TCTCAAACAGCCAGCACAGTACCAC |
| | AACACCCCTGAAATCCAATGGCTCC |
| | AATCAAGGCTTCTTCATCTTCAGTC |
| | GCCTAGAGGTCGCCAAGACACTCTG |
| | GACACAGAGAAAACAGTTCACCTGC |
| | CAAGTGATCCATGAGGCACTTCAGA |
| | AACCCAGGAAACTGGAGAAAACAAT |
| | ATCCACAAGCCTTGGTAACACCTCC |
| | CTCCGTCCCTCCTAG |
| SEQ ID NO: 28 | MVRSDKTHTCPPCPAPELLGGPSVF |
| (compound B) | LFPPKPKDTLMISRTPEVTCVVVDV |
| | SHEDPEVKFNWYVDGVEVHNAKTKP |
| | REEQYNSTYRVVSVLTVLHQDWLNG |
| | KEYKCKVSNKALPAPIAKTISKAKG |
| | QPREPQVYTLPPSREEMTKNQVSLT |
| | CLVKGFYPSDIAVEWESNGQPENNY |
| | KTTPPVLDSDGSFFLYSKLTVDKSR |
| | WQQGNVFSCSVMHEALHNHYTQKSL |
| | SLSPGKGSGGGGSGGGGSGGGGSVR |
| | PVNITEPTLELLHSSCDPNAFHSTI |
| | QLYCFIYGHILNDVSVSWLMDDREI |
| | TDTLAQTVLIKEEGKLASTCSKLNI |
| | TEQQWMSESTFTCKVTSQGVDYLAH |
| | TRRCPDHEPRGVITYLIPPSPLDLY |
| | QNGAPKLTCLVVDLESEKNVNVTWN |
| | QEKKTSVSASQWYTKEIHNNATTSI |
| | TSILPVVAKDWIEGYGYQCIVDHPD |
| | FPKPIVRSITKTPGQRSAPEVYVFP |
| | PPEEESEDKRTLTCLIQNFFPEDIS |
| | VQWLGDGKLISNSQHSTTTPLKSNG |
| | SNQGFFIFSRLEVAKTLWTQRKQFT |
| | CQVIHEALQKPRKLEKTISTSLGNT |
| | SLRPS |
| SEQ ID NO: 29 | VRPVNITEPTLELLHSSCDPNAFHS |
| | TIQLYCFIYGHILNDVSVSWLMDDR |
| | EITDTLAQTVLIKEEGKLASTCSKL |
| | NITEQQWMSESTFTCKVTSQGVDYL |
| | AHTRRCPDHEPRGVITYLIPPSPLD |
| | LYQNGAPKLTCLVVDLESEKNVNVT |
| | WNQEKKTSVSASQWYTKHHNNATTS |
| | ITSILPVVAKDWIEGYGYQCIVDHP |
| | DFPKPIVRSITKTPGQRSAPEVYVE |
| | PPPEEESEDKRTLTCLIQNFFPEDI |
| | SVQWLGDGKLISNSQHSTTTPLKSN |
| | GSNQGFFIFSRLEVAKTLWTQRKQF |
| | TCQVIHEALQKPRKLEKTISTSLGN |
| | TSLRPSENLYFQGHHHEIHHH |

The chimeric binding agents of the present disclosure may comprise an IgE region with at least 70% sequence identity to an amino acid sequence set forth in any one of SEQ ID NO: 3-SEQ ID NO: 6, or SEQ ID NO: 29. The chimeric binding agents of the present disclosure may comprise an IgE region with at least 71% at least 72% at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity or sequence identity to an amino acid sequence set forth in any one of SEQ ID NO: 3-SEQ ID NO: 6, or SEQ ID NO: 29. In some instances, such chimeric binding agents can comprise two, three, or more IgE regions, wherein such two, three, or more IgE regions can be directly or indirectly, covalently of non-covalently coupled to one another, and/or can be coupled to one or more IgG region(s), thereby forming a multivalent IgE-Fc-IgG-Fc binding agent.

The chimeric binding agents of the present disclosure may comprise an IgG region with at least 70% sequence identity or sequence identity to an amino acid sequence set forth in any one of SEQ ID NO: 8-SEQ ID NO: 10, or SEQ ID NO: 26. The chimeric binding agents of the present disclosure may comprise an IgG region with at least 71% at least 72% at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to an amino acid sequence set forth in any one of SEQ ID NO: 8-SEQ ID NO: 10, or SEQ ID NO: 26. In some instances, such chimeric binding agents can comprise two, three, or more IgG regions, wherein such two, three, or more IgG regions can be directly or indirectly, covalently of non-covalently coupled to one another, and/or can be coupled to one or more IgE region(s), thereby forming a multivalent IgE-Fc-IgG-Fc binding agent.

A chimeric binding agent as described herein may comprise an IgE region with at least 70% sequence identity to an amino acid sequence set forth in any one of SEQ ID NO: 3-SEQ ID NO: 6, and an IgG region with at least 70% sequence identity to an amino acid sequence set forth in any one of SEQ ID NO: 8-SEQ ID NO: 10. A chimeric binding agent as described herein may comprise an IgE region with at least 75% sequence identity to an amino acid sequence set forth in any one of SEQ ID NO: 3-SEQ ID NO: 6, and an IgG region with at least 75% sequence identity to an amino acid sequence set forth in any one of SEQ ID NO: 8-SEQ ID NO: 10. A chimeric binding agent as described herein may comprise an IgE region with at least 80% sequence identity to an amino acid sequence set forth in any one of SEQ ID NO: 3-SEQ ID NO: 6, and an IgG region with at least 80% sequence identity to an amino acid sequence set forth in any one of SEQ ID NO: 8-SEQ ID NO: 10. A chimeric binding agent as described herein may comprise an IgE region with at least 85% sequence identity to an amino acid sequence set forth in any one of SEQ ID NO: 3-SEQ ID NO: 6, and an IgG region with at least 85% sequence identity to an amino acid sequence set forth in any one of SEQ ID NO: 8-SEQ ID NO: 10. A chimeric binding agent as described herein may comprise an IgE region with at least 90% sequence identity to an amino acid sequence set forth in any one of SEQ ID NO: 3-SEQ ID NO: 6, and an IgG region with at least 90% sequence identity to an amino acid sequence set forth in any one of SEQ ID NO: 8-SEQ ID NO: 10. A chimeric binding agent as described herein may comprise an IgE region with at least 95% sequence identity to an amino acid sequence set forth in any one of SEQ ID NO: 3-SEQ ID NO: 6, and an IgG region with at least 95% sequence identity to an amino acid sequence set forth in any one of SEQ ID NO: 8-SEQ ID NO: 10. A chimeric binding agent as described herein may comprise an IgE region with 100% sequence identity to an amino acid sequence set forth in any one of SEQ ID NO: 3-SEQ ID NO: 6, and an IgG region with 100% sequence identity to an amino acid sequence set forth in any one of SEQ ID NO: 8-SEQ ID NO: 10.

A chimeric binding agent as described herein may comprise an amino acid sequence set forth in any one of SEQ ID NO: 3-SEQ ID NO: 6 and SEQ ID NO: 8-SEQ ID NO: 10. A chimeric binding agent as disclosed herein may be a fragment comprising a contiguous fragment of any one of SEQ ID NO: 3-SEQ ID NO: 6 and SEQ ID NO: 8-SEQ ID NO: 10 that is at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36 at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 85, at least 100, at least 125, at least 150, at least 175, or at least 200 residues long, wherein the protein fragment is selected from any portion of the protein. The protein sequence may be flanked by additional amino acids. One or more additional amino acids may confer a particular in vivo charge, isoelectric point, chemical conjugation site, stability, or physiologic property to a chimeric binding agent.

A chimeric binding agent of the present disclosure may be between about 50 and about 1000 amino acid residues long. A chimeric binding agent of the present disclosure may be between about 100 and about 800 amino acid residues long. A chimeric binding agent of the present disclosure may be between about 200 and about 600 amino acid residues long. A chimeric binding agent of the present disclosure may be between about 300 and about 600 amino acid residues long. A chimeric binding agent of the present disclosure may be between about 400 and about 500 amino acid residues long.

A chimeric binding agent of the present disclosure may be at least 50 amino acid residues long. A chimeric binding agent of the present disclosure may be at least 100 amino acid residues long. A chimeric binding agent of the present disclosure may be at least 200 amino acid residues long. A chimeric binding agent of the present disclosure may be at least 250 amino acid residues long. A chimeric binding agent of the present disclosure may be at least 350 amino acid residues long. A chimeric binding agent of the present disclosure may be at least 400 amino acid residues long. A chimeric binding agent of the present disclosure may be at least 450 amino acid residues long. A chimeric binding agent of the present disclosure may be at least 500 amino acid residues long. A chimeric binding agent of the present disclosure may be at least 600 amino acid residues long. A chimeric binding agent of the present disclosure may be at least 700 amino acid residues long.

Chimeric binding agents of the present disclosure may be conjugated to, linked to, or fused to a carrier or a molecule (e.g., a small molecule, a peptide, a polypeptide, or a protein) with targeting or homing function for a cell of interest or a target cell (e.g., an immune cell). Fusion proteins or chimeric binding agents of the present disclosure may be conjugated to, linked to, or fused to a molecule that extends half-life or modifies the pharmacodynamic and/or pharmacokinetic properties of the proteins, or any combination thereof.

A chimeric binding agent may comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 positively charged residues, such as Arg or Lys, or any combination thereof. One or more lysine residues in the protein may be replaced with arginine residues. The chimeric binding agents of the present disclosure may further comprise neutral amino acid residues. The chimeric binding agent may have 75 or fewer neutral amino acid residues. The chimeric binding agent may have 81 or fewer neutral amino acid residues, 100 or fewer neutral amino acid residues, 150 or fewer neutral amino acid residues, 175 or fewer neutral amino acid residues, 200 or fewer neutral amino acid residues, 250 or fewer neutral amino acid residues, 300 or fewer neutral amino acid residues, 350 or fewer neutral amino acid residues, 400 or fewer neutral amino acid residues, or 450 or fewer neutral amino acid residues.

The chimeric binding agents of the present disclosure may further comprise negative amino acid residues. The chimeric binding agent may have at least 25 or more negative amino acid residues, 50 or more negative amino acid residues, 75 or more negative amino acid residues, 100 or more negative amino acid residues, 150 or more negative amino acid residues, or 100 or fewer negative amino acid residues. While negative amino acid residues may be selected from any negatively charged amino acid residues, the negative amino acid residues may be either E, or D, or a combination of both E and D.

The chimeric binding agents of the present disclosure may comprise a sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity or sequence identity with any one of the exemplary amino acid sequences listed in TABLE 1 (e.g., SEQ ID NO: 3-SEQ ID NO: 6 and SEQ ID NO: 8-SEQ ID NO: 10).

Two or more chimeric binding agents as disclosed herein may share a degree of sequence identity and share similar properties in vivo. A chimeric binding agent may share a degree of sequence identity with any one of the proteins of SEQ ID NO: 3-SEQ ID NO: 6 and SEQ ID NO: 8-SEQ ID NO: 10. One or more chimeric binding agents of the present disclosure may have up to about 20% pairwise sequence identity, up to about 25% pairwise sequence identity, up to about 30% pairwise sequence identity, up to about 35% pairwise sequence identity, up to about 40% pairwise sequence identity, up to about 45% pairwise sequence identity, up to about 50% pairwise sequence identity, up to about 55% pairwise sequence identity, up to about 60% pairwise sequence identity, up to about 65% pairwise sequence identity, up to about 70% pairwise sequence identity, up to about 75% pairwise sequence identity, up to about 80% pairwise sequence identity, up to about 85% pairwise sequence identity, up to about 90% pairwise sequence identity, up to about 95% pairwise sequence identity, up to about 96% pairwise sequence identity, up to about 97% pairwise sequence identity, up to about 98% pairwise sequence identity, up to about 99% pairwise sequence identity, up to about 99.5% pairwise sequence identity, or up to about 99.9% pairwise sequence identity. One or more proteins of the disclosure may have at least about 20% pairwise sequence identity, at least about 25% pairwise sequence identity, at least about 30% pairwise sequence identity, at least about 35% pairwise sequence identity, at least about 40% pairwise sequence identity, at least about 45% pairwise sequence identity, at least about 50% pairwise sequence identity, at least about 55% pairwise sequence identity, at least about 60% pairwise sequence identity, at least about 65% pairwise sequence identity, at least about 70% pairwise sequence identity, at least about 75% pairwise sequence identity, at least about 80% pairwise sequence identity, at least about 85% pairwise sequence identity, at least about 90% pairwise sequence identity, at least about 95% pairwise sequence identity, at least about 96% pairwise sequence identity, at least about 97% pairwise sequence identity, at least about 98% pairwise sequence identity, at least about 99% pairwise sequence identity, at least about 99.5% pairwise sequence identity, at least about 99.9% pairwise sequence identity with a second chimeric binding agent.

Provided herein are chimeric binding agents that comprise, consist essentially of, or consist of an amino acid sequence having at least 80%, 85%, 90%, 95%, 97%, 99%, or 100% sequence identity to the amino acid sequences set forth in SEQ ID NOs: 12, 21, 23, 25, or 28.

A chimeric binding agent as described herein may exhibit an improved Fc receptor binding affinity compared to endogenous ligands for the Fc receptor (e.g., an IgE antibody for an FcεR). A chimeric binding agent as described herein may bind one Fc receptor (e.g., a FcεR or a FcγR) with higher binding affinity compared to an endogenous ligand and bind a second Fc receptor with a comparable affinity to an endogenous ligand. A chimeric binding agent as described herein may bind two different Fc receptors (e.g., a FcεR and a FcγR) with higher affinity compared to their respective endogenous ligands.

The K_{A} and/or K_{D} values of a chimeric binding agent as described herein may be modulated and/or optimized (e.g., via amino acid substitutions) to provide improved binding affinities and binding profiles (e.g., at various pH) for one or more Fc receptors (e.g., FcεR and FcγR). A chimeric binding agent as disclosed herein may bind to an FcεR with an association constant of about 10⁵ M⁻¹ to about 10¹⁵M⁻¹. A chimeric binding agent may bind to an FcεR with an association constant of about 10⁷ M⁻¹ to about 10¹³ M⁻¹. A chimeric binding agent may bind to an FcεR with an association constant of about 10⁸ M⁻¹ to about 10¹² M⁻¹. A chimeric binding agent may bind to an FcεR with an association constant of about 10⁹ M⁻¹ to about 10¹¹M⁻¹. A chimeric binding agent may bind to an FcεR with an association constant of at least 10⁵ M⁻¹. A chimeric binding agent may bind to an FcεR with an association constant of at least 10⁷ M⁻¹. A chimeric binding agent may bind to an FcεR with an association constant of at least 10⁸ M⁻¹. A chimeric binding agent may bind to an FcεR with an association constant of at least 10¹⁰ M⁻¹. A chimeric binding agent may bind to an FcεR with an association constant of at least 10¹² M⁻¹. A chimeric binding agent may bind to an FcεR with an association constant of at least 10¹⁵ M⁻¹.

A chimeric binding agent as disclosed herein may bind to an FcγR (e.g., an FcγRIIB) with an association constant of about 10⁵ M⁻¹ to about 10¹⁵ M⁻¹. A chimeric binding agent may bind to an FcγR with an association constant of about 10⁷ M⁻¹ to about 10¹³ M⁻¹. A chimeric binding agent may bind to an FcγR with an association constant of about 10⁸ M⁻¹ to about 10¹² M⁻¹. A chimeric binding agent may bind to an FcγR with an association constant of about 10⁹ M⁻¹ to about 10¹¹ M⁻¹. A chimeric binding agent may bind to an FcγR with an association constant of at least 10⁵M⁻¹. A chimeric binding agent may bind to an FcγR with an association constant of at least 10⁷M⁻¹. A chimeric binding agent may bind to an FcγR with an association constant of at least 10⁸M⁻¹. A chimeric binding agent may bind to an FcγR with an association constant of at least 10¹⁰ M⁻¹. A chimeric binding agent may bind to an FcγR with an association constant of at least 10¹² M⁻¹. A chimeric binding agent may bind to an FcγR with an association constant of at least 10¹⁵ M⁻¹.

One or more amino acid variations in one or more functional domains of a chimeric binding agent may alter its binding affinity to one or more receptors (e.g., FcεR and FcγR) and/or its selectivity for binding one or more receptors over others. The selectivity of a chimeric binding agent for FcεRI over another FcεRs may be increased compared to an endogenous FcεR ligand (e.g., endogenous IgE molecules), and/or the selectivity for FcγRIIB over other FcγR subtypes such as FcγRIIA and/or FcγRIIB1.

The chimeric binding agents as described herein may exhibit an increases binding affinity and/or selectivity for one or more Fc receptors compared to endogenous and/or other non-naturally occurring ligands for the Fc receptors. One or more amino acid variations in one or more functional domains of a chimeric binding agent as disclosed herein may improve the binding affinity to one or more Fc receptors. The binding affinity to one or more Fc receptors (e.g., FcεRI and/or FcγRIIB) may be increased at least 10-fold, at least 20-fold, at least 50-fold, at least 100-fold, at least 150-fold, at least 200-fold, at least 500-fold, or at least 1000-fold compared to endogenous and/or other non-naturally occurring ligands for the Fc receptors.

The one or more amino acid variations in the one or more functional domains of a chimeric binding agent may improve the selectivity of the chimeric protein for one or more Fc receptors over other receptor subtypes. The selectivity for one or more Fc receptors (e.g., FcεRI and/or FcγRIIB) may be increased at least 10-fold, at least 20-fold, at least 50-fold, at least 100-fold, at least 150-fold, at least 200-fold, at least 500-fold, or at least 1000-fold compared to endogenous and/or other non-naturally occurring ligands for the Fc receptors.

A chimeric binding agent may comprise a first region comprising one or more functional domains, wherein the one or more functional domains of the first region have at least 70%, at least 75%, at least 80%, at least 90%, at least 95%, at least 99%, or 100% sequence identity to an IgE molecule of a human or a rodent. A chimeric binding agent may comprise a second region comprising one or more functional domains, wherein the one or more functional domains of the second region have at least 70%, at least 75%, at least 80%, at least 90%, at least 95%, at least 99%, or 100% sequence identity to an IgG molecule of a human or a rodent.

A chimeric binding agent may comprise one or more amino acid sequences as set forth in any one of SEQ ID NO: 3-SEQ ID NO: 6 and SEQ ID NO: 8-SEQ ID NO: 10, or a functional fragment thereof. The chimeric binding agents of the disclosure may comprise a first region having at least 70%, 80%, 90%, 95%, 99%, or 100% sequence identity or sequence identity to at least one functional domain of an IgE molecule. The chimeric binding agents of the disclosure may comprise a first region having at least 70%, 80%, 90%, 95%, 99%, or 100% sequence identity or sequence identity to at least one functional domain of an IgG molecule. The chimeric binding agents of the disclosure may further comprise a protein with 70%, 80%, 90%, 95%, 99%, or 100% sequence identity or sequence identity to any one of SEQ ID NO: 3-SEQ ID NO: 6 and SEQ ID NO: 8-SEQ ID NO: 10 or fragment thereof.

A chimeric binding agent may comprise at most 1 amino acid mutation, at most 2 amino acid mutations, at most 3 amino acid mutations, at most 4 amino acid mutations, at most 5 amino acid mutations, at most 6 amino acid mutations, at most 7 amino acid mutations, at most 8 amino acid mutations, at most 9 amino acid mutations, at most 10 amino acid mutations, or another suitable number as compared to the sequence of the venom or toxin component that the protein is derived from. A chimeric binding agent may comprise at least 1 amino acid mutation, at least 2 amino acid mutations, at least 3 amino acid mutations, at least 4 amino acid mutations, at least 5 amino acid mutations, at least 6 amino acid mutations, at least 7 amino acid mutations, at least 8 amino acid mutations, at least 9 amino acid mutations, at least 10 amino acid mutations, at least 20 amino acid mutations, at least 30 amino acid mutations, at least 50 amino acid mutations, or at least 100 amino acid mutations, or another suitable number as compared to the sequence of the immunoglobulin molecule that the protein is derived from.

A chimeric binding agent may comprise one or more regions derived from one or more functional domains of one or more different Ig molecules (e.g., IgE, IgG, or IgM). The at least one functional domain (e.g., Cε1, Cε2, Cε3, Cε4, Cγ1, Cγ2, Cγ3, or a hinge region) may comprise at least 1 amino acid variation, at least 2 amino acid variations, at 3 three amino acid variations, at least 4 amino acid variations, at least 5 amino acid variations, at least 10 amino acid variations, at least 20 amino acid variations, at least 50 amino acid variations, or at least 100 amino acid variations compared to an endogenous function domain.

The one or more amino acid variation may be located in any one or more of the functional domains Cε1, Cε2, Cε3, Cε4, Cγ1, Cγ2, Cγ3, or a hinge region that a chimeric binding agent may be comprised of.

The one or more amino acid variation may be located in a Cγ2 domain that a chimeric binding agent is comprised of. The one or more amino acid variation may be a L328F variation. The one or more amino acid variation may be a S267E variation and/or a L328F variation.

The one or more amino acid variation may be located in a Cε3 domain that a chimeric binding agent as described herein is comprised of. The one or more amino acid variation may be a T396F variation. The one or more amino acid variation may be located in a Cε4 domain that a chimeric binding agent as described herein is comprised of A chimeric binding agent as disclosed herein may comprise a S267E variation and a L328F variation in its Cγ2 domain, a T396F variation in its Cε3 domain, and one or more amino acid variations in its Cε4 domain, or any combination thereof.

Amino acid variations may be introduced into a protein (e.g., a chimeric binding agent) as disclosed herein to provide a protein that has one or more specific physicochemical properties such as a specific charge, stability, and/or solubility at a certain pH such as a physiological pH.

The present disclosure also encompasses multimers of the various chimeric binding agents described herein. Examples of multimers include dimers, trimers, tetramers, pentamers, hexamers, heptamers, and so on. A multimer may be a homomer formed from a plurality of identical subunits or a heteromer formed from a plurality of different subunits. A chimeric binding agent of the present disclosure may be arranged in a multimeric structure with at least one other protein, or two, three, four, five, six, seven, eight, nine, ten, or more other proteins. The chimeric binding agents of a multimeric structure each may have the same amino acid sequence or a different amino acid sequence.

A chimeric binding agent of the present disclosure may comprise at least one, at least two, at least three, at least four, at least five, at least 6, at least seven, at least eight, at least nine, or at least ten functional domains from or derived from one or more different Ig molecules (e.g., IgE and IgG1). The functional domains that a chimeric binding agent may be comprised of may be selected from Cε1, Cε2, Cε3, Cε4, Cγ1, Cγ2, Cγ3, or a hinge region. A chimeric binding agent of the present disclosure (e.g., an IgG-Fc-IgE-Fc protein) may comprise one Cε2 domain, one Cε3 domain, one Cε4 domain, one Cγ2 domain, and one Cγ3 domain. The one or more functional domains may be connected to one another via a linker such as a flexible linker. The flexible linker may be GGGGS (SEQ ID NO: 13).

As further described herein, the chimeric binding agents of the present disclosure may be capable of binding one or more different receptors, such as Fc receptors via their Fc binding domains. The region of a chimeric binding agent that is from or derived from an IgE molecule may bind to an FCC receptor (e.g., a FcεRI). The IgE region of a chimeric binding agent may comprise the domains Cε2, Cε3, and Cε4. An IgE region comprising the Cε3 and Cε4 functional domains may bind to an FcεR (e.g., a FcεRI) with an association constant of about 10⁸ M⁻¹ to about 10¹² M⁻¹. An IgG (e.g., IgG1) region of a chimeric binding agent may comprise the domains Cγ2 and Cγ3. An IgG region comprising the Cγ2 and Cγ3 functional domains may bind to an FcγR (e.g., a FcγRIIB) with an association constant of about 107 M⁻¹ to about 10¹³ M⁻¹. A chimeric binding agent comprising an FcεR-binding region (e.g., those regions comprising the IgE domains Cε2, Cε3, and Cε4) and an FcγR-binding region (e.g., those regions comprising the IgG domains Cγ2 and Cγ3) may bind to an FcεR and an FcγR simultaneously. This binding to an FcεR and an FcγR (e.g., the simultaneous binding to an FcεR and an FcγR) may be particularly useful for the prevention and/or treatment of various diseases and conditions such as allergic diseases including IgE-mediated diseases and conditions. Without being bound to any theory, the particular use of the herein disclosed chimeric binding agents may be due to the simultaneous binding to an FcεR in an antagonistic manner (e.g., blocking the FcεR) and to an FcγR in an agonistic manner (activating the FcγR), thereby initiating, promoting, or enhancing ITIM signaling.

### Chemical Modification of Binding Agents

A chimeric binding agent as described herein may be chemically modified in one or more of a variety of ways. The chimeric binding agent may be mutated to add function (e.g., improve the binding affinity and/or selectivity for one or more receptors such as Fc receptors), delete function, or modify the in vivo behavior. Amino acids may also be varied, such as to increase half-life, modify, add or delete binding behavior in vivo, add new targeting function, modify secondary, tertiary or quaternary structure of the protein, or allow conjugation sites.

A chemical modification may extend the half-life of a protein or change the biodistribution or pharmacokinetic profile of the protein. A chemical modification may comprise the chemical conjugation of a polymer, a polyether, polyethylene glycol, a biopolymer, a polyamino acid, a fatty acid, a dendrimer, an Fc region, a simple saturated carbon chain such as palmitate or myristolate, or albumin to a protein (e.g., a chimeric binding agent) of the present disclosure. A polyamino acid may include a poly amino acid sequence with repeated single amino acids (e.g., poly glycine), and a poly amino acid sequence with mixed poly amino acid sequences (e.g., gly-ala-gly-ala) that may or may not follow a pattern, or any combination of the foregoing.

The chimeric binding agents of the present disclosure may be modified such that the modification may increase the stability and/or the half-life of the proteins. The attachment of a hydrophobic moiety, such as to the N-terminus, the C-terminus, or an internal amino acid, may be used to extend half-life of a protein of the present disclosure. The proteins as disclosed herein may also be modified to increase or decrease the gut permeability or cellular permeability of the protein. The proteins of the present disclosure may show high accumulation in the intestine, demonstrating applicability in the treatment and-or prevention of diseases or conditions of the intestines, such as Crohn's disease or more generally inflammatory bowel diseases. The proteins of the present disclosure may include post-translational modifications (e.g., methylation and/or amidation and/or glycosylation), which may affect, e.g., serum half-life. Linear and/or branched carbon chains (e.g., by myristoylation and/or palmitylation) may be conjugated to, linked to, the fusion proteins or proteins. The linear and/or branched carbon chains may render the fusion proteins or proteins easily separable from the unconjugated material. Methods that may be used to separate the fusion proteins or proteins from the unconjugated material include, but are not limited to, solvent extraction and reverse phase chromatography. Lipophilic moieties may extend half-life through reversible binding to serum albumin. Conjugated moieties can, e.g., be lipophilic moieties that extend half-life of the proteins through reversible binding to serum albumin. The lipophilic moiety may be cholesterol or a cholesterol derivative including cholestenes, cholestanes, cholestadienes and oxysterols. The proteins of the present disclosure may be conjugated to or linked to myristic acid (tetradecanoic acid) or a derivative thereof. The proteins of the present disclosure may conjugated to or linked to a half-life modifying agent. Examples of half-life modifying agents may include, but is not limited to: a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, or a molecule that binds to albumin. Additionally, conjugation of the protein to a near infrared dye, such as Cy5.5, or to an albumin binder such as Albu-tag may extend serum half-life of any protein as described herein. Immunogenicity may be reduced by using minimal non-human protein sequences to extend serum half-life of the protein.

One or more amino acid residues may be added to the N-terminus of SEQ ID NO: 3-SEQ ID NO: 6 and SEQ ID NO: 8-SEQ ID NO: 10 to serve as a spacer or linker in order to facilitate conjugation or fusion to another molecule, as well as to facilitate cleavage of the protein from such conjugated to, linked to, or fused molecules. The chimeric binding agents of the present disclosure may be conjugated to, linked to, or fused to other moieties that, e.g., may modify or effect changes to the properties of the proteins.

### Linkers

The herein described chimeric binding agents may comprise one or more linker moieties. A linker moiety may link an FcγR binding region comprising one or more FcγR binding domains to an FcεR binding region comprising one or more FcεR binding domains. A linker may also be used to link one or more Fc receptor binding domains that a chimeric binding agent of the present disclosure may be comprised of, such as linking the Cε2, Cε3, and/or Cε4 domains.

A first region (e.g., an FcεR binding region) comprising one or more Cc domains may be linked to a second region (e.g., an FcγR binding region) comprising one or more Cy domains via a glycine-rich linker. The glycine rich linker may be Gₓ wherein x=1, 2, 3, 4, 5, 6, 7, 8, 9, 10, 11, 12, 15, 20, and so forth. The glycine rich linker may be (GGGGS)ₓ (SEQ ID NO: 29), wherein x=1, 2, 3, 4, 5, 6, 7, 8, 9, 10, 11, 12, 15, 20, and so forth. The glycine-rich linker may comprise the amino acid sequence GGGGS (SEQ ID NO: 13) or GGGGSGGGGSGGGGS ((=GGGGS)₃, SEQ ID NO: 14). A diprotein such as GS may be added as the first two N-terminal amino acids of the amino acid sequence of any chimeric binding agent disclosed herein, such as those having an amino acid sequence set forth in any one of SEQ ID NO: 3-SEQ ID NO: 6 and SEQ ID NO: 8-SEQ ID NO: 10. The diprotein (e.g., GS) may be used as a linker or used to couple to a linker to form a protein conjugate or fusion molecules such as a chimeric binding agent, or fusion molecule that comprises a chimeric binding agent conjugate to, linked to, or fused to another functional molecule such as therapeutic or diagnostic agent. A linker may comprise a GₓS_{y} protein, wherein x and y independently may be any whole number, such as 1, 2, 3, 4, 5, 6, 7, 8, 9, and 10. A linker may comprise (GS)ₓ (SEQ ID NO: 19), wherein x may be any whole number, such as 1, 2, 3, 4, 5, 6, 7, 8, 9, and 10. A linker may comprise GGSSG (SEQ ID NO: 15), GGGGG (SEQ ID NO: 16), GSGSGSGS (SEQ ID NO: 17), GGGS (SEQ ID NO: 18), or a variant or fragment thereof.

A linker moiety as disclosed herein may be a cleavable, a stable, a self-immolating, a hydrophilic, or a hydrophobic linker moiety. The linkers used herein may comprise at least two functional groups (e.g., amino and carboxyl groups), one bonded to a first region of a chimeric binding agent, and one bonded to a second region of a chimeric binding agent, and a linking portion between the two functional groups.

Non-limiting examples of the linking portion may include alkylene, alkenylene, alkynylene, polyether, such as polyethylene glycol (PEG), polyester, polyamide, polyamino acids, polyproteins, cleavable proteins, Val-Cit, Phe-Lys, Val-Lys, Val-Ala, other protein linkers as given in Doronina et al., 2008, linkers cleavable by beta glucuronidase, linkers cleavable by a cathepsin or by cathepsin B, D, E, H, L, S, C, K, O, F, V, X, or W, Val-Cit-p-aminobenzyloxycarbonyl, glucuronide-MABC, aminobenzylcarbamates, D-amino acids, and polyamine, any of which being unsubstituted or substituted with any number of substituents, such as halogens, hydroxyl groups, sulfhydryl groups, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, urethane groups, epoxides, charged groups, zwitterionic groups, and ester groups. Other non-limiting examples of reactions to link, fuse, or conjugate molecules together include click chemistry, copper-free click chemistry, HIPS ligation, Staudinger ligation, and hydrazine-iso-Pictet-Spengler.

A linker of the present disclosure may comprise a cleavable or stable linker moiety. Cleavable linkers of the present disclosure may include protease cleavable protein linkers, nuclease sensitive nucleic acid linkers, lipase sensitive lipid linkers, glycosidase sensitive carbohydrate linkers, pH sensitive linkers, hypoxia sensitive linkers, photo-cleavable linkers, heat-labile linkers, enzyme cleavable linkers (e.g., esterase cleavable linker), ultrasound-sensitive linkers, and X-ray cleavable linkers. Such linker may be a non-cleavable linker.

In some instances, two or more Ig-Fc regions (e.g., IgE-Fc and/or IgG-Fc regions) can be coupled or linked to one another via one or more hinge region(s) of at least one or more Ig-Fc regions. As an example, a chimeric binding agent can comprise, from the N- to the C-terminus, an IgE-Fc region that is coupled to an IgG-Fc region via the hinge region of the IgG-Fc region, e.g., as depicted in FIG. 1D and FIG. 1F.

### Pharmacokinetics of Binding Agents

The pharmacokinetics of any of the fusion proteins or chimeric binding agents of the present disclosure may be determined after administration of the protein via different routes of administration. The pharmacokinetic parameters of a protein of this disclosure may be quantified after subcutaneous, intramuscular, rectal, aerosol, parenteral, ophthalmic, pulmonary, transdermal, vaginal, optic, nasal, oral, sublingual, inhalation, dermal, intrathecal, intranasal, peritoneal, buccal, synovial, intratumoral, intravenous, or topical administration. The pharmacokinetic and/or pharmacodynamic properties of the proteins (e.g., chimeric binding agents) of the present disclosure may be evaluated and analyzed by using tracking agents such as radiolabels or fluorophores. A radiolabeled fluorophore-labeled chimeric binding agent of this disclosure may be administered via various routes of administration, followed by determining the protein concentration or dose recovery in various biological samples such as plasma, urine, feces, any organ, skin, muscle, and other tissues may be determined using a range of methods including HPLC, fluorescence detection techniques (TECAN quantification, flow cytometry, iVIS), gamma-counting, or liquid scintillation counting.

The methods and compositions described herein may relate to pharmacokinetics of protein administration via any route to a subject. Pharmacokinetics may be described using methods and models such as compartmental models or noncompartmental methods. Compartmental models may include but are not limited to monocompartmental model, the two compartmental model, the multicompartmental model or the like. Models may be divided into different compartments and may be described by the corresponding scheme. One scheme may be the absorption, distribution, metabolism and excretion (ADME) scheme. Another scheme may be the liberation, absorption, distribution, metabolism and excretion (LADME) scheme. Metabolism and excretion may be grouped into one compartment referred to as the elimination compartment. Liberation may include liberation of an additional therapeutic or diagnostic molecules that may be conjugated to, linked to, or fused to chimeric binding agent, and absorption may include absorption of the protein by the subject, distribution includes distribution of the composition through the blood plasma and to different tissues, metabolism, which includes metabolism or inactivation of the composition and excretion, which may include excretion or elimination of the composition or the products of metabolism of the composition. Compositions administered subcutaneously to a subject may be subject to multiphasic pharmacokinetic profiles, which may include but are not limited to aspects of tissue distribution and metabolism/excretion. As such, the decrease in plasma or serum concentration of the composition is often biphasic, including an alpha phase and a beta phase, occasionally a gamma, delta or other phase is observed.

Pharmacokinetics may include determining at least one parameter associated with administration of a protein to a subject. Such parameters may include at least the dose (D), dosing interval (τ), area under curve (AUC), maximum concentration (Cₘₐₓ), minimum concentration reached before a subsequent dose is administered (Cₘᵢₙ), minimum time (Tₘᵢₙ), maximum time to reach Cₘₐₓ (Tₘₐₓ), volume of distribution (V_{d}), steady-state volume of distribution (Vₛₛ), back-extrapolated concentration at time 0 (C₀), steady state concentration (Cₛₛ), elimination rate constant (kₑ), infusion rate (kᵢₙ), clearance (CL), bioavailability (f), fluctuation (% PTF) and elimination half-life (t_{1/2}).

A chimeric binding agent comprising an amino acid sequence set forth in any one of SEQ ID NO: 3-SEQ ID NO: 6 or SEQ ID NO: 8-SEQ ID NO: 10, or a combination thereof, may exhibit optimal pharmacokinetic parameters after subcutaneous oral administration. Chimeric binding agents comprising an amino acid sequence set forth in any one of SEQ ID NO: 3-SEQ ID NO: 6 or SEQ ID NO: 8-SEQ ID NO: 10, or a combination thereof, may exhibit optimal pharmacokinetic parameters after any route of administration, such as subcutaneous, oral administration, inhalation, intranasal administration, topical administration, intravenous administration, subcutaneous administration, intra-articular administration, intramuscular administration, intraperitoneal administration, intra-synovial, or any combination thereof.

Chimeric binding agents comprising an amino acid sequence set forth in any one of SEQ ID NO: 3-SEQ ID NO: 6 or SEQ ID NO: 8-SEQ ID NO: 10, or a combination thereof, may exhibit an average Tₘₐₓ of 0.5-12 hours, or 1-48 hours, or 1-72 hours at which the Cₘₐₓ is reached, an average bioavailability in serum of 10-100% after parenteral administration, an average bioavailability in serum of 0.1%-90% in the subject after administering the protein to the subject by a subcutaneous route, an average t_{1/2} of 0.1 hours-168 hours, or 0.25 hours-48 hours in a subject after administering the protein to the subject, an average clearance (CL) of 0.5-100 L/hour or 0.5-50 L/hour of the protein after administering the protein to a subject, an average volume of distribution (V_{d}) of 200-50,000 mL in the subject after systemically administering the protein to the subject, or optionally no systemic uptake, any combination thereof.

### Sequence Identity, Homology, and Similarity

As disclosed herein, percent (%) sequence identity or homology may be determined by conventional methods. (See e.g., Altschul et al. (1986), Bull. Math. Bio. 48:603 (1986), and Henikoff and Henikoff (1992), Proc. Natl. Acad. Sci. USA 89:10915). Briefly, two amino acid sequences may be aligned to optimize the alignment scores using a gap opening penalty of 10, a gap extension penalty of 1, and the “BLOSUM62” scoring matrix of Henikoff and Henikoff (Id.). The sequence identity or homology is then calculated as: ([Total number of identical matches]/[length of the longer sequence plus the number of gaps introduced into the longer sequence in order to align the two sequences])(100).

As disclosed herein, percent (%) sequence similarity may be determined by conventional methods. (See e.g., Altschul et al. (1986), Bull. Math. Bio. 48:603 (1986), and Henikoff and Henikoff (1992), Proc. Natl. Acad. Sci. USA 89:10915). Briefly, two amino acid sequences may be aligned to optimize the alignment scores using a gap opening penalty of 10, a gap extension penalty of 1, and the “BLOSUM62” scoring matrix of Henikoff and Henikoff (Id.). The sequence similarity is then calculated as: ([Total number of identical matches]/[length of the longer sequence plus the number of gaps introduced into the longer sequence in order to align the two sequences])(100).

Additionally, there may be several established algorithms available to align two amino acid sequences. The “FASTA” similarity search algorithm of Pearson and Lipman may be a suitable protein alignment method for examining the level of sequence identity or homology shared by an amino acid sequence of a protein disclosed herein and the amino acid sequence of a protein variant. The FASTA algorithm is described, for example, by Pearson and Lipman, Proc. Nat'l Acad. Sci. USA 85:2444 (1988), and by Pearson, Meth. Enzymol. 183:63 (1990). Briefly, FASTA first characterizes sequence similarity by identifying regions shared by the query sequence (e.g., SEQ ID NO: 1) and a test sequence that has either the highest density of identities (if the ktup variable is 1) or pairs of identities (if ktup=2), without considering conservative amino acid substitutions, insertions, or deletions. The ten regions with the highest density of identities may then be rescored by comparing the similarity of all paired amino acids using an amino acid substitution matrix, and the ends of the regions may be “trimmed” to include generally those residues that contribute to the highest score. If there are several regions with scores greater than the “cutoff” value (calculated by a predetermined formula based upon the length of the sequence and the ktup value), then the trimmed initial regions may be examined to determine whether the regions may be joined to form an approximate alignment with gaps. Finally, the highest scoring regions of the two amino acid sequences may be aligned using a modification of the Needleman-Wunsch-Sellers algorithm (Needleman and Wunsch, J. Mol. Biol. 48:444 (1970); Sellers, Siam J. Appl. Math. 26:787 (1974)), which allows for amino acid insertions and deletions. Illustrative parameters for FASTA analysis are: ktup=1, gap opening penalty=10, gap extension penalty=1, and substitution matrix=BLOSUM62. These parameters may be introduced into a FASTA program by modifying the scoring matrix file (“SMATRIX”), as explained in Appendix 2 of Pearson, Meth. Enzymol. 183:63 (1990).

FASTA may also be used to determine the sequence identity or homology of nucleic acid sequences or molecules using a ratio as disclosed above. For nucleic acid sequence comparisons, the k-tup value may range between about one to about six, from about three to six, or about three, with other parameters set as described above.

Amino acids that may be a “conservative amino acid substitution” may be illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies may be used to define conservative amino acid substitutions that may be introduced into the amino acid sequences of the present disclosure. Although it is possible to design amino acid substitutions based solely upon chemical properties (as discussed above), the language “conservative amino acid substitution” generally refers to a substitution represented by a BLOSUM62 value of greater than −1. An amino acid substitution is conservative if the substitution is characterized by a BLOSUM62 value of 0, 1, 2, or 3. According to this system, conservative amino acid substitutions may be characterized by a BLOSUM62 value of at least 1 (e.g., 1, 2 or 3), while other conservative amino acid substitutions may be characterized by a BLOSUM62 value of at least 2 (e.g., 2 or 3).

Determination of amino acid residues that may be within regions or domains that may be critical to maintaining structural integrity may be determined. Within these regions one may determine specific residues that may be more or less tolerant of change and maintain the overall tertiary structure of the molecule. Methods for analyzing sequence structure include, but are not limited to, alignment of multiple sequences with high amino acid or nucleotide identity or homology and computer analysis using available software (e.g., the Insight II® viewer and homology modeling tools; MSI, San Diego, Calif.), secondary structure propensities, binary patterns, complementary packing and buried polar interactions (Barton, G. J., Current Opin. Struct. Biol. 5:372-6 (1995) and Cordes, M. H. et al., Current Opin. Struct. Biol. 6:3-10 (1996)). In general, when designing modifications to molecules or identifying specific fragments, determination of structure may typically be accompanied by evaluating activity of modified molecules.

### Chimeric Binding Agents and Conjugates Thereof

A chimeric binding agent of the present disclosure may be conjugated to, linked to, or fused to a molecule or agent used in imaging, research, therapeutics, theranostics, pharmaceuticals, chemotherapy, chelation therapy, targeted drug delivery, and radiotherapy. A protein of the present disclosure may be conjugated to, linked to, or fused with detectable agents, such as a fluorophore, a near-infrared dye, a contrast agent, a nanoparticle, a metal-containing nanoparticle, a metal chelate, an X-ray contrast agent, a PET agent, a metal, a radioisotope, a dye, radionuclide chelator, or another suitable material that may be used in imaging.

A chimeric binding agent or fusion protein as disclosed herein may be linked to 1, 2, 3, 4, 5, 6, 7, 8, 9, or 10 detectable agents. Non-limiting examples of radioisotopes that may be used as detectable agents include alpha emitters, beta emitters, positron emitters, and gamma emitters. The metal or radioisotope may be selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. The metal may be actinium, bismuth, lead, radium, strontium, samarium, or yttrium. The radioisotope may be actinium-225 or lead-212. The near-infrared dyes that may be used in combination with the herein described chimeric binding agents may not be easily quenched by biological tissues and fluids. The fluorophore may be a fluorescent agent emitting electromagnetic radiation at a wavelength between 650 nm and 4000 nm, such emissions being used to detect such agent. Non-limiting examples of fluorescent dyes that may be used as a conjugating molecule in the present disclosure include DyLight-680, DyLight-750, VivoTag-750, DyLight-800, IRDye-800, VivoTag-680, Cy5.5, or indocyanine green (ICG). Near infrared dyes may include cyanine dyes (e.g., Cy7, Cy5.5, and Cy5). Additional non-limiting examples of fluorescent dyes for use as a conjugating molecule in the present disclosure may include acradine orange or yellow, Alexa Fluors (e.g., Alexa Fluor 790, 750, 700, 680, 660, and 647) and any derivative thereof, 7-actinomycin D, 8-anilinonaphthalene-1-sulfonic acid, ATTO dye and any derivative thereof, auramine-rhodamine stain and any derivative thereof, bensantrhone, bimane, 9-10-bis(phenylethynyl)anthracene, 5,12-bis(phenylethynyl)naththacene, bisbenzimide, brainbow, calcein, carbodyfluorescein and any derivative thereof, 1-chloro-9,10-bis(phenylethynyl)anthracene and any derivative thereof, DAPI, DiOC6, DyLight Fluors and any derivative thereof, epicocconone, ethidium bromide, FlAsH-EDT2, Fluo dye and any derivative thereof, FluoProbe and any derivative thereof, Fluorescein and any derivative thereof, Fura and any derivative thereof, GelGreen and any derivative thereof, GelRed and any derivative thereof, fluorescent proteins and any derivative thereof, m isoform proteins and any derivative thereof such as mCherry, hetamethine dye and any derivative thereof, hoeschst stain, iminocoumarin, indian yellow, indo-1 and any derivative thereof, laurdan, lucifer yellow and any derivative thereof, luciferin and any derivative thereof, luciferase and any derivative thereof, mercocyanine and any derivative thereof, nile dyes and any derivative thereof, perylene, phloxine, phyco dye and any derivative thereof, propium iodide, pyranine, rhodamine and any derivative thereof, ribogreen, RoGFP, rubrene, stilbene and any derivative thereof, sulforhodamine and any derivative thereof, SYBR and any derivative thereof, synapto-pHluorin, tetraphenyl butadiene, tetrasodium tris, Texas Red, Titan Yellow, TSQ, umbelliferone, violanthrone, yellow fluroescent protein and YOYO-1. Other Suitable fluorescent dyes may include, but are not limited to, fluorescein and fluorescein dyes (e.g., fluorescein isothiocyanine or FITC, naphthofluorescein, 4′,5′-dichloro-2′,7′-dimethoxyfluorescein, 6-carboxyfluorescein or FAM, etc.), carbocyanine, merocyanine, styryl dyes, oxonol dyes, phycoerythrin, erythrosin, eosin, rhodamine dyes (e.g., carboxytetramethyl-rhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Oregon Green Dyes (e.g., Oregon Green 488, Oregon Green 500, Oregon Green 514, etc.), Texas Red, Texas Red-X, SPECTRUM RED, SPECTRUM GREEN, cyanine dyes (e.g., CY-3, Cy-5, CY-3.5, CY-5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), and the like. Additional suitable detectable agents are described in PCT/US14/56177. Non-limiting examples of radioisotopes include alpha emitters, beta emitters, positron emitters, and gamma emitters. The metal or radioisotope may be selected from the group consisting of actinium, americium, bismuth, cadmium, cesium, cobalt, europium, gadolinium, iridium, lead, lutetium, manganese, palladium, polonium, radium, ruthenium, samarium, strontium, technetium, thallium, and yttrium. The metal may be actinium, bismuth, lead, radium, strontium, samarium, or yttrium. The radioisotope may be actinium-225 or lead-212. Additionally, the following radionuclides may be used for diagnosis and/or therapy: carbon (e.g., ¹¹C or ¹⁴C), nitrogen (e.g., ¹³N), fluorine (e.g., ¹⁸F), gallium (e.g., ⁶⁷Ga or ⁶⁸Ga), copper (e.g., ⁶⁴Cu or ⁶⁷Cu), zirconium (e.g., ⁸⁹Zr), lutetium (e.g., ¹⁷⁷Lu).

The chimeric binding agents as disclosed herein may be conjugated to, linked to, or fused to a radiosensitizer or photosensitizer. Examples of radiosensitizers may include but are not limited to: ABT-263, ABT-199, WEHI-539, paclitaxel, carboplatin, cisplatin, oxaliplatin, gemcitabine, etanidazole, misonidazole, tirapazamine, and nucleic acid base derivatives (e.g., halogenated purines or pyrimidines, such as 5-fluorodeoxyuridine). Examples of photosensitizers may include but are not limited to: fluorescent molecules or beads that generate heat when illuminated, nanoparticles, porphyrins and porphyrin derivatives (e.g., chlorins, bacteriochlorins, isobacteriochlorins, phthalocyanines, and naphthalocyanines), metalloporphyrins, metallophthalocyanines, angelicins, chalcogenapyrrillium dyes, chlorophylls, coumarins, flavins and related compounds such as alloxazine and riboflavin, fullerenes, pheophorbides, pyropheophorbides, cyanines (e.g., merocyanine 540), pheophytins, sapphyrins, texaphyrins, purpurins, porphycenes, phenothiaziniums, methylene blue derivatives, naphthalimides, nile blue derivatives, quinones, perylenequinones (e.g., hypericins, hypocrellins, and cercosporins), psoralens, quinones, retinoids, rhodamines, thiophenes, verdins, xanthene dyes (e.g., eosins, erythrosins, rose bengals), dimeric and oligomeric forms of porphyrins, and prodrugs such as 5-aminolevulinic acid. Advantageously, this approach may allow for highly specific targeting of diseased cells (e.g., cancer cells) using both a therapeutic agent (e.g., drug) and electromagnetic energy (e.g., radiation or light) concurrently. The proteins of the present disclosure may be conjugated to, linked to, fused with, or covalently or non-covalently linked to the agent, e.g., directly or via a linker.

A radionuclide may be attached to a protein (e.g., a chimeric binding agent) as described herein using a chelator. Exemplary chelator moieties may include 2,2′,2″-(3-(4-(3-(1-(4-(1,2,4,5-tetrazin-3-yl)phenyl)-1-oxo-5,8,11,14,17,20,23-heptaoxa-2-azapentacosan-25-yl)thioureido)benzyl)-1,4,7-triazonane-2,5,8-triyl)triacetic acid; 2,2′,2″-(3-(4-(3-(1-(4-(1,2,4,5-tetrazin-3-yl)phenyl)-1-oxo-5,8,11,14,17,20,23,26,29,32,35-undecaoxa-2-azaheptatriacontan-37-yl)thioureido)benzyl)-1,4,7-triazonane-2,5,8-triyl)triacetic acid; 2,2′-(7-(4-(3-(1-(4-(1,2,4,5-tetrazin-3-yl)phenyl)-1-oxo-5,8,11,14,17,20,23,26,29,32,35-undecaoxa-2-azaheptatriacontan-37-yl)thioureido)benzyl)-1,4,7-triazonane-1,4-diyl)diacetic acid; 2,2′,2″-(3-(4-(3-(1-(4-(1,2,4,5-tetrazin-3-yl)phenyl)-3,7-dioxo-11,14,17,20,23,26,29-heptaoxa-2,8-diazahentriacontan-31-yl)thioureido)benzyl)-1,4,7-triazonane-2,5,8-triyl)triacetic acid; 2,2′,2″-(3-(4-(3-(1-(4-(1,2,4,5-tetrazin-3-yl)phenyl)-3,7-dioxo-11,14,17,20,23,26,29,32,35,38,41-undecaoxa-2,8-diazatritetracontan-43-yl)thioureido)benzyl)-1,4,7-triazonane-2,5,8-triyl)triacetic acid; 2,2′,2″-(3-(4-(3-(25,28-dioxo-28-((6-(6-(pyridin-2-yl)-1,2,4,5-tetrazin-3-yl)pyridin-3-yl)amino)-3,6,9,12,15,18,21-heptaoxa-24-azaoctacosyl)thioureido)benzyl)-1,4,7-triazonane-2,5,8-triyl)triacetic acid; 2,2′,2″-(3-(4-(3-(37,40-dioxo-40-((6-(6-(pyridin-2-yl)-1,2,4,5-tetrazin-3-yl)pyridin-3-yl)amino)-3,6,9,12,15,18,21,24,27,30,33-undecaoxa-36-azatetracontyl)thioureido)benzyl)-1,4,7-triazonane-2,5,8-triyl)triacetic acid; 2,2′,2″-(3-(4-(1-(4-(6-methyl-1,2,4,5-tetrazin-3-yl)phenyl)-3-oxo-6,9,12,15,18,21,24-heptaoxa-2-azaheptacosan-27-amido)benzyl)-1,4,7-triazonane-2,5,8-triyl)triacetic acid; 2,2′,2″-(2-(4-(1-(4-(6-methyl-1,2,4,5-tetrazin-3-yl)phenoxy)-3,6,9,12,15,18,21,24,27,30,33-undecaoxahexatriacontan-36-amido)benzyl)-1,4,7-triazonane-1,4,7-triyl)triacetic acid; 2,2′,2″-(3-(4-(3-(5-amino-6-((4-(6-methyl-1,2,4,5-tetrazin-3-yl)benzyl)amino)-6-oxohexyl)thioureido)benzyl)-1,4,7-triazonane-2,5,8-triyl)triacetic acid; 2,2′-(7-(4-(3-(5-amino-6-((4-6-methyl-1,2,4,5-tetrazin-3-yl)benzyl)amino)-6-oxohexyl)thioureido)benzyl)-1,4,7-triazonane-1,4-diyl)diacetic acid; 2,2′,2″-(3-(4-(3-(5-amino-6-((5-amino-6-((4-(6-methyl-1,2,4,5-tetrazin-3-yl)benzyl)amino)-6-oxohexyl)amino)-6-oxohexyl)thioureido)benzyl)-1,4,7-triazonane-2,5,8-triyl)triacetic acid; and 2,2′,2″-(3-(4-(3-(5-amino-6-((5-amino-6-((5-amino-6-((4-(6-methyl-1,2,4,5-tetrazin-3-yl)benzyl)amino)-6-oxohexyl)amino)-6-oxohexyl)amino)-6-oxohexyl)thioureido)benzyl)-1,4,7-triazonane-2,5,8-triyl)triacetic acid.

### Methods of Manufacture

Various expression vector/host systems may be utilized for the recombinant expression and production of chimeric binding agents described herein. Non-limiting examples of such systems include microorganisms such as bacteria transformed with recombinant bacteriophage DNA, plasmid DNA or cosmid DNA expression vectors containing a nucleic acid sequence encoding proteins (e.g., those having a nucleotide sequence of any one of SEQ ID NO: 1, SEQ ID NO: 2, SEQ ID NO: 7, and SEQ ID NOs: 11, 20, 22, 24, and 27), protein constructs, or protein fusion proteins/chimeric proteins described herein, yeast transformed with recombinant yeast expression vectors containing the aforementioned nucleic acid sequence, insect cell systems infected with recombinant virus expression vectors (e.g., baculovirus) containing the aforementioned nucleic acid sequence, plant cell systems infected with recombinant virus expression vectors (e.g., cauliflower mosaic virus (CaMV), tobacco mosaic virus (TMV)), or transformed with recombinant plasmid expression vectors (e.g., Ti plasmid) containing the aforementioned nucleic acid sequence, or animal cell systems infected with recombinant virus expression vectors (e.g., adenovirus, vaccinia virus, lentivirus) including cell lines engineered to contain multiple copies of the aforementioned nucleic acid sequence, either stably amplified (e.g., CHO/dhfr, CHO/glutamine synthetase) or unstably amplified in double-minute chromosomes (e.g., murine cell lines).

A host cell may be adapted to express one or more proteins described herein. The host cells may be prokaryotic, eukaryotic, or insect cells. Host cells may be capable of modulating the expression of the inserted sequences or modifying and processing the gene or protein product in the specific fashion specific. Expression from certain promoters may be elevated in the presence of certain inducers (e.g., zinc and cadmium ions for metallothionine promoters). Modifications (e.g., phosphorylation) and processing (e.g., cleavage) of protein products as described herein may be important for the function of the protein. Host cells may have characteristic and specific mechanisms for the post-translational processing and modification of a protein. The host cells used to express the proteins may secrete minimal amounts of proteolytic enzymes.

### Bacterial Consortia

Further provided herein are compositions comprising one or more bacterial species (e.g., a bacterial consortium). A bacterial consortium herein can comprise one or more different bacterial species and/or strains. Such bacterial species and/or strains can belong to one or more different bacterial phyla. Such bacterial phyla can include Verrucomicrobia, Firmicutes, Proteobacteria, Actinobacteria, and/or Bacteroidetes, or a combination thereof. In some instances, a bacterial consortium described herein can comprise one or more Lactobacillus sp. as described herein. The one or more Lactobacillus sp. can include Lactobacillus johnsonii or Lactobacillus crispatus. In such instances, a bacterial consortium herein can comprise one or more Lactobacillus johnsonii or Lactobacillus crispatus strains. Such one or more Lactobacillus crispatus strain(s) can include Lactobacillus crispatus (DSM 33187) (i.e., L. crispatus (DSM 33187)). In various instances, a bacterial consortium herein comprises Lactobacillus crispatus (DSM 33187). In some instances, a bacterial consortium herein can comprise one or more Akkermansia sp. Such one or more Akkermansia sp. can include Akkermansia muciniphila, Akkermansia glycaniphila, or a combination thereof. In some instances, the one or more Akkermansia sp. is Akkermansia muciniphila. In such instances, a bacterial consortium herein can comprise one or more Akkermansia muciniphila strains. Such one or more Akkermansia muciniphila strains can include Akkermansia muciniphila (DSM 33213). In various instances, a bacterial consortium herein comprises Akkermansia muciniphila (DSM 33213). In some instances, a bacterial consortium herein can comprise one or more bacterial species or strains belonging to the phylum Firmicutes. In such instances, the one or more bacterial species or strains can belong to any of the Clostridium clusters, such as Clostridium clusters IV and/or XIVa. Such bacterial consortia can comprise or consist of one or more Faecalibacterium sp. The one or more Faecalibacterium sp. can include Faecalibacterium prausnitzii. In such instances, a bacterial consortium herein can comprise one or more Faecalibacterium prausnitzii strains. Such one or more Faecalibacterium prausnitzii strains can include Faecalibacterium prausnitzii (DSM 33185), Faecalibacterium prausnitzii (DSM 33191), Faecalibacterium prausnitzii (DSM 33186), or Faecalibacterium prausnitzii (DSM 33190), or a combination thereof. In various instances, a bacterial consortium herein comprises Faecalibacterium prausnitzii (DSM 33185).

The bacterial consortia provided herein can comprise, consist essentially of, or consist of 20, 19, 18, 17, 16, 15, 14, 13, 12, 11, 10, 9, 8, 7, 6, 5, 4, 3, 2 or 1 bacterial species and/or strain(s). In some instances, a bacterial consortium comprises or consists of at most 10, 5, 4, or 3 bacterial species. In such instances, a bacterial consortium can comprise or consist of 3 bacterial species. Such consortium can comprise or consist of at least one Lactobacillus sp. The Lactobacillus sp. can be Lactobacillus johnsonii, Lactobacillus rhamnosus, Lactobacillus zeae, Lactobacillus acidipiscis, Lactobacillus acidophilus, Lactobacillus agilis, Lactobacillus aviarius, Lactobacillus brevis, Lactobacillus coleohominis, Lactobacillus crustorum, Lactobacillus crispatus, Lactobacillus curvatus, Lactobacillus diolivorans, Lactobacillus farraginis, Lactobacillus fermentum, Lactobacillus fuchuensis, Lactobacillus harbinensis, Lactobacillus helveticus, Lactobacillus hilgardii, Lactobacillus intestinalis, Lactobacillus jensenii, Lactobacillus kefiranofaciens, Lactobacillus kefiri, Lactobacillus lindneri, Lactobacillus mali, Lactobacillus manihotivorans, Lactobacillus mucosae, Lactobacillus oeni, Lactobacillus oligofermentans, Lactobacillus panis, Lactobacillus pantheris, Lactobacillus parabrevis, Lactobacillus paracollinoides, Lactobacillus parakefiri, Lactobacillus paraplantarum, Lactobacillus pentosus, Lactobacillus pontis, Lactobacillus reuteri, Lactobacillus rossiae, Lactobacillus salivarius, Lactobacillus siliginis, Lactobacillus sucicola, Lactobacillus vaccinostercus, Lactobacillus vaginalis, Lactobacillus vini, Lactococcus garvieae, and/or Lactococcus lactis. A bacterial consortium herein can also comprise or consist of at least one Faecalibacterium sp. In some cases, the Faecalibacterium sp. is Faecalibacterium prausnitzii (or F. prausnitzii). In some instances, a bacterial consortium comprises or consists of Akkermansia sp. Such Akkermansia sp. can be Akkermansia muciniphila.

In some embodiments herein, a bacterial consortium can comprise or consist of Faecalibacterium sp., Lactobacillus sp., and Akkermansia sp. In such instances, a consortium can comprise or consist of the bacterial strains L. crispatus (DSM 33187), A. muciniphila (DSM 33213), and F. prausnitzii (DSM 33185). In some instances, a bacterial consortium can comprise at least about 10⁷ CFU, 5×10⁷ CFU, 10⁸ CFU, 5×10⁸ CFU, 10⁹ CFU, 5×10⁹ CFU, 10⁹ CFU, or 5×10¹⁰ CFU, but no more than about 5×10¹² CFU of a bacterial species or strain. Thus, in some embodiments, provided herein are microbial consortia comprising or consisting of about 5×10⁸ CFU of each of L. crispatus (DSM 33187), A. muciniphila (DSM 33213), and F. prausnitzii (DSM 33185).

### Combination of Binding Agents and Bacterial Consortia

Also provided herein are compositions comprising a binding agent and a microbial consortium. In such cases, the binding agent can be a multivalent Fc-receptor (FcR) binder. Such multivalent FcR binder can be a bivalent Fcγ/Fcε binder as described herein, e.g., an IgG-Fc-IgE-Fc or an IgE-Fc-IgG-Fc construct. In other cases, the multivalent FcR binder can be a trivalent Fcγ/Fcε binder. Such trivalent Fcγ/Fcε binder can comprise two IgG Fc domain, or portions thereof, and one IgE domain, or portion thereof. In some instances, a binding agent can comprise or consist an amino acid sequence encoded by a part of the expression vectors that consist of the nucleotide sequences set forth in SEQ ID NOs: 13-15. Any one or more of such binding agents can be used in combination with a bacterial consortium. Such bacterial consortium can comprise or consist of 1, 2, 3, 4, 5, 6, 7, 8, 9, 10 or more different bacterial strains and/or species as further described herein. Such bacterial species and/or strains can belong to any one or more of the phyla Verrucomicrobia, Firmicutes, Proteobacteria, Actinobacteria, and/or Bacteroidetes. In various instances, a consortium can comprise between 1 and 5 of such bacterial species. In such cases, bacterial species of a consortium can include L. crispatus, A. muciniphila, and/or F. prausnitzii. In such instances, a bacterial consortium can comprise or consist of up to 3 bacterial species. Such consortia can comprise or consist of Faecalibacterium sp., Lactobacillus sp., and Akkermansia sp. In such instances, a consortium can comprise or consist of the bacterial strains L. crispatus (DSM 33187), A. muciniphila (DSM 33213), and F. prausnitzii (DSM 33185). In some instances, a bacterial consortium can comprise at least about 10⁷ CFU, 5×10⁷ CFU, 10⁸ CFU, 5×10⁸ CFU, 10⁹ CFU, 5×10⁹ CFU, 10⁹ CFU, or 5×10¹⁰ CFU, but no more than about 5×10¹² CFU of a bacterial species or strain. Thus, in some embodiments, provided herein are microbial consortia comprising or consisting of about 5×10⁸ CFU of each of L. crispatus (DSM 33187), A. muciniphila (DSM 33213), and F. prausnitzii (DSM 33185).

The compositions provided herein that comprise a chimeric binding agent and one or more bacterial species can be used to treat or reduce the incidence of inflammation in a subject. The inflammation can be allergic inflammation or allergic asthma. The subject can be a human or a rodent.

### Pharmaceutical Compositions

A pharmaceutical composition of the disclosure may be a combination of any protein or chimeric binding agent as described herein with other chemical components, such as carriers, stabilizers, diluents, dispersing agents, suspending agents, thickening agents, antioxidants, solubilizers, buffers, osmolytes, salts, surfactants, amino acids, encapsulating agents, bulking agents, cryoprotectants, and/or excipients. The pharmaceutical composition facilitates administration of a protein described herein to an organism. The pharmaceutical composition may comprise factors that extend the half-life of the protein and/or help the protein to reach their target site(s). A pharmaceutical composition herein can further comprise a microbial consortium comprising one or more bacterial species elsewhere described herein.

Pharmaceutical compositions may be administered in therapeutically-effective amounts as pharmaceutical compositions by various forms and routes including subcutaneous, intramuscular, rectal, aerosol, parenteral, ophthalmic, pulmonary, transdermal, vaginal, optic, nasal, oral, sublingual, inhalation, dermal, intrathecal, intravenous, intratumoral, intranasal, and topical administration. A pharmaceutical composition may be administered in a local or systemic manner, via injection of the protein described herein directly into an organ, optionally in a depot.

Parenteral injections may be formulated for bolus injection or continuous infusion. The pharmaceutical compositions may be in a form suitable for parenteral injection as a sterile suspension, solution or emulsion in oily or aqueous vehicles, and may contain formulatory agents such as suspending, stabilizing and/or dispersing agents. Pharmaceutical formulations for parenteral administration include aqueous solutions of a protein described herein in water-soluble form. Suspensions of protein-antibody complexes described herein may be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. The suspension may also contain suitable stabilizers or agents which increase the solubility and/or reduces the aggregation of such protein-antibody complexes described herein to allow for the preparation of highly concentrated solutions.

Alternatively, the protein described herein may be lyophilized or in powder form for reconstitution with a suitable vehicle, e.g., sterile pyrogen-free water, before use. A purified protein as described herein may be administered intravenously. A protein described herein may be administered to a subject in order to home, target, migrate to, or be directed to a CNS cell, a brain cell, a cancerous cell, or a tumor. A protein as described herein may be conjugated to, linked to, or fused to another protein that provides a targeting function to a specific target cell type in the central nervous system or across the blood brain barrier.

A protein of the disclosure may be applied directly to an organ, or an organ tissue or cells, such as brain or brain tissue or cells, during a surgical procedure. The recombinant protein described herein may be administered topically and may be formulated into a variety of topically administrable compositions, such as solutions, suspensions, lotions, gels, pastes, medicated sticks, balms, creams, and ointments. Such pharmaceutical compositions may contain solubilizers, stabilizers, tonicity enhancing agents, buffers and preservatives.

In practicing the methods of treatment or use provided herein, therapeutically-effective amounts of a protein described herein may be administered in pharmaceutical compositions to a subject suffering from a condition that affects the immune system. The subject may be a mammal such as a human or a primate. A therapeutically-effective amount may vary widely depending on the severity of the disease, the age and relative health of the subject, the potency of the compounds used, and other factors.

A protein as disclosed herein may be cloned into a viral or non-viral expression vector. Such expression vector may be packaged in a viral particle, a virion, or a non-viral carrier or delivery mechanism, which is administered to patients in the form of gene therapy. Patient cells may be extracted and modified to express a protein capable of binding an Fc receptor ex vivo before the modified cells may be returned back to the patient in the form of a cell-based therapy, such that the modified cells will express the protein once transplanted back in the patient.

Pharmaceutical compositions may be formulated using one or more physiologically-acceptable carriers comprising excipients and auxiliaries, which facilitate processing of the active compounds into preparations that may be used pharmaceutically. Formulation may be modified depending upon the route of administration chosen. Pharmaceutical compositions comprising a protein described herein may be manufactured by expressing the protein in a recombinant system, purifying the protein, lyophilizing the protein, mixing, dissolving, granulating, dragee-making, levigating, emulsifying, encapsulating, entrapping, or compression processes. The pharmaceutical compositions may include at least one pharmaceutically acceptable carrier, diluent, or excipient and compounds described herein as free-base or pharmaceutically-acceptable salt form.

Methods for the preparation of protein described herein comprising the compounds described herein include formulating protein described herein with one or more inert, pharmaceutically-acceptable excipients or carriers to form a solid, semi-solid, or liquid composition. Solid compositions may include powders, tablets, dispersible granules, capsules, cachets, and suppositories. These compositions may also contain minor amounts of nontoxic, auxiliary substances, such as wetting or emulsifying agents, pH buffering agents, and other pharmaceutically-acceptable additives.

Non-limiting examples of pharmaceutically-acceptable excipients may be found, for example, in Remington: The Science and Practice of Pharmacy, Nineteenth Ed. (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999), each of which is incorporated by reference in its entirety. Pharmaceutical compositions may also include permeation or absorption enhancers (Aungst et al. AAPS J. 14(1):10-8. (2012) and Moroz et al. Adv Drug Deliv Rev 101:108-21. (2016)). Permeation enhancers may facilitate uptake of molecules from the GI tract into systemic circulation. Permeation enhancers may include salts of medium chain fatty acids, sodium caprate, sodium caprylate, N-(8-[2-hydroxybenzoyl]amino)caprylic acid (SNAC), N-(5-chlorosalicyloyl)-8-aminocaprylic acid (5-CNAC), hydrophilic aromatic alcohols such as phenoxyethanol, benzyl alcohol, and phenyl alcohol, chitosan, alkyl glycosides, dodecyl-2-N,N-dimethylamino propionate (DDAIPP), chelators of divalent cations including EDTA, EGTA, and citric acid, sodium alkyl sulfate, sodium salicylate, lecithin-based, or bile salt-derived agents such as deoxycholates.

The chimeric binding agents (e.g., those comprising an amino acid sequence set forth in any one of SEQ ID NO: 3-SEQ ID NO: 6 or SEQ ID NO: 8-SEQ ID NO: 10 and any combination thereof) may be prepared or formulated for administration (e.g., subcutaneous administration) to a subject by lyophilizing the purified fusion protein (e.g., IgG-Fc-IgE-Fc) followed by reconstitution in a sterile aqueous solution (e.g., sterile water).

The chimeric binding agents of the present disclosure may be formulated as pharmaceutical compositions comprising a certain amount of a chimeric binding agent per volume (e.g., measured in mg/L or mg/mL). A solution comprising a chimeric binding agent may be prepared with a concentration of the chimeric binding agent ranging from about 0.1 mg/mL to about 100 mg/mL. The concentration of a chimeric binding agent in a pharmaceutical composition for administration to a subject in thereof may range from about 1 mg/mL to about 100 mg/mL. The concentration of a chimeric binding agent in a pharmaceutical composition for administration to a subject in thereof may range from about 5 mg/mL to about 75 mg/mL. The concentration of a chimeric binding agent in a pharmaceutical composition for administration to a subject in thereof may range from about 10 mg/mL to about 50 mg/mL. The concentration of a chimeric binding agent in a pharmaceutical composition for administration to a subject in thereof may range from about 15 mg/mL to about 40 mg/mL. The concentration of a chimeric binding agent in a pharmaceutical composition for administration to a subject in thereof may range from about 20 mg/mL to about 30 mg/mL. The concentration of a chimeric binding agent in a pharmaceutical composition for administration to a subject in thereof may be at least about 0.1 mg/mL. The concentration of a chimeric binding agent in a pharmaceutical composition for administration to a subject in thereof may be at least about 1 mg/mL. The concentration of a chimeric binding agent in a pharmaceutical composition for administration to a subject in thereof may be at least about 5 mg/mL. The concentration of a chimeric binding agent in a pharmaceutical composition for administration to a subject in thereof may be at least about 10 mg/mL. The concentration of a chimeric binding agent in a pharmaceutical composition for administration to a subject in thereof may be at least about 20 mg/mL. The concentration of a chimeric binding agent in a pharmaceutical composition for administration to a subject in thereof may be at least about 50 mg/mL. The concentration of a chimeric binding agent in a pharmaceutical composition for administration to a subject in thereof may be at least about 100 mg/mL.

A chimeric binding agent may be administered to a subject in an amount suitable for that particular subject. The suitable amount of chimeric binding agent for administration to a particular subject may be determined using various methodologies including but not limited to blood and immune cell profiling using various cell sorting and single cell analysis methods, e.g., to determine Fc receptor expression and their abundance and distribution on the surfaces of various cell populations in a subject. Amino acid- and/or nucleotide sequencing of certain cells or cell populations may be performed to study the expression of certain markers such as Fc receptors on certain cell populations.

The amount of chimeric binding agent administered to a subject (e.g., a human or a mouse) may range from about 0.01 mg/kg to about 50 mg/kg. The amount of chimeric binding agent administered to a subject may range from about 0.1 mg/kg to about 25 mg/kg. The amount of chimeric binding agent administered to a subject may range from about 1 mg/kg to about 20 mg/kg. The amount of chimeric binding agent administered to a subject may range from about 2.5 mg/kg to about 15 mg/kg. The amount of chimeric binding agent administered to a subject may range from about 2.5 mg/kg to about 10 mg/kg. The amount of chimeric binding agent administered to a subject may range from about 2.5 mg/kg to about 7.5 mg/kg. The amount of chimeric binding agent administered to a subject may range from about 2.5 mg/kg to about 5 mg/kg. The amount of chimeric binding agent administered to a subject may be at least 0.01 mg/kg. The amount of chimeric binding agent administered to a subject may be at least 0.1 mg/kg. The amount of chimeric binding agent administered to a subject may be at least 1 mg/kg. The amount of chimeric binding agent administered to a subject may be at least 2.5 mg/kg. The amount of chimeric binding agent administered to a subject may be at least 5 mg/kg. The amount of chimeric binding agent administered to a subject may be at least 10 mg/kg. The amount of chimeric binding agent administered to a subject may be at least 25 mg/kg.

The therapeutically effective amount of a chimeric binding agent administered to a subject may be from about 0.01 mg/kg to about 25 mg/kg. The therapeutically effective amount of a chimeric binding agent administered to a subject may be from about 0.01 mg/kg to about 10 mg/kg. The therapeutically effective amount of a chimeric binding agent administered to a subject may be from about 0.1 mg/kg to about 15 mg/kg. The therapeutically effective amount of a chimeric binding agent administered to a subject may be from about 1 mg/kg to about 10 mg/kg. The therapeutically effective amount of a chimeric binding agent administered to a subject may be from about 0.1 mg/kg to about 15 mg/kg. The therapeutically effective amount of a chimeric binding agent administered to a subject may be from about 2.5 mg/kg to about 10 mg/kg. The therapeutically effective amount of a chimeric binding agent administered to a subject may be from about 2.5 mg/kg to about 5 mg/kg. The therapeutically effective amount of a chimeric binding agent administered to a subject may be at least about 0.01 mg/kg. The therapeutically effective amount of a chimeric binding agent administered to a subject may be at least about 0.1 mg/kg. The therapeutically effective amount of a chimeric binding agent administered to a subject may be at least about 1 mg/kg. The therapeutically effective amount of a chimeric binding agent administered to a subject may be at least about 2.5 mg/kg. The therapeutically effective amount of a chimeric binding agent administered to a subject may be at least about 5 mg/kg. The therapeutically effective amount of a chimeric binding agent administered to a subject may be at least about 10 mg/kg. The therapeutically effective amount of a chimeric binding agent administered to a subject may be at least about 25 mg/kg.

### Methods of Treatment

The present disclosure provides methods and compositions that may be administered in an effective amount to a subject (e.g., a human or a mouse). The subject may suffer from a disease or conditions, or the subject may be a healthy subject, and administration of one or more chimeric binding agents to the subject treat the disease or conditions and/or prevent a disease or condition such as an allergic disease or condition from occurring or re-occurring in a subject.

The herein described methods and compositions may be used to prevent and/or treat a broad range of diseases and conditions. The herein described methods and compositions may be used to prevent and/or treat an immune disorder in a subject. The immune disorder may cause an inflammatory response in the subject. The herein described methods and compositions may be used to prevent and/or treat such inflammatory response such as an allergic disease, disorder, or condition in a subject. Allergic diseases that may be prevented and/or treated with the presently described methods and compositions may include IgE-mediated hypersensitivity (e.g., Type I), IgG-mediated cytotoxic hypersensitivity (e.g., Type II), immune complex-mediated hypersensitivity (e.g., Type III), and cell-mediated hypersensitivity (e.g., Type IV). The herein described chimeric binding agents may be particularly useful for the prevention and/or treatment of IgE-mediated allergic and/or inflammatory (e.g., allergic inflammation) diseases and conditions. Such conditions may include asthma, allergic asthma, allergic airway inflammation, atopic dermatitis, allergic rhinitis, and several ocular allergic diseases and conditions. The methods and compositions as disclosed herein may be used to prevent and/or treat IgE-mediated (e.g., Type I) allergic diseases and conditions (e.g., atopy). Allergic diseases and conditions that may be treated using the herein described methods and compositions include, but are not limited to, sinusitis, allergic rhinitis, asthma, eczema, hives, food allergies, drug allergies, sting insect allergies, anaphylaxis, urticaria, angioedema (e.g., hereditary angioedema), allergic gastroenteropathy, and seasonal allergies such as hay fever. Seasonal allergies may be prevented and/or treated using a chimeric binding agent as disclosed herein.

The binding of a chimeric binding agent to an FcεR and an FcγR (e.g., the simultaneous binding to an FcεR and an FcγR) may be particularly useful for the prevention and/or treatment of various diseases and conditions such as allergic diseases including IgE-mediated diseases and conditions. Without being bound to any theory, the particular use of the herein disclosed chimeric binding agents may be due to the simultaneous binding to an FcεR in an antagonistic manner (e.g., inhibiting the FcεR) and to an FcγR in an agonistic manner (activating the FcγR), thereby initiating, promoting, or enhancing crosslinking of the FcεR with FcγR and thus ITIM signaling. This mechanism of action may result in an effective suppression of an effector cell such as inhibition of degranulation of mast cells and/or basophils and thus inhibit the excretion of pro-inflammatory molecules (e.g., histamines) from these cells. Furthermore, binding of a chimeric binding agent to an FcR (e.g., an FcεR and/or an FcγR) may prevent an endogenous ligand (such as Ig molecules and Ig-antigen complexes) of such from binding to such receptors. Binding of a chimeric binding agent to an FcεR may prevent an endogenous IgE molecule such as those bound to an antigen from binding to an FcεR.

Generally, allergic diseases that may be treated with chimeric agents as described herein include food allergies, skin allergies, dust allergies, insect sting allergies, pet allergies, eye allergies, drug allergies, allergic rhinitis, latex allergies, mold allergies, sinus infections, and cockroach allergies. In addition, allergic diseases that may be treated with the chimeric agents of the present disclosure include asthma, and chronic urticaria.

Diseases such as urticaria may be prevented and/or treated using a chimeric binding agent as disclosed herein by inhibiting allergen-induced degranulation via antagonistic binding of the FcεR-binding region of the chimeric protein to a FcεR such as FcεRI. Autoimmune diseases that may be mediated by crosslinking of FcεRIs by anti-FCER1A IgG molecules may be prevented and/or treated using the chimeric binding agents of the present disclosure by blocking the binding of auto-reactive antibodies to the FcεRI. Thus, binding of a chimeric binding agent (e.g., IgG-Fc-IgE-Fc protein) to FcεRI and induction of ITIM signaling may be sufficient to effectively suppress autoantibody-induced inflammation.

The chimeric binding agents of the present disclosure may effectively prevent and/or treat Hyper-IgE-Syndrome (i.e., Job's Syndrome) by suppressing chronic mast cell degranulation, a hall mark of this disease which is promoted by extraordinarily high IgE concentrations. Furthermore, administration of a chimeric binding agent as disclosed herein (e.g., an IgG-Fc-IgE-Fc fusion protein) to a subject (e.g., a human or a mouse) may further reduce the risk of viral and bacterial infections and reduced inflammation in lung and skin as confirmed by tissue analysis.

The term “effective amount,” as used herein, generally refers to a sufficient amount of a protein (e.g., a chimeric binding agent or fusion protein) being administered which will relieve to some extent (e.g., completely or partially) one or more of the symptoms of the disease or condition being treated. The result may be reduction and/or alleviation of the signs, symptoms, or causes of a disease, or any other specific alteration of a biological system. Compositions containing proteins and binding agents (e.g., an IgG-Fc-IgE-Fc fusion protein or protein conjugate) may be administered for prophylactic, enhancing, and/or therapeutic treatments. An appropriate “effective” amount in any individual case may be determined using techniques, such as a dose escalation study.

Treatment using the herein described methods and compositions may be provided to the subject before clinical onset of disease. Treatment may be provided to the subject after clinical onset of disease. Treatment may be provided to the subject after 1 day, 1 week, 6 months, 12 months, or 2 years or more after clinical onset of the disease. Treatment may be provided to the subject for more than 1 day, 1 week, 1 month, 6 months, 12 months, 2 years or more after clinical onset of disease. Treatment may be provided to the subject for less than 1 day, 1 week, 1 month, 6 months, 12 months, or 2 years after clinical onset of the disease. Treatment may also include treating a human in a clinical trial. A treatment may comprise administering to a subject a pharmaceutical composition, such as one or more of the pharmaceutical compositions described throughout the disclosure. A treatment may comprise a once daily dosing. A treatment may comprise delivering a chimeric binding agent of the disclosure to a subject subcutaneously, intramuscularly, by inhalation, dermally, topically, by intra-articular injection, orally, sublingually, intrathecally, transdermally, intranasally, via a peritoneal route, directly into a tumor e.g., injection directly into a tumor, directly into the brain, e.g., via and intracerebral ventricle route, or directly onto a joint, e.g., via topical, intra-articular injection route. A treatment may comprise administering a chimeric binding agent-active agent (e.g., drug) complex to a subject, either subcutaneously, intramuscularly, by inhalation, by intra-articular injection, dermally, topically, orally, intrathecally, transdermally, intransally, parenterally, orally, via a peritoneal route, nasally, intravenously, or sublingually.

In some embodiments herein, a chimeric binding agent can be administered in combination with a microbial consortium, wherein the chimeric binding agent and microbial consortium can be administered via the same or different administration routes. In an example, a first pharmaceutical composition comprising a chimeric binding agent can be administered subcutaneously or intravenously, and a second pharmaceutical composition comprising a microbial consortium can be administered orally.

### Experimental Methods

Various techniques and methodologies may be performed to develop chimeric binding agents as described herein. Binding affinities of the chimeric binding agents to one or more receptors may be determined in a concentration-dependent manner and/or in competitive binding experiments.

Expression Vector Construction. The coding regions for murine IgE-Fc proteins and murine chimeric binding agents such as IgG-Fc-IgE-Fc fusion proteins may be cloned into a PiggyBac (PB) expression vector. The PB expression cassette may consist of two promoters, a CMV promoter regulating the expression of the respective Fc protein and an EF1a promoter for constitutive expression of GFP and a puromycin resistance gene (e.g., Puro^{R}). The puromycin resistance may allow positive selection of transfected cells to generate a homogenous cell population for high yield protein production. GFP may be used as an optical readout to analyze the purity of selected cells by flow cytometry. The entire PB expression cassette may be flanked by transposon-specific inverted terminal repeats (ITRs) that may be recognized by a transposase and integrated into the genome of the transfected cells to generate stably expressing cells. The expressed Fc proteins may be secreted into the cell culture medium and purified utilizing chromatography techniques such as size-exclusion chromatography (SEC).

Protein Production and Purification. Amino acid sequences that may be part of a protein library and that may be generated from theoretical and/or in silico design (e.g., computational design), may be synthesized using expression vectors or solid phase or solution phase protein synthesis methods. Proteins such as chimeric binding agents as disclosed herein may be cloned into a secreted, soluble protein production/expression vector and subsequently be purified. Protein purification methods include, but are not limited to, affinity purification columns, ion exchange (cation and/or anion column chromatography), reversed-phase chromatography, hydrophobic interaction chromatography, and size exclusion column chromatography. SDS-PAGE followed by Coomassie staining and reverse phase high-pressure liquid chromatography (HPLC) may be used to analyze a sample of the purified protein. Protein concentrations were determined by UV spectral absorption (e.g., absorbance at 280 nm) and/or amino acid analysis.

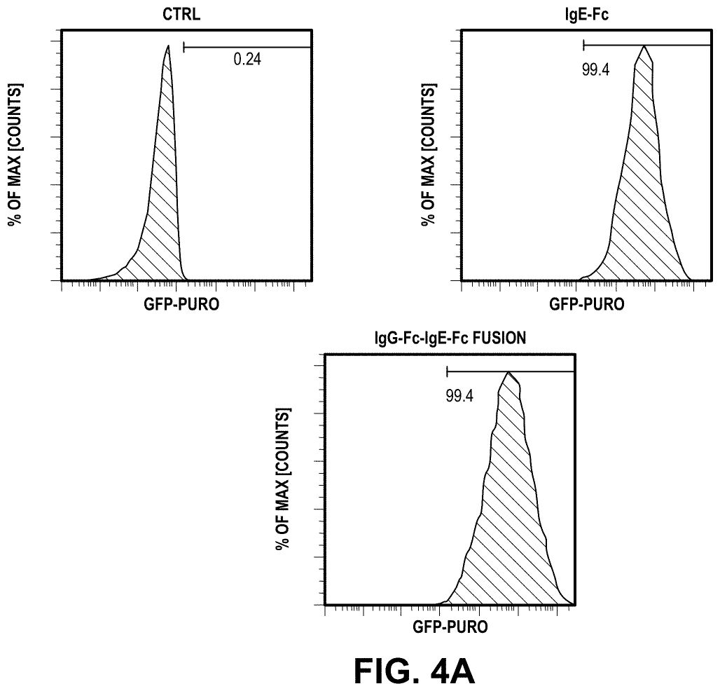

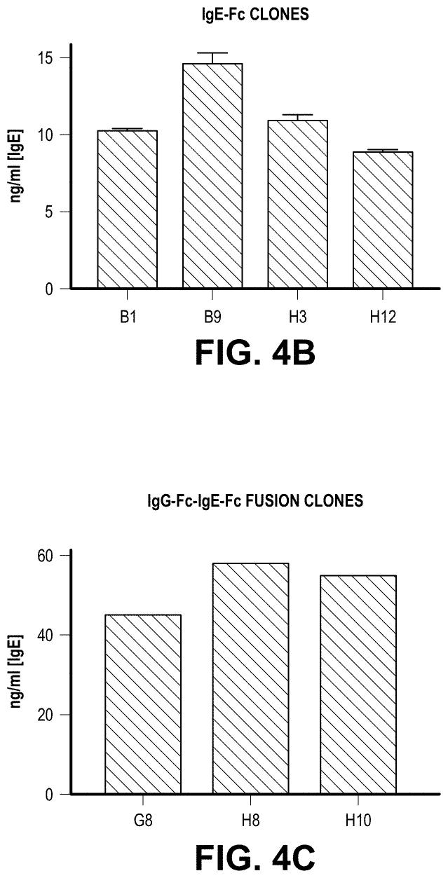

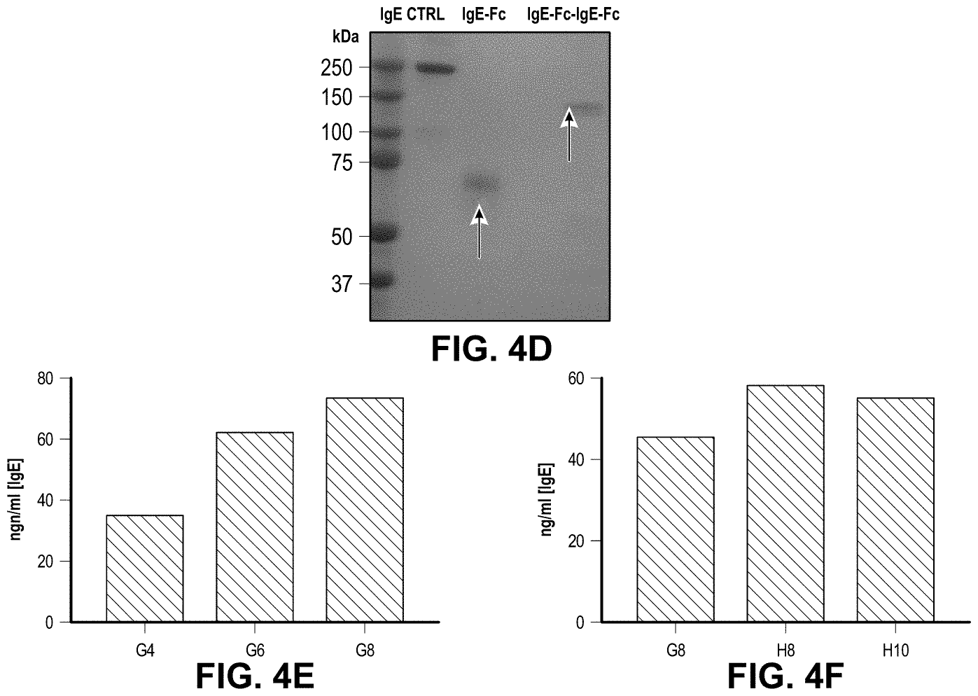

Cell Line Development for High Yield Production of Fc Fusion Proteins. Cell lines for high yield production of the chimeric binding agents as disclosed herein may be developed. To that end, HEK293T cells may be stably transfected with plasmid vectors coding for either the single domain IgE-Fc or chimeric fusion protein such as a IgG-Fc-IgE-Fc protein. Transfected cells may be positively selected with puromycin to generate a stable expression cell line. Plasmid vectors may also carry GFP which may be used to analyze the purity of the puromycin-selected cell lines by flow cytometry. After several (e.g., 12) days of selection, in both cell lines fusion proteins such as IgG-Fc-IgE-Fc may be produced in over 99% (e.g., 99.4%) of cells, as may be shown in the respective culture by cells characterized as positive for the expression cassette (FIG. 4A). The selected cells may be sub-cloned and the clones with the highest mean fluorescence intensity of GFP may be selected and further expanded. These candidates may be then screened for protein production levels in the cell culture supernatants by ELISA. The clones producing the highest yield of IgE-Fc and IgG-Fc-IgE-Fc, respectively, may be again selected, expanded and used for drug production. The fusion proteins or chimeric binding agents produced in high yield by the selected sub-clones may be confirmed by Coomassie blue staining.

Fluorophore Conjugation. Screening proteins or a library of proteins may involve labeling of a protein of interest or a protein partner with a fluorophore or any other detectable moiety, such that detection of a signal from the fluorophore or detectable moiety may be indicative of binding to the protein. An example of a fluorophore that may be used is Alexa Fluor 647 NHS Ester (Life Technologies), which may be used to label a protein of interest as per manufacturer's protocol. Saturation may be approximately 1-5 fluorophore per molecule, as determined by mass spectrometry.

General Determination of Protein Uptake in Cells or Tissues. The uptake of a protein or protein construct as disclosed herein may be determined in a specific cell, cell population, tissue, or organ either ex vivo or in vivo. In the same way, the efficiency of cargo or payload delivery may be determined ex vivo or in vivo in cases where the cargo molecule comprises a detectable agent. Ex vivo analyses include organ harvest and fixation (e.g., using 4% formaldehyde) of harvested tissue before analyses. Tissue samples may be analyzed using a variety of analytical methods including microscopy, spectroscopy, flow cytometry, polymerase chain reaction (PCR), and via measurements of ultrasound, electromagnetic radiation (e.g., UV/VIS, X-ray) or radioactivity. Tissue and/or organ uptake may be determined by measuring luminescence or bioluminescence of a cell, cell population, tissue, or organ sample, or by measuring radioactivity of a cell, cell population, tissue, or organ sample and by calculating uptake values such as percent injected dose per gram (or per mole or per volume). In addition, the acquisition of nuclear images visualizing the biodistribution at a specific point in time (e.g., static imaging) or over a time course (e.g. dynamic imaging) may be performed using various techniques (e.g., PET or SPECT) and appropriately radiolabeled proteins.

Binding Affinity of Expressed Fusion Proteins to Fc Receptors. The histamine-releasing rat cell line RBL-2H3 and human peripheral blood mononuclear cells (PBMCs) may be used and treated with IgE-Fc (purified) and IgG-Fc-IgE-Fc (unpurified, obtained from cell culture supernatant) for 30 min. Cells may then be thoroughly washed and stained with APC conjugated anti-His antibodies and subsequently analyzed by flow cytometry. Anti-His antibodies may be used to detect Fc proteins (e.g., chimeric binding agents) that may be bound to the cell surface due to their specific interaction with Fc receptors such as Fc epsilon Receptor I and Fc gamma Receptor IIB, respectively. IgG-Fc-IgE-Fc proteins may be able to bind to both the rat basophil cell line RBL-2H3 as well as to human PBMC, even in very low concentrations in which the IgG-Fc-IgE-Fc fusion protein may be present in cell culture supernatants compared to purified and highly concentrated single region IgE-Fc proteins.

Protein Resistance to Proteases. The stability of proteins of this disclosure may be determined by resistance to degradation by proteases. Proteases, also referred to as peptidases or proteinases, are enzymes that may degrade proteins and proteins by breaking bonds between adjacent amino acids. Families of proteases with specificity for targeting specific amino acids may include serine proteases, cysteine proteases, threonine proteases, aspartic proteases, glutamic proteases, and asparagine proteases. Additionally, metalloproteases, matrix metalloproteases, elastase, carboxypeptidases, Cytochrome P450 enzymes, and cathepsins may also digest proteins and proteins. Proteases may be present at high concentration in blood, in mucous membranes, lungs, skin, the GI tract, the mouth, nose, eye, and in compartments of the cell. Misregulation of proteases may also be present in various diseases such as rheumatoid arthritis and other immune disorders. Degradation by proteases may reduce bioavailability, biodistribution, half-life, and bioactivity of therapeutic molecules such that they may be unable to perform their therapeutic function. Proteins as described herein that may be resistant to proteases may better provide therapeutic activity at reasonably tolerated concentrations in vivo.

### EXAMPLES

The following examples are included to further describe certain aspects of the present disclosure, and should not be used to limit the scope of the disclosure.

### Example 1

### Construction of Expression Vectors for Murine IgE-Fc and Murine IgG-Fc-IgE-Fc Binding Agents

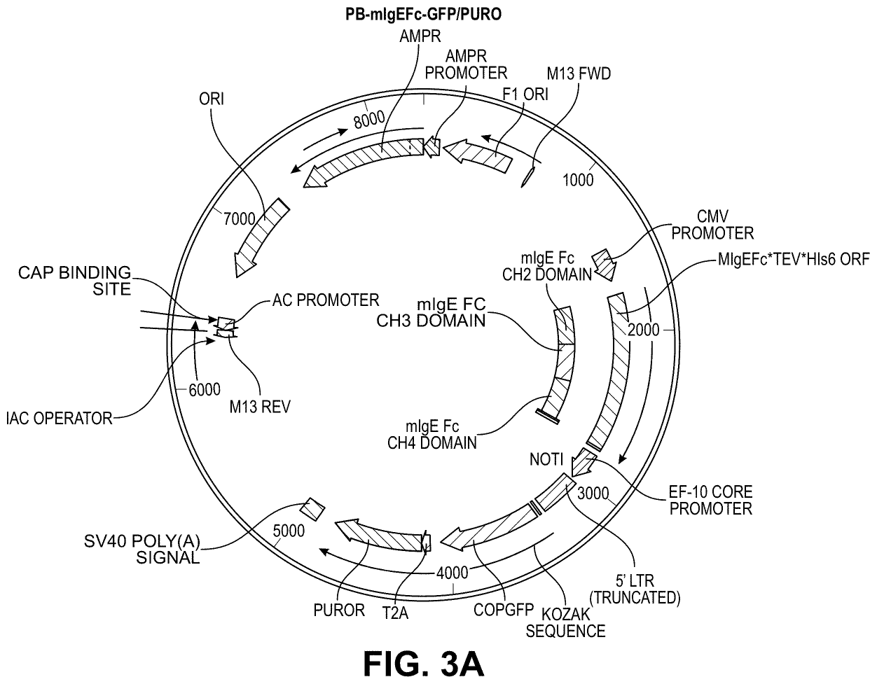

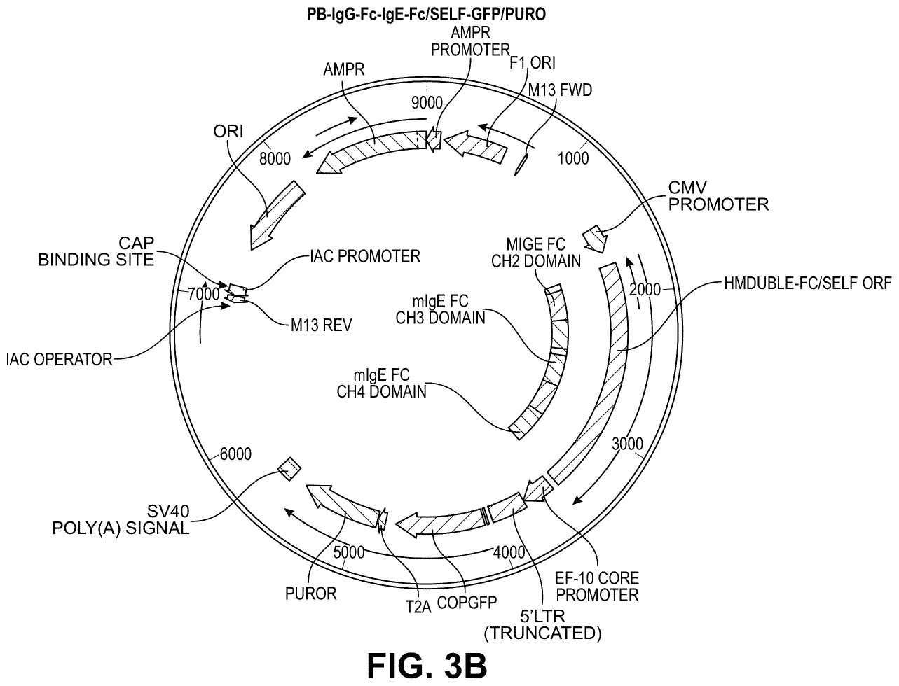

This example demonstrates the design and construction of expression vectors for murine IgE-Fc and murine IgG-Fc-IgE-Fc fusion proteins (FIG. 3).

The coding regions for murine IgE-Fc proteins (FIG. 3A) and murine IgG-Fc-IgE-Fc fusion proteins (FIG. 3B) were cloned into a PiggyBac (PB) expression vector. The PB expression cassette contained two promoters, a CMV promoter regulating the expression of the respective Fc protein and an EF1a promoter for constitutive expression of GFP and a puromycin resistance gene (Puro^{R}). The puromycin resistance allows positive selection of transfected cells to generate a homogenous cell population for high yield protein production. GFP was used as an optical readout to analyze the purity of the selected cells by flow cytometry. The entire PB expression cassette was flanked by transposon-specific inverted terminal repeats (ITRs) that are recognized by a transposase and integrated into the genome of the transfected cells to generate stably expressing cells.

The expressed Fc proteins were secreted into the cell culture medium and purified utilizing chromatography techniques such as SEC.

### Example 2

### Cell Line Development for High Yield Production of Chimeric Binding Agents

This example demonstrates the development of cell lines for high yield production of murine and human chimeric binding agents (e.g., those multivalent binding agents depicted in FIGS. 1A-1G), as well as their purification and analysis (FIG. 4).

For the production of murine Ig-Fc and Ig-Fc fusion proteins, HEK293T cells were stably transfected with plasmid vectors coding for either the single domain IgE-Fc (e.g., SEQ ID NO: 5) or IgG-Fc-IgE-Fc (e.g., SEQ ID NO: 12). Transfected cells were positively selected with puromycin to generate a stable expression cell line. Plasmid vectors also carried GFP, which was used to analyze the purity of the puromycin-selected cell lines by flow cytometry. After 12 days of selection, in both cell lines (IgE-Fc and IgG-Fc-IgE-Fc), 99.4% of cells in the respective culture were positive for the expression cassette (FIG. 4A).

The selected cells were then sub-cloned and the clones with the highest mean fluorescence intensity of GFP were selected and further expanded (FIGS. 4B-4C). These candidates were then screened for protein production levels in the cell culture supernatants by ELISA. The clones producing the highest yield of IgE-Fc and IgG-Fc-IgE-Fc, respectively, were again selected, expanded, and used for drug production.

The fusion proteins produced by the selected sub-clones were confirmed by Coomassie blue staining. FIG. 4D shows that the size and integrity of the purified proteins under non-reducing conditions for IgE-Fc (˜70 kDa) and the IgG-Fc-IgE-Fc fusion (˜135 kDa) matched the predicted molecular size of the produced fusion proteins confirming successful selection of cell lines that stably produced the fusion proteins in high yields. The chimeric binding agents (comprising a TEV-His tag) were purified using Ni-NTA beads followed by the elution of the chimeric binding agents from beads using chromatography with imidazole.

For the production of fully human Ig-Fc and Ig-Fc fusion proteins, HEK293T cells were stably transfected with plasmid vectors comprising nucleotide sequences coding for IgG-IgE-Fc or IgE-IgG-Fc chimeric binding agents, e.g., those nucleotide sequences set forth in SEQ ID NOs: 13-15, or any other nucleotide sequence described herein. Transfected cells were positively selected with puromycin to generate a stable expression cell line. Plasmid vectors also carried GFP, which was used to analyze the purity of the puromycin-selected cell lines by flow cytometry. After 12 days of selection, in both cell line cultures (IgE-Fc- and IgG-Fc-IgE-Fc-producing cultures) nearly 100% of cells in the respective culture were positive for the expression cassette. Subsequently, the selected cells were sub-cloned and the clones with the highest mean fluorescence intensity of GFP were selected and further expanded (FIG. 4E). These candidates were then screened for protein production levels in the cell culture supernatants by ELISA. The clones producing the highest yield of binding agent were again selected, expanded, and used for drug production.

Cell line development for the production of chimeric binding agents comprising an IgG-Fc portion comprising a S267E and a L328F (i.e., “SELF”) or an E333A substitution was conducted using similar methods as described above in this example for chimeric binding agents comprising IgG-Fc murine or human wild-type regions. FIG. 4E shows the production yield (in ng/mL) after 4 days in the selected G8, H8, and H10 cell lines for the binding agent containing the amino acid sequence set forth in SEQ ID NO: 12.

These data demonstrates that the chimeric binding agents described herein can be produced in high yields (e.g., at least about 10, 20, or 40 ng/mL cell supernatant) and purified to high chemical purities (e.g., >90%).

### Example 3

### Evaluation of Binding Affinity of Chimeric Binding Agents to Fc Receptors

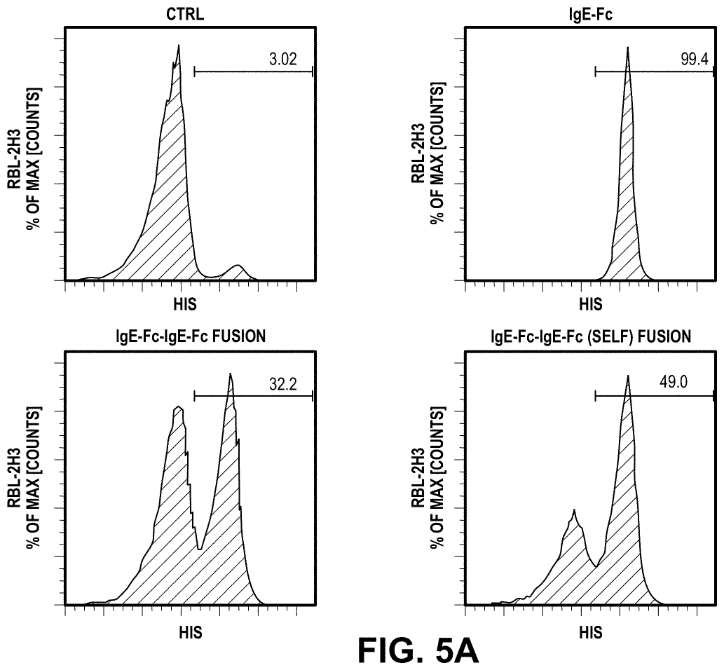

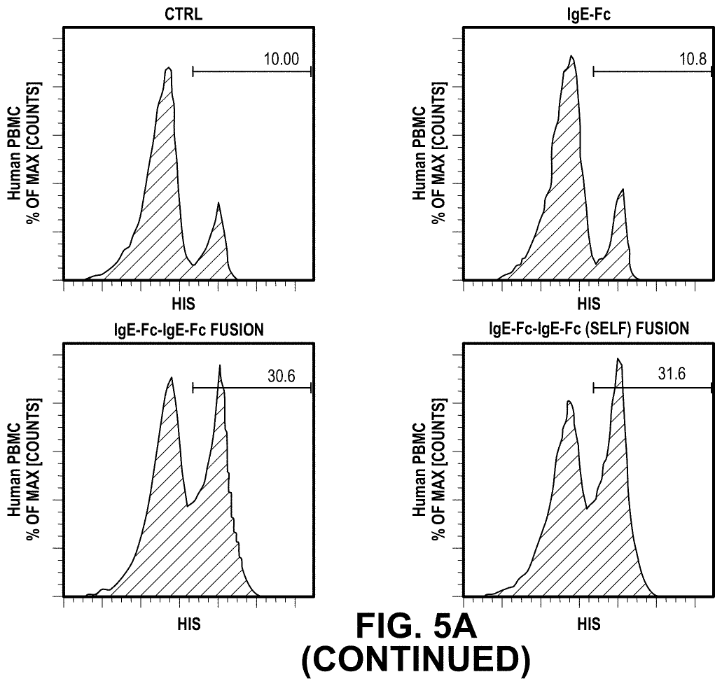

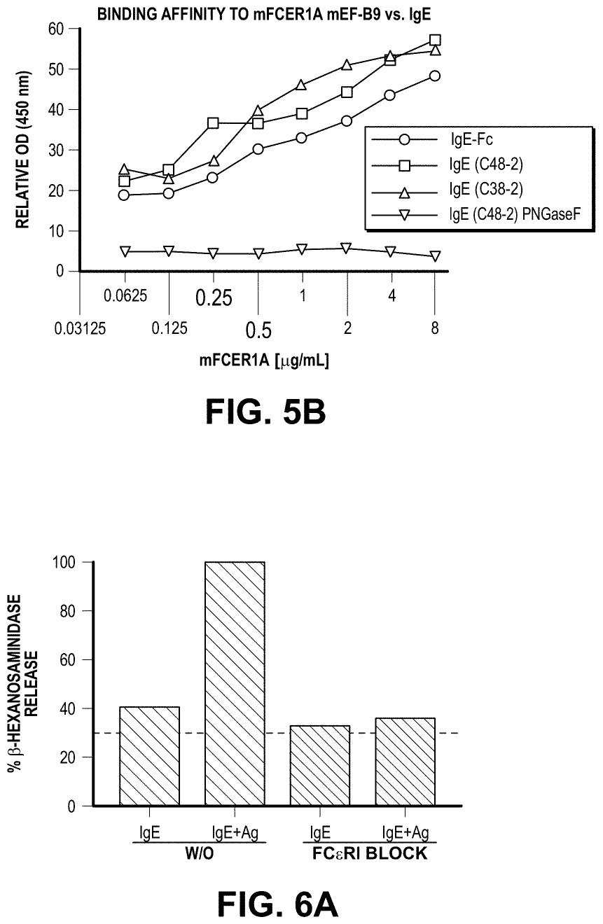

This example demonstrates the determination of binding affinity of the expressed binding agents IgE-Fc (e.g., SEQ ID NO: 5) and IgG-Fc-IgE-Fc (e.g., SEQ ID NO: 12, comprising the SELF substitution, and those containing wild-type IgG portions) to Fc Receptors (FIGS. 5A-5B).

Cells of the histamine-releasing rat cell line RBL-2H3 and human peripheral blood mononuclear cells (PBMCs) were treated with the IgE-Fc protein (purified) having the amino acid sequence set forth in SEQ ID NO: 5 and the IgG-Fc-IgE-Fc fusion protein (unpurified, obtained from cell culture supernatant) having the amino acid sequence set forth in SEQ ID NO: 12 for 30 min. Cells were then thoroughly washed and stained with allophycocyanin (APC) conjugated anti-His antibodies and subsequently analyzed by flow cytometry. Anti-His antibodies detected Fc proteins that were bound to the cell surface due to their specific interaction with Fc epsilon Receptor I (FcεRI) and Fc gamma Receptor IIB (FcγRIIB), respectively. IgG-Fc-IgE-Fc proteins were able to bind to both the rat basophil cell line RBL-2H3 as well as to human PBMCs (comprised about 50-70% neutrophils, 10-25% T cells, and 20-30% of cells expressing FcgRIIB), even in very low concentrations in which the IgG-Fc-IgE-Fc fusion protein was present (approximately 0.05 mg/mL) in cell culture supernatants compared to purified and highly concentrated IgE-Fc proteins (approximately 1 mg/mL). This demonstrated the high binding affinity of the IgG-Fc-IgE-Fc fusion protein to both human and murine Fc epsilon Receptor I and Fc gamma Receptor IIB (FIG. 5A).

FIG. 5B shows the binding affinity of purified IgE-Fc protein to mouse Fc epsilon receptor 1 a-chain (mFCER1A) as analyzed by ELISA. Plates were coated with increasing concentrations of mFCER1A protein and probed with IgE-Fc protein. Binding affinity of the IgE-Fc protein to the Fc epsilon receptor was compared to commercially available IgE antibodies (clone C48-2 and clone C38-2) which served as positive controls. PNGaseF-treated deglycosylated IgE clone C48-2 served as negative control.

These data demonstrate high affinity interactions of the chimeric binding agent consisting of the amino acid sequence set forth in SEQ ID NO: 12 with the Fc receptors FcεRI and FcγRIIB, and further show that the IgE-Fc regions of chimeric binding agents exhibit a binding affinity to FcεRs that is at least as high, if not higher, than that of full-length IgE antibodies.

### Example 4

### Suppression of IgE-Promoted Allergic Inflammation Using a Chimeric Binding Agent

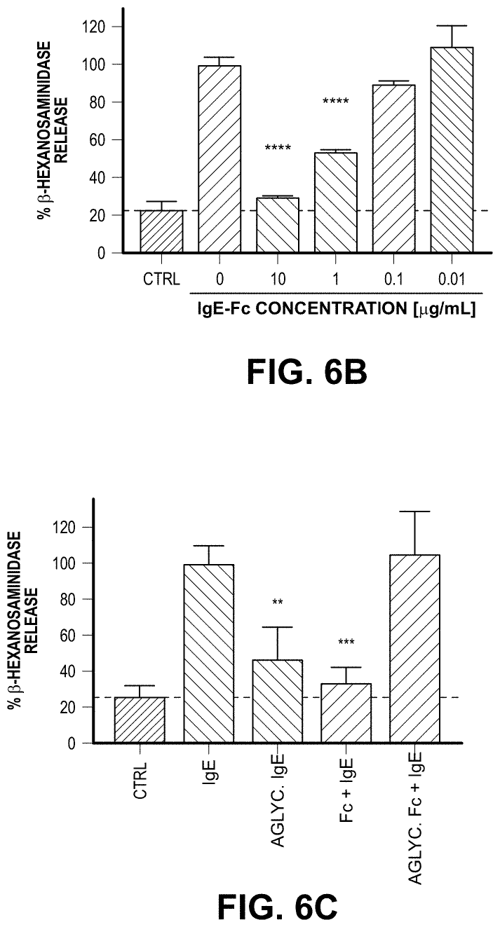

This example demonstrates that blocking of Fc receptors using the chimeric binding agents described herein that contain one or more IgE-Fc regions can suppress IgE-mediated allergic inflammation (FIG. 6).

For these studies, rat RBL-2H3 cells were sensitized with 1 μg/ml TNP-specific IgE and challenged with 2 μg/ml antigen (TNP-KLH), comprising a 2,4,6-trinitrophenyl hapten conjugated to KHL protein via lysine conjugation.

First, and before sensitization with IgE, cells were either treated with PBS (black bars) or with 1 μg/ml non-specific IgE to saturate and block Fc epsilon receptors (grey bars). Pre-treatment efficiently inhibited IgE-mediated degranulation by measuring beta-hexosaminidase release into the extracellular medium after challenge with the TNP-KLH antigen (abbreviated here as “Ag”) (FIG. 6A).

Next, rat RBL-2H3 cells received a pre-treatment of 16 hours with increasing concentrations of purified IgE-Fc (SEQ ID NO: 5) before sensitization and antigen challenge. Complete inhibition of degranulation was observed with equimolar concentrations of IgE-Fc and antigen. This inhibitory effect occurred in a dose-dependent manner, indicating that full saturation of Fc receptors may block antigen-specific IgE antibodies from binding and activating the cells (FIG. 6B).

In a follow-up experiment, cells were either pre-treated with PBS, IgE-Fc or aglycosylated IgE-Fc. As shown in FIG. 6C, sensitization was achieved by incubating the cells with antigen-specific IgE or aglycosylated antigen-specific IgE and subsequently challenged with antigen. Induction of degranulation occurred when antibodies were fully glycosylated but showed significantly decreased potential when aglycosylated. The same effect was observed for pre-treated cells, which were protected from IgE-mediated degranulation when treated with fully glycosylated IgE-Fc but showed significantly decreased potential when aglycosylated IgE-Fc was used.

FIG. 6C demonstrates that induction of degranulation was measured efficiently when antibodies were fully glycosylated but was significantly reduced when the antibodies were aglycosylated. FIG. 6C further illustrates the same effect for pre-treated cells, which were protected from IgE-mediated degranulation when treated with fully glycosylated IgE-Fc but lost protection when aglycosylated IgE-Fc was used. This together indicates that both antigen-specific antibodies as well as potential antibody-derived FcR antagonists may have to be properly glycosylated in order to be able to interact with Fc receptors.

These data indicate that both antigen-specific antibodies as well as potential antibody-derived FcR antagonists, when glycosylated strongly interact with Fc receptors. These results may have further implications for protein production as correct glycosylation states may have to be monitored to ensure adequate protein function.

### Example 5

### In Vitro Degranulation Assays of Mast Cells and Basophils Using Chimeric Binding Agents

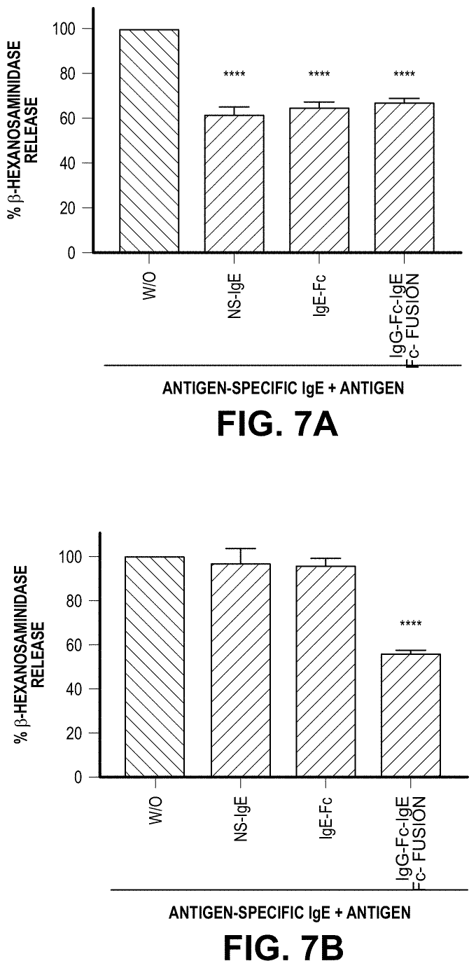

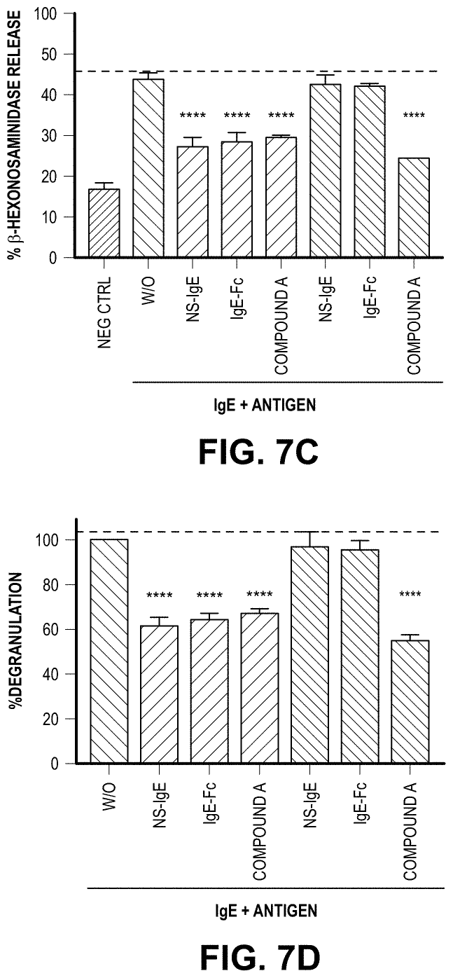

This example demonstrates that blocking of Fc receptors using the chimeric binding agents described herein that contain one or more IgE Fc domains can suppress IgE-mediated allergic inflammation by reducing mast cell and basophil degranulation (FIGS. 7A-7B).

RBL-2H3 cells were sensitized with 1 μg/ml TNP-specific IgE and challenged with 2 μg/ml antigen (TNP-KLH). The chimeric binding agents IgE-Fc and IgG-Fc-IgE-Fc were used in equimolar concentrations.

For the first experiment (FIG. 7A), RBL-2H3 cells were pre-treated with PBS (w/o), non-specific IgE (NS-IgE) or the fusion proteins IgE-Fc (consisting of SEQ ID NO: 5) and IgG-Fc-IgE-Fc (consisting of SEQ ID NO: 12). Cells were incubated overnight and then challenged with antigen-specific IgE. Following antigen challenge, β-hexosaminidase release in the supernatant was quantified and used as a measure of degranulation. All proteins used for pre-treatment that were capable of binding to FcεRs prevented cell activation and degranulation in the presence of antigens effectively, as shown by the grey graphs in FIG. 7A. These data demonstrate that both chimeric binding agents IgE-Fc and IgG-Fc-IgE-Fc were effective in preventing target cell degranulation in a preventative treatment model.

In a follow-up experiment (FIG. 7B), cells received either the Ns-IgE or the chimeric binding agents IgE-Fc (SEQ ID NO: 5) and IgG-Fc-IgE-Fc (SEQ ID NO: 12, compound A) simultaneously with antigen-specific antibodies; thus, Ns-IgE and the fusion proteins IgE-Fc and IgG-Fc-IgE-Fc were competing with antigen-specific antibodies for the FcR binding sites. Following two hours of incubation, cells were challenged with antigen. Degranulation occurred effectively in the positive control (w/o) that did not receive any FcR antagonists or chimeric binding agents. Cells simultaneously treated with antigen-specific IgE and NS-IgE or IgE-Fc, respectively, were not protected from degranulation, because the FcεRs were not fully occupied and thus accessible for antigen-specific IgE. However, the chimeric binding agent consisting of the amino acid sequence set forth in SEQ ID NO: 12 effectively prevented degranulation, presumably due to its ability to bind to both Fc receptors FcεRI as well as FcγRIIB

Subsequently, the ability of the chimeric binding agents IgE-Fc (SEQ ID NO: 5) and IgG-Fc-IgE-Fc (SEQ ID NO: 12, compound A) to protect RBL-2H3 cells against degranulation in a preventative and a therapeutic setting was evaluated. FIG. 7C shows the percent beta-hexanosaminidase release in a preventative (bar nr. 3-5) and a therapeutic (bar nr. 6-8) setting in which the fusion proteins IgE-Fc (SEQ ID NO: 5) and IgG-Fc-IgE-Fc (SEQ ID NO: 12, compound A) are administered after antigen challenge. FIG. 7D shows the percent degranulation of RBL-2H3 cells in a preventative (bar nr. 3-5) and a therapeutic (bar nr. 6-8) setting in which the fusion proteins IgE-Fc (SEQ ID NO: 5) and IgG-Fc-IgE-Fc (SEQ ID NO: 12, compound A) are administered after antigen challenge. These data demonstrate the ability of the bivalent IgG-Fc-IgE-Fc binding agent to protect against effector cell degranulation in both preventative as well as therapeutic settings, likely due to the constructs ability to engage FcγRIIB and its co-ligation with FcεRI.

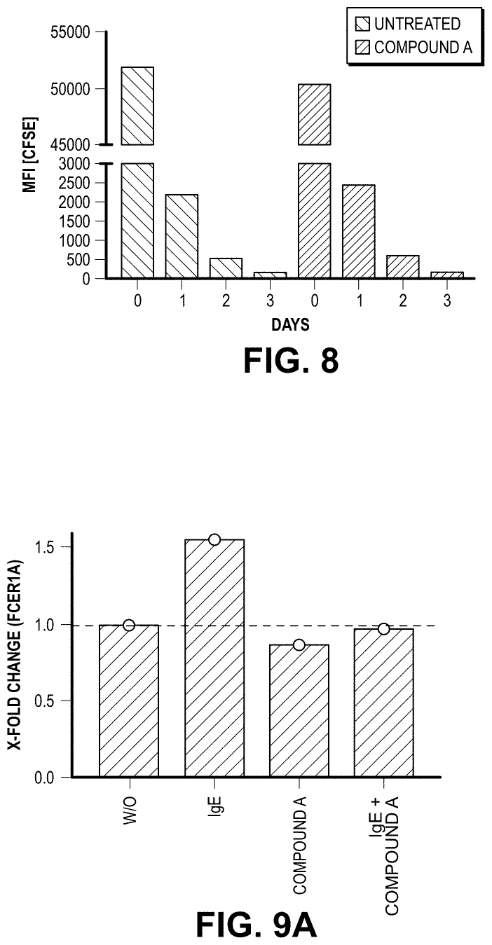

Moreover, FIG. 8 shows that the bivalent binding agent with the amino acid sequence set forth in SEQ ID NO: 12 does not affect the viability and proliferation of Rat Basophilic Leukemia (RBL) cells when used in concentrations of 10 ng/mL protein. Cell viability and proliferation was analyzed for 3 days post treatment. Data are shown as mean fluorescence intensity (MFI) using the cytoplasmic dye carboxyfluorescein diacetate succinimidyl ester (CFSE).

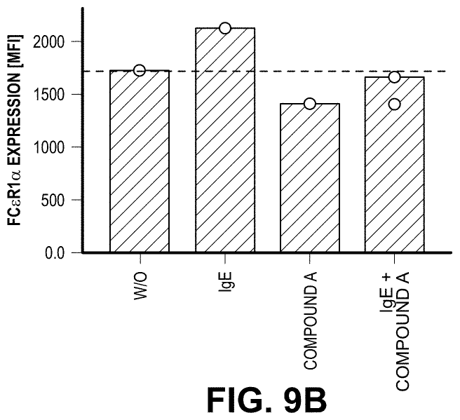

Subsequently, it was demonstrated that the bivalent IgG-Fc-IgE-Fc binding agent consisting of SEQ ID NO: 12 prevented IgE-mediated upregulation of FcεRIa expression in RBL-2H3 cells. FIGS. 9A-9B show that the chimeric binding agent blocked IgE-mediated upregulation of FcεRIa expression, demonstrating the ability of the chimeric binding agents provided herein to prevent and/or treat inflammation, particularly allergic inflammation.

Together, these data demonstrate that degranulation and Fc receptor expression in cells treated with the IgG-Fc-IgE-Fc chimeric binding agent (SEQ ID NO: 12, compound A) were significantly suppressed, which was likely due to the binding agent's ability to selectively co-engage the inhibitory receptors FcγRIIB and FCεRI. This co-ligation of FcγRIIB (activation of inhibitory functions) and FcεRI (blocking of activating functions) can trigger a negative signal cascade through ITIM signaling that can result in inactivation and hence suppression of degranulation of target cells such as mast cells and basophils.

### Example 6

### ADCC Assays Using Chimeric Binding Agents

This example demonstrates that bivalent chimeric binding agents disclosed herein can deplete FceRI+ cells via antibody-dependent cell-mediated cytotoxicity (ADCC) and/or complement dependent cytotoxicity (CDC).

For this study, 5×10⁴ RBL-2H3 cells per well in a 48-well plate were incubated at room temperature until cell count doubled to 10⁵ cells per well. The ADCC-enhanced binding agent consisting of the amino acid sequence set forth in SEQ ID NO: 28 (compound B) was incubated with either (i) RBL-2H3 cells alone or (ii) RBL-2H3 cells and 5×10⁴ PBMC cells. The binding agent consisting of the amino acid sequence set forth in SEQ ID NO: 28 (compound B) comprises a human IgE-Fc region that is coupled via its C-terminus to a human IgG-Fc region, and wherein the IgE region is coupled to the IgG-Fc region via (G₄S)₃ linker. The IgG-Fc region is a wild-type human IgE-Fc portion that contains a single E333A substitution. Such substitution can enhance ADCC and CDC activity of the binding agent compared to binding agent not comprising such substitution.

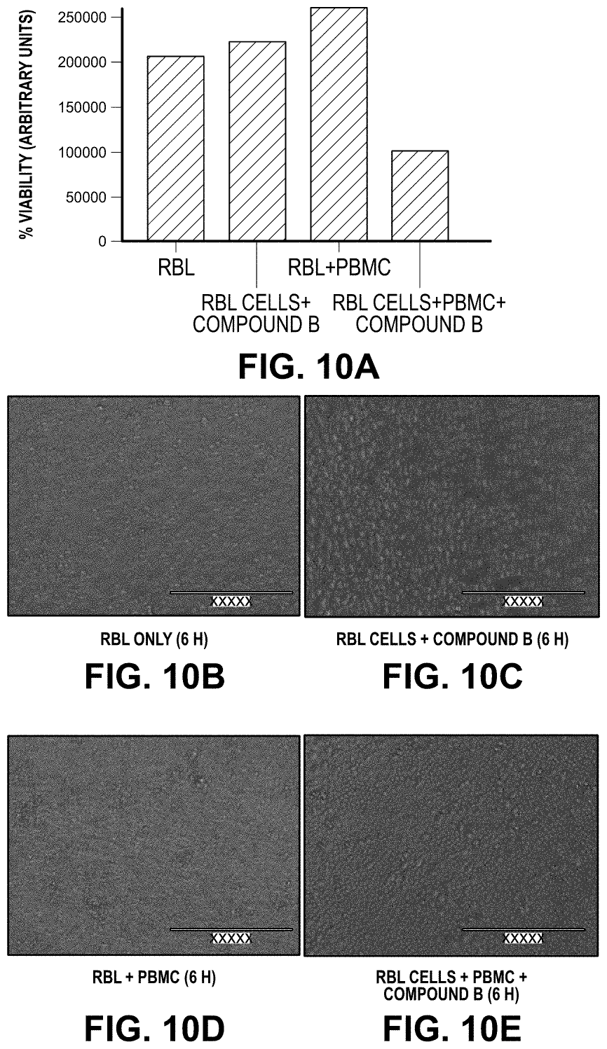

The data demonstrate that the binding agent in the presence of PBMC effector cells facilitated depletion of FceRI+ cells via ADCC by about 50% within 6 hours, as shown in FIGS. 10A-10E. This suggests that the chimeric binding agent with SEQ ID NO: 28 (compound B) induced cell death of FceRI+ cells mediated by PBMC effector cells induced by ADCC and CDC.

Taken together, these results demonstrate that the chimeric binding agents disclosed herein can be used for depletion of FceRI+ cells via ADCC in the presence of effector cells (e.g., PBMCs). This can be an additional mechanism by which a chimeric binding agent of this disclosure herein can reduce inflammation and the potential for allergic reactions in a subject. The other at least 3 mechanisms of action by which a binding agent herein can act on FceRI+ cells are (i) inhibition of upregualtion of FceRI expression on effector cells, (ii) prevention of IgE binding to FceRI by occupying the receptors; and (iii) inactivating target cells (e.g., mast cell and basophils) through co-ligation of FceRI and FcgRIIB

### Example 7

### In Vivo Model to Investigate Anti-Inflammatory Properties of Chimeric Binding Agents

This example demonstrates the effectiveness of pre-treating mice with IgG-Fc-IgE-Fc fusion proteins before antigen challenge to suppress IgE-mediated allergic inflammation in vivo.

Immune competent mice are treated with different doses of the chimeric binding agent IgG-Fc-IgE-Fc ranging from 2.5 mg/kg to 10 mg/kg at various time points before antigen challenge in order to investigate the pharmacokinetic of the IgG-Fc-IgE-Fc fusion proteins and the pharmacodynamic of FcR interaction and potential effective receptor blockage.

### Example 8

### In Vivo Model to Investigate Anti-Inflammatory Properties of Chimeric Binding Agents

This example demonstrates treatment of mice with IgG-Fc-IgE-Fc fusion proteins simultaneously with antigen-specific IgE to compete for binding to FcεRs in order to treat and/or suppress IgE-mediated diseases.

Mice are administered an antigen-specific IgE and a chimeric binding agent (e.g., IgG-Fc-IgE-Fc fusion protein) simultaneously, which then compete for binding to FcεRs in vivo. Target cells in blood are analyzed at specific time points following administration to evaluate binding of antigen-specific IgE and chimeric binding agent to FcεRs on the surface of target cells (e.g., mast cells or basophils).

### Example 9

### In Vivo Model to Investigate Pharmacokinetic Properties of IgG-Fc-IgE-Fc Fusion Proteins

This example demonstrates an investigation of the pharmacokinetic properties of chimeric binding agents as described in the present disclosure.

Mice are administered with chimeric binding agents via the lateral tail vein. Serum-half-life of the constructs is determined using blood draw at various time points post administration (e.g., 5, 10, 15, 30, 45, 60, 120, 240 min and up to several days after administration). In addition, various tissues are analyzed at multiple time points to determine durability of Fc receptor interaction and target engagement.

### Example 10

### In Vivo Model to Investigate Combination Therapies Comprising Chimeric Binding Agents

This example demonstrates the investigation of combination therapies comprising administering to a subject (e.g., a human or a rodent) one or more chimeric binding agents and one or more other drugs of other drug classes for the prevention and treatment of IgE-mediated diseases. The chimeric binding agents are tested in combination with different treatment options in order to maximize effectiveness in vivo and breadth of potential indications. In this case, the additional drug for use in a combination therapy is a bacterial consortium. Such consortium can contain the bacterial species Lactobacillus crispatus, Faecalibacterium prausnitzii, and Akkermansia muciniphila, amongst others.

For an in vivo study, mice are administered with chimeric binding agents via the lateral tail vein either alone, or in combination with the bacterial consortium. The experimental cohorts receiving the drug combination show signs of faster and more sustained anti-inflammatory effects and fewer allergic reactions after sensitization and antigen challenge.

### Example 11

### Treatment of Urticaria Using Chimeric Binding Agents

This example demonstrates the treatment of urticaria in a subject (e.g., a mouse or a human) using the herein disclosed chimeric binding agents (e.g., an IgG-Fc-IgE-Fc fusion protein).

Mice are administered with an IgG-Fc-IgE-Fc fusion protein via the lateral tail vein. Mice from the experimental and control cohorts are monitored over several weeks and their disease progression is assessed. Approximately 10 weeks after treatment, the experimental cohorts that received the chimeric binding agent show significantly improved health and vital signs compared to the control mice that received a saline solution or antihistamines but no chimeric binding agent.

### Example 12

### Treatment of Seasonal Allergies Using Chimeric Binding Agents

This example demonstrates the treatment of seasonal allergies in a subject (e.g., a mouse or a human) using the herein disclosed chimeric binding agents (e.g., an IgG-Fc-IgE-Fc fusion protein).

Mice are administered with an IgG-Fc-IgE-Fc fusion protein via the lateral tail vein. Mice from the experimental and control cohorts are monitored over several weeks and their disease progression is assessed. Approximately 10 weeks after treatment, the experimental cohorts that received the chimeric binding agent are analyzed for improved health and vital signs compared to the control mice that received a saline solution or antihistamines but no chimeric binding agent.

### Example 13

### Treatment of Hyper-IgE-Syndrome (Job's Syndrome) Using Chimeric Binding Agents

This example demonstrates the treatment of seasonal allergies in a subject (e.g., a mouse or a human) using the herein disclosed chimeric binding agents (e.g., an IgG-Fc-IgE-Fc fusion protein).

Mice are administered with an IgG-Fc-IgE-Fc fusion protein via the lateral tail vein. Mice from the experimental and control cohorts are monitored over several weeks and their disease progression is assessed. Approximately 10 weeks after treatment, the experimental cohorts that received the chimeric binding agent show significantly improved health and vital signs compared to the control mice that received a saline solution or antihistamines but no chimeric binding agent.

### Example 14

### In Vivo Evaluation of Chimeric Binding Agents in Mouse Models of Allergic Airway Inflammation

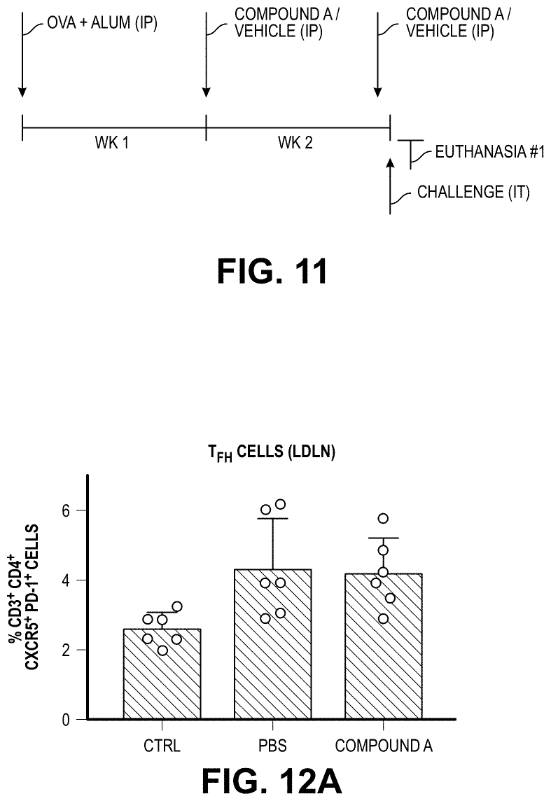

This example demonstrates the in vivo evaluation of a chimeric binding agent in a mouse model of allergic airway inflammation (FIG. 11).

The in vivo study described herein used C57BL/6J wild-type mice with 6 animals per cohort. Sensitization of animals at the beginning of the study (t=0, day 1) was performed using ovalbumin (OVA) emulsified in aluminum hydroxide (alum) at a mass ratio of 1:4. The OVA-alum was then administered via intraperitoneal (i.p.) injection at a dose of 1 mg/kg. Animals were then treated two times at days 8 and 14, respectively, by administering (i.p.) either vehicle or the chimeric binding agent which amino acid sequence is set forth in SEQ ID NO: 12 at a dose of 5 mg/kg. At day 15 of the study, the mice were challenged with antigen (OVA) administered intratracheally (i.t.) using a dose of 0.1 mg/kg. Animals were then euthanized for analysis at day 16. Study readouts included (i) germinal center reactions by evaluating T follicular helper cells and germinal center B cells in lung draining lymph nodes (LdLNs), (ii) effector T cells such as T_{H}2 cells in the lungs, (iii) serum or tissue levels of other effector cells such as mast cells, basophils, eosinophils and neutrophils, (iv) antibody responses such as total serum IgE and OVA-specific IgE levels, and (v) immediate responses such as serum histamine levels and IL-4 levels in the lungs.

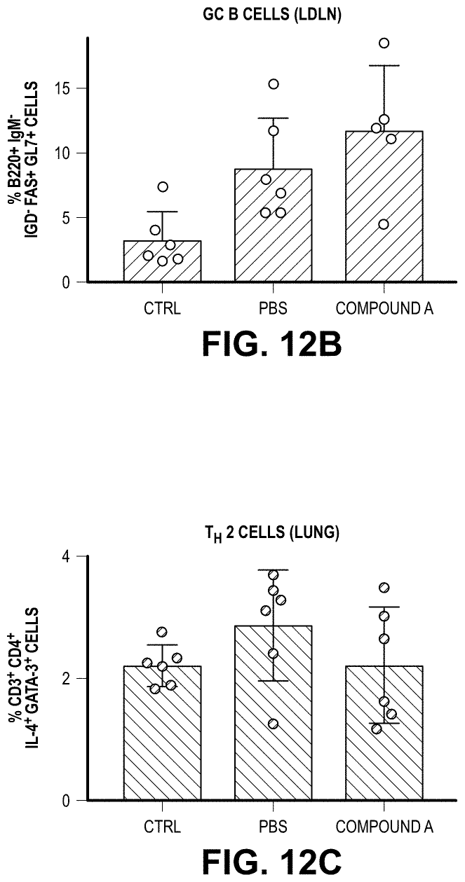

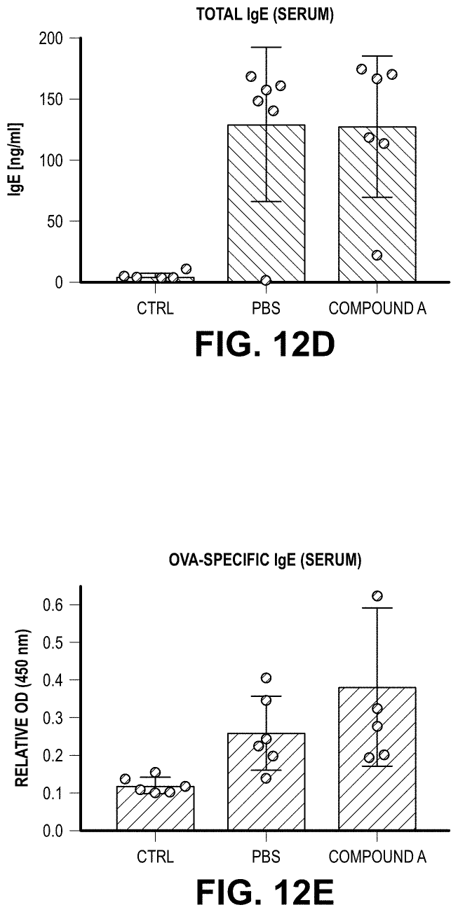

In the first part of this study it was investigated whether the chimeric binding agent (abbreviated herein and in the corresponding figures with “SEQ ID NO: 12”) interferes with antibody responses to immunization. FIGS. 12A-12E demonstrate that the chimeric binding agent did not interfere with antibody responses to immunization, as it did not significantly impact the levels of T follicular helper cells and germinal center B cells in LdLNs and T effector cells in lung tissue. This finding was further supported by the fact that total serum IgE and OVA-specific IgE levels were not impacted by administration of the chimeric binding agent. Specifically, FIGS. 12A-12E show serum levels of various T cells, B cells, as well as serum IgE levels of C57BL/6J mice from the following three different study cohorts: (i) mice that received no treatment and were immunized with PBS (CTRL cohort); (ii) mice that were immunized with OVA-alum and received vehicle PBS via i.p. injection (PBS cohort); or (iii) mice that were OVA-alum immunized and received the chimeric binding agent consisting of the amino acid sequence set forth in SEQ ID NO: 12 (5 mg/kg) via i.p. injection (compound A cohort). FIG. 12A shows the percentage of serum T follicular helper cells that stained positive for the markers CD3, CD4, CXCR5, and PD-1 in the three different cohorts of C57BL/6J mice and indicates that administration of the chimeric binding agent had no significant effect on the serum levels of these markers. FIG. 12B shows the percentage of serum germinal center B cells that stained positive for the markers B220, Fas, GL7 and negative for IgM and IgD in the three different cohorts of C57BL/6J mice and indicates that administration of the chimeric binding agent had no significant effect on the serum levels of these markers. FIG. 12C shows the percentage of serum T helper cells that stained positive for the markers CD3, CD4, IL-4, and GATA-3 in the three different cohorts of C57BL/6J mice and indicates that administration of the chimeric binding agent had no significant effect on the serum levels of these markers. FIG. 12D shows the total serum IgE concentration in the three different cohorts of C57BL/6J mice and indicates that administration of the chimeric binding agent had no significant effect on the serum IgE levels compared to administration of PBS vehicle. FIG. 12E further shows OVA-specific IgE serum levels given as relative optical density (OD, at 450 nm) in the three different cohorts of C57BL/6J mice and indicates that administration of the chimeric binding agent had no significant effect on the serum OVA-specific IgE levels compared to administration of PBS vehicle.

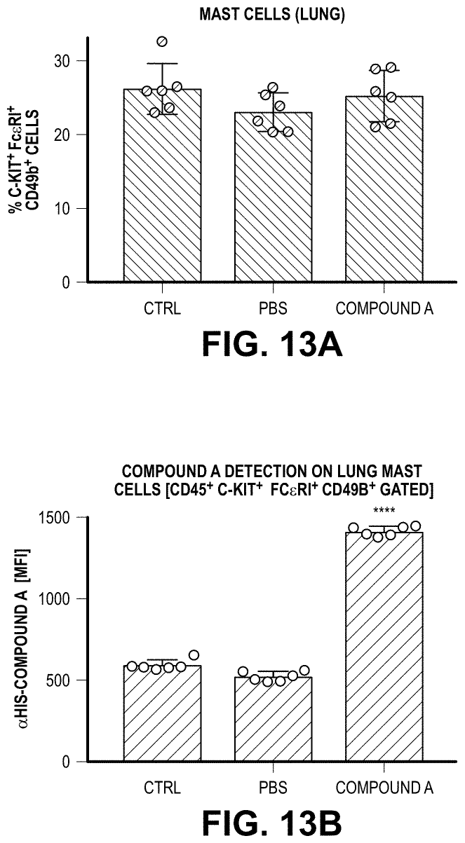

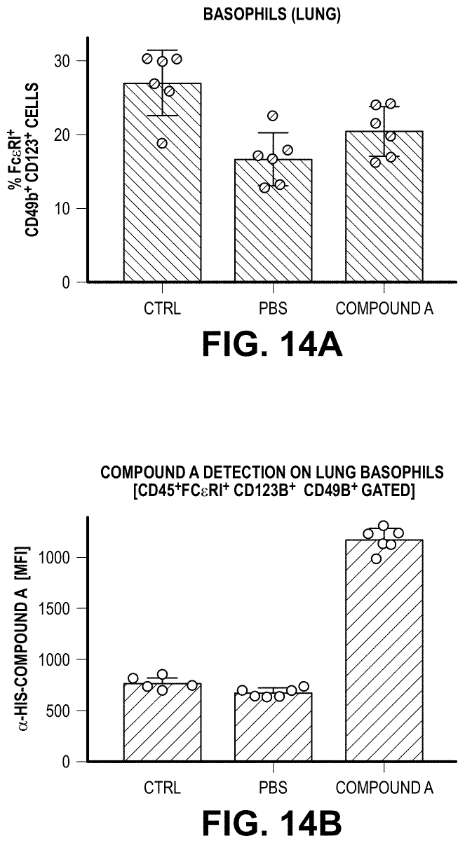

Next, it was demonstrated that the chimeric binding agent homes in on and binds to target cells such as mast cells. For example, FIGS. 13A-13B show that the chimeric binding agent containing the amino acid sequence set forth in SEQ ID NO: 12 was detectable on FcεRI-positive mast cells obtained from the three different “CTRL”, “PBS” and “SEQ ID NO: 12” C57BL/6J mouse cohorts described above in this example. FIG. 13A shows the percentages of mast cells that stained positive for the markers c-kit, FcεRI, and CD49b in serum of C57BL/6J mice from three different cohorts with mean values of 25.8 (CTRL cohort), 25.3 (PBS cohort), and 28.8 (compound A cohort), respectively. FIG. 13B shows the detection of α-His-SEQ ID NO: 12 (His-tagged) binding agent shown as mean fluorescence intensity on lung mast cells of C57BL/6J mice from three different cohorts. Flow cytometry experiments were CD45⁺ c-kit⁺ FcεRI⁺ CD49b⁺ gated. These data demonstrate that the chimeric binding agents was readily detected on FcεRI⁺ mast cells, another target cell population, in vivo. Furthermore, that data in FIG. 14A show the percentage of basophils that stained positive for the markers, FcεRI, CD49b, and CD123 in serum of C57BL/6J mice from three different cohorts with mean values of 5.06 (CTRL cohort), 3.51 (PBS cohort), and 4.23 (compound A cohort), respectively. FIG. 14B shows detection of α-His-SEQ ID NO: 12 (His-tagged) binding agent shown as mean fluorescence intensity on lung basophils of C57BL/6J mice from the three different cohorts. Flow cytometry experiments were CD45⁺ Fcε RI⁺ CD49b⁺ CD123⁺ gated. These data demonstrate that the chimeric binding agents was readily detected on Fcε Itt basophils, another target cell population, in vivo.

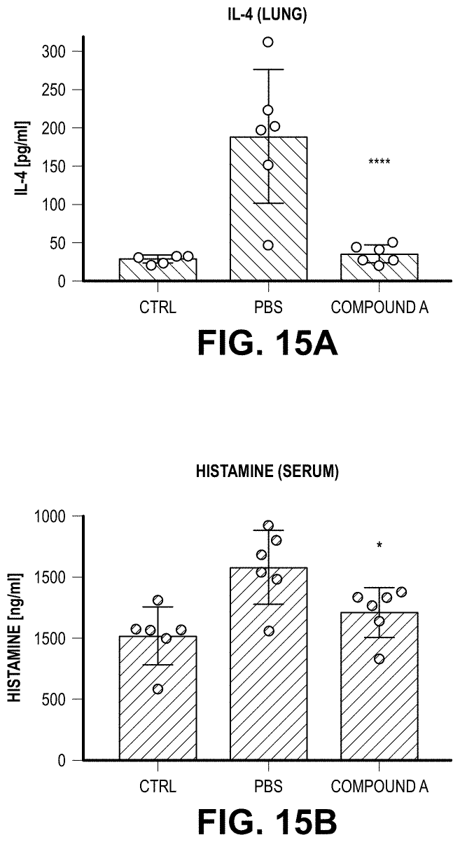

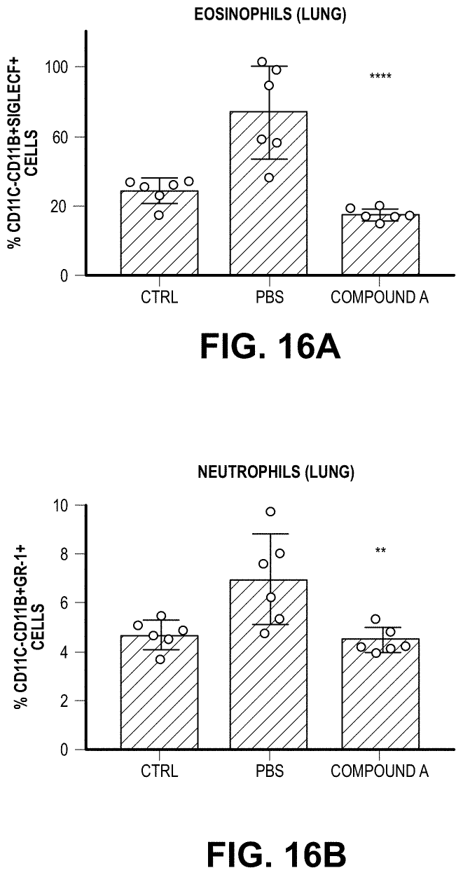

Upon demonstrating that the chimeric binding agent did not interfere with endogenous antibody responses to immunization and that it homes in on target cell populations including mast cells and basophils, it was investigated whether the binding agent is capable of preventing immediate responses to allergen challenge after the animals had been sensitized to the antigen. To that end, FIGS. 15A-15B show that in animals that received treatment with the chimeric binding agent, lung IL-4 and serum histamine levels, which were used as local and systemic inflammation markers, respectively, were significantly reduced compared to those animals in the PBS cohort that did not receive the chimeric binding agent prior to antigen challenge. Specifically, FIGS. 15A-15B show that the chimeric binding agent consisting of the amino acid sequence set forth in SEQ ID NO: 12 prevented immediate responses to allergen challenge in vivo as shown in the three different “CTRL”, “PBS” and “SEQ ID NO: 12” C57BL/6J mouse cohorts. FIG. 15A shows IL-4 concentrations (in pg/mL) in lung tissue from the three different cohorts. FIG. 15B shows serum histamine concentrations (in pg/mL) in the three different cohorts. These data demonstrate significant reductions in lung IL-4 and serum histamine levels in animals that were treated with the chimeric binding agent. In a follow-on experiment, it was shown that the chimeric binding agent also prevented the recruitment of eosinophils and neutrophils following antigen challenge, which further demonstrates that the chimeric binding agents of the present disclosure can effectively prevent immune response to allergen challenges in vivo. Specifically, FIGS. 16A-16B show that the chimeric binding agent consisting of the amino acid sequence set forth in SEQ ID NO: 12 prevented recruitment of eosinophils and neutrophils to the lungs, as shown in the three different “CTRL”, “PBS” and “SEQ ID NO: 12” C57BL/6J mouse cohorts. FIG. 16A shows the percentage of lung eosinophils that stained positive for the markers CD11b and SiglecF and negative for CD11c in serum of C57BL/6J mice from three different cohorts with mean values of 1.28 (CTRL cohort), 3.07 (PBS cohort), and 1.01 (compound A cohort), respectively. FIG. 16B shows the percentages of lung neutrophils that stained positive for the markers CD11b and Gr-1 and negative for Cd11c in serum of C57BL/6J mice from three different cohorts with mean values of 3.80 (CTRL cohort), 9.77 (PBS cohort), and 4.23 (compound A cohort), respectively. These data show a significant reduction in eosinophil and neutrophil recruitment in animals that were treated with the chimeric binding agent.

Taken together, the results of this in vivo study demonstrate that the chimeric binding agents of the present disclosure can effectively prevent and treat immune responses to allergen challenges in vivo.

### Example 15

### In Vivo Evaluation of an IgE-Fc-IgG-Fc Chimeric Binding Agent

This example evaluates whether a chimeric binding comprising an IgE-Fc region linked via its C-terminus to an IgG-Fc region can provide anti-inflammatory activity in vivo with a low immunogenicity.

Immune competent mice are treated with different doses of the chimeric binding agent IgE-Fc-IgG-Fc ranging from 2.5 mg/kg to 10 mg/kg at various time points before antigen challenge in order to investigate the pharmacokinetic of the IgE-Fc-IgG-Fc fusion proteins and the pharmacodynamic of FcR interaction and potential effective receptor blockage. The constructs in this example comprise an IgE-Fc region linked via its C-terminus to an IgG-Fc region via the IgG-Fc hinge region as a linker. The constructs that can be used in this example contain the amino acid sequence set forth in SEQ ID NO: 21 or 23.

### Example 16

### Production of a Trivalent Chimeric Binding Agent

This example shows the production of a trivalent chimeric binding agent. The trivalent chimeric binding agent of this example contained two human IgG1-Fc regions, both comprising a “SELF” substitution, that were C-terminally fused via the (G₄S)₃ linker (SEQ ID NO: 14) to a human IgE-Fc region (see e.g., FIG. 1G). The two IgG-Fc regions comprised the amino acid sequence set forth in SEQ ID NO: 26, whereas the IgE-Fc region comprised the amino acid sequence set forth in SEQ ID NO: 29.

The IgG-Fc regions contained Knob-into-Hole mutations. Specifically, the IgG-Fc region that was fused via the linker to an IgE-Fc region (construct having the amino acid sequence set forth in SEQ ID NO: 25) contained the “Hole” mutation, whereas a separately expressed open reading frame coding for the second IgG-Fc region contained the “Knob” mutation. Both open reading frames were cloned onto a single vector with two promoter regions (the vector having the nucleotide sequence set forth in SEQ ID NO: 24), which enabled simultaneous expression of both the IgG-Fc-(G₄S)₃-IgE-Fc construct and the second, shorter IgG-Fc protein. During translation, both proteins were co-translationally translocated into the ER, where protein folding and chain assembly took place. During this process, two IgG-Fc-Linker-IgE-Fc align with each other, but chain pairing only occurred in the IgE Fc region. The introduced “Hole” mutations in the IgG-Fc region prevented the two chains from pairing with each other. Simultaneous expression of the shorter, second IgG-Fc carrying the “Knob” mutation ensured that only IgG-Fc (Hole) paired with IgG-Fc (Knob) fragments.

The result of this targeted assembly was a trivalent Fc fusion protein, consisting of one IgE Fc and two IgG1 Fc domains.

### Example 17

### In Vitro and In Vivo Evaluation of a Trivalent Chimeric Binding Agent

This example shows the in vivo evaluation of the trivalent chimeric binding agent produced in EXAMPLE 16.

In vitro experiments show that binding affinity of the IgE-Fc portion to FceRIs remains unchanged, which allows the targeting of FceRI+ target cells such as mast cells and basophils, thereby blocking the accessibility of these receptors for allergen-specific IgE. In case of the 2 IgG-Fc portions, these two IgG-Fc portions do not affect their respective binding affinity to FcγRIIBs. However, it can be observed that the presence of 2 IgG-Fc regions in the construct can significantly increase the avidity of the trivalent construct compared to bivalent constructs.

In vivo, it is evaluated whether an increase in avidity can significantly increase the duration of the trivalent binding agent being bound to the target cells. Moreover, it is evaluated in vivo whether the ability of the trivalent construct to engage twice as many inhibitory receptors FcγRIIB, and co-ligate them with FceIRs, can induce an even more potent suppression of effector cells through the ITIM mediated pathway that results in cell inactivation.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A chimeric binding agent, wherein the chimeric binding agent comprises:
(i) an IgE-Fc region; and
(ii) a plurality of IgG-Fc regions,
wherein the IgE-Fc region binds to an Fc epsilon (Fcε) receptor and the plurality of IgG-Fc regions binds to an Fc gamma (Fcγ) receptor, thereby inhibiting the Fcε receptor while activating the Fcγ receptor.

2. The chimeric binding agent of claim 1, wherein inhibiting the Fcε receptor and activating the Fcγ receptor occurs substantially simultaneously.

3. The chimeric binding agent of claim 1, wherein the FcεR is an FcεRI.

4. The chimeric binding agent of claim 1, wherein the FcγR is an FcγRIIB.

5. The chimeric binding agent of claim 1, wherein the IgE-Fc region binds to the Fcε receptor with an association constant (Kₐ) from about 10⁸M⁻¹ to about 10¹² M⁻¹.

6. The chimeric binding agent of claim 1, wherein the IgE-Fc region further binds to a CD23 receptor.

7. The chimeric binding agent of claim 6, wherein the IgE-Fc region binds to the CD23 receptor with an association constant (Kₐ) from about 10⁶ M⁻¹ to about 10¹⁰ M⁻¹.

8. The chimeric binding agent of claim 1, wherein the IgE-Fc region is linked to the plurality of IgG Fc regions.

9. The chimeric binding agent of claim 1, wherein a C-terminus of the IgE-Fc region is linked to an N-terminus of an IgG-Fc region of the plurality of IgG-Fc regions.

10. The chimeric binding agent of claim 1, wherein an N-terminus of the IgE-Fc region is linked to a C-terminus of an IgG Fc region of the plurality of IgG-Fc regions.

11. The chimeric binding agent of claim 8, wherein the IgE-Fc region is directly linked to the plurality of IgG Fc regions.

12. The chimeric binding agent of claim 8, wherein the IgE-Fc region is covalently and directly linked to the plurality of IgG Fc regions.

13. The chimeric binding agent of claim 8, wherein the IgE-Fc region is linked to the plurality of IgG Fc regions via a linker.

14. The chimeric binding agent of claim 13, wherein the linker comprises one or more amino acid residues.

15. The chimeric binding agent of claim 14, wherein the linker comprises the amino acid sequence set forth in SEQ ID NO: 13 or SEQ ID NO: 14.

16. The chimeric binding agent of claim 13, wherein the linker comprises a hinge region of an immunoglobulin molecule.

17. The chimeric binding agent of claim 16, wherein the hinge region is from an IgG-Fc region.

18. The chimeric binding agent of claim 1, wherein the IgE-Fc region comprises a Cε2 domain, a Cε3 domain, a Cε4 domain, or a combination thereof.

19. The chimeric binding agent of claim 1, wherein an IgG-Fc region of the plurality of IgG Fc regions comprises a Cγ2 domain, a Cγ3 domain, or a combination thereof.

20. The chimeric binding agent of claim 1, wherein the IgE-Fc region comprises a Cε2 domain, a Cε3 domain, and a Cε4 domain, and an IgG-Fc region of the plurality of IgG Fc regions comprises a Cγ2 domain and a Cγ3 domain.

21. The chimeric binding agent of claim 20, wherein the IgG-Fc region of the plurality of IgG Fc regions is from an Immunoglobulin G1 (IgG1), IgG2, IgG3, or IgG4 molecule.

22. The chimeric binding agent of claim 20, wherein the IgG-Fc region of the plurality of IgG Fc regions is from an IgG1 molecule.

23. The chimeric binding agent of claim 1, wherein the IgE-Fc region is a human IgE-Fc region.

24. The chimeric binding agent of claim 23, wherein the human IgE-Fc region is a human wild-type IgE-Fc region.

25. The chimeric binding agent of claim 23, wherein the human IgE-Fc region comprises at least one amino acid substitution, addition, and/or deletion, or a combination thereof.

26. The chimeric binding agent of claim 25, wherein the at least one amino acid substitution comprises a T396F substitution.

27. The chimeric binding agent of claim 1, wherein at least one IgG-Fc region of the plurality of IgG-Fc regions is a human IgG-Fc region.

28. The chimeric binding agent of claim 27, wherein the at least one human IgG-Fc region of the plurality of IgG-Fc regions is a human wild-type IgG-Fc region.

29. The chimeric binding agent of claim 27, wherein the at least one human IgG-Fc region comprises at least one amino acid substitution, addition, and/or deletion, or a combination thereof.

30. The chimeric binding agent of claim 29, wherein the at least one amino acid substitution comprises a S267E, L328F, or an E333A substitution, or a combination thereof.

31. The chimeric binding agent of claim 29, wherein the at least one amino acid substitution comprises a S267E and an L328F substitution.

32. The chimeric binding agent of claim 1, wherein the plurality of IgG-Fc regions is capable of antibody-dependent cell-mediated cytotoxicity (ADCC)-mediated mast cell or basophil depletion.

33. The chimeric binding agent of claim 32, wherein at least one IgG-Fc region of the plurality of IgG-Fc regions comprises a glutamic acid (E) to alanine (A) substitution relative to a human wild-type IgG-Fc region.

34. The chimeric binding agent of claim 33, wherein the glutamic acid (E) to alanine (A) substitution is an E333A substitution.

35. The chimeric binding agent of claim 1, wherein the plurality of IgG-Fc regions consists of two IgG-Fc regions.

36. The chimeric binding agent of claim 1, wherein the plurality of IgG-Fc regions consists of three, four, or five IgG-Fc regions.

37. The chimeric binding agent of claim 1, wherein the IgE-Fc region comprises an amino acid sequence having at least 80%, 85%, 90%, 95%, 97%, or 99% sequence identity or similarity to the amino acid sequence set forth in any one of SEQ ID NOs: 3-6, or 29.

38. The chimeric binding agent of claim 1, wherein the IgE-Fc region comprises an amino acid sequence having at least 80%, 85%, 90%, 95%, 97%, or 99% sequence identity or similarity to the amino acid sequence set forth in SEQ ID NO: 29.

39. The chimeric binding agent of claim 1, wherein the IgE-Fc region comprises the amino acid sequence set forth in SEQ ID NO: 29.

40. The chimeric binding agent of claim 1, wherein an IgG-Fc region of the plurality of IgG-Fc regions comprises an amino acid sequence having at least 80%, 85%, 90%, 95%, 97%, or 99% sequence identity or similarity to the amino acid sequence set forth in any one of SEQ ID NOs: 9-10, or 26.

41. The chimeric binding agent of claim 1, wherein the IgG-Fc region comprises an amino acid sequence having at least 80%, 85%, 90%, 95%, 97%, or 99% sequence identity or similarity to the amino acid sequence set forth in SEQ ID NO: 10.

42. The chimeric binding agent of claim 1, wherein the IgG-Fc region comprises an amino acid sequence having at least 80%, 85%, 90%, 95%, 97%, or 99% sequence identity or similarity to the amino acid sequence set forth in SEQ ID NO: 26.

43. A chimeric binding agent, wherein the chimeric binding agent comprises:
(i) an IgE-Fc region, wherein the IgE-Fc region comprises a Cε2 domain, a Cε3 domain, a Cε4 domain, or a combination thereof; and
(ii) at least one IgG-Fc region, wherein the at least one IgG-Fc region comprises a Cγ2 domain, a Cγ3 domain, or a combination thereof;
wherein a C-terminus of the IgE-Fc region is linked to an N-terminus of the at least one IgG-Fc region.

44. The chimeric binding agent of claim 43, wherein the IgE-Fc region is linked to the at least one IgG-Fc region.

45. The chimeric binding agent of claim 44, wherein the IgE-Fc region is directly linked to the at least one IgG-Fc region.

46. The chimeric binding agent of claim 44, wherein the IgE-Fc region is covalently and directly linked to the at least one IgG-Fc region.

47. The chimeric binding agent of claim 44, wherein the IgE-Fc region is linked to the at least one IgG-Fc region via a linker.

48. The chimeric binding agent of claim 47, wherein the linker comprises one or more amino acid residues.

49. The chimeric binding agent of claim 48, wherein the linker comprises the amino acid sequence set forth in SEQ ID NO: 13 or SEQ ID NO: 14.

50. The chimeric binding agent of claim 48, wherein the linker comprises a hinge region of an immunoglobulin molecule.

51. The chimeric binding agent of claim 50, wherein the hinge region is from the at least one IgG-Fc region.

52. The chimeric binding agent of claim 43, wherein the IgE-Fc region binds to an FcεR.

53. The chimeric binding agent of claim 52, wherein the FcεR is an FcεRI.

54. The chimeric binding agent of claim 43, wherein the at least one IgG-Fc region binds to an FcγR.

55. The chimeric binding agent of claim 54, wherein the FcγR is an FcγRIIB.

56. The chimeric binding agent of claim 52, wherein the IgE-Fc region binds to the Fcε receptor with an association constant (Kₐ) from about 10⁸ M⁻¹ to about 10¹² M⁻¹.

57. The chimeric binding agent of claim 52, wherein the IgE-Fc region further binds to a CD23 receptor.

58. The chimeric binding agent of claim 57, wherein the IgE-Fc region binds to the CD23 receptor with an association constant (Kₐ) from about 10⁶ M⁻¹ to about 10¹⁰ M⁻¹.

59. The chimeric binding agent of claim 43, wherein the IgE-Fc region comprises a Cε2 domain, a Cε3 domain, and a Cε4 domain.

60. The chimeric binding agent of claim 43, wherein the at least one IgG-Fc region comprises a Cγ2 domain and a Cγ3 domain.

61. The chimeric binding agent of claim 43, wherein the at least one IgG-Fc region is from an Immunoglobulin G1 (IgG1), IgG2, IgG3, or IgG4 molecule.

62. The chimeric binding agent of claim 43, wherein the at least one IgG-Fc region is from an IgG1 molecule.

63. The chimeric binding agent of claim 43, wherein the IgE-Fc region is a human IgE-Fc region.

64. The chimeric binding agent of claim 63, wherein the human IgE-Fc region is a human wild-type IgE-Fc region.

65. The chimeric binding agent of claim 64, wherein the human IgE-Fc region comprises at least one amino acid substitution, addition, and/or deletion, or a combination thereof.

66. The chimeric binding agent of claim 65, wherein the at least one amino acid substitution comprises a T396F substitution.

67. The chimeric binding agent of claim 43, wherein the at least one IgG-Fc region is a human IgG-Fc region.

68. The chimeric binding agent of claim 67, wherein the human IgG-Fc region is a human wild-type IgG-Fc region.

69. The chimeric binding agent of claim 67, wherein the human IgG-Fc region comprises at least one amino acid substitution, addition, and/or deletion, or a combination thereof.

70. The chimeric binding agent of claim 69, wherein the at least one amino acid substitution comprises a S267E, L328F, and/or E333A substitution, or a combination thereof.

71. The chimeric binding agent of claim 69, wherein the human IgG-Fc region comprises a S267E and an L328F substitution.

72. The chimeric binding agent of claim 43, wherein the at least one IgG-Fc region is capable of antibody-dependent cell-mediated cytotoxicity (ADCC)-mediated mast cell or basophil depletion.

73. The chimeric binding agent of claim 72, wherein the at least one IgG-Fc region comprises a glutamic acid (E) to alanine (A) substitution relative to a human wild-type IgG-Fc region.

74. The chimeric binding agent of claim 73, wherein the glutamic acid (E) to alanine (A) substitution is an E333A substitution.

75. The chimeric binding agent of claim 43, wherein the at least one IgG-Fc region consists of one IgG-Fc region.

76. The chimeric binding agent of claim 43, wherein the at least one IgG-Fc region consists of two IgG-Fc regions.

77. The chimeric binding agent of claim 43, wherein the at least one IgG-Fc region consists of three, four, or five IgG-Fc regions.

78. The chimeric binding agent of claim 43, wherein the IgE-Fc region comprises an amino acid sequence having at least 80%, 85%, 90%, 95%, 97%, or 99% sequence identity or similarity to the amino acid sequence set forth in any one of SEQ ID NOs: 3-6.

79. The chimeric binding agent of claim 43, wherein the IgE-Fc region comprises an amino acid sequence having at least 80%, 85%, 90%, 95%, 97%, or 99% sequence identity or similarity to the amino acid sequence set forth in SEQ ID NO: 6.

80. The chimeric binding agent of claim 43, wherein the IgE-Fc region comprises the amino acid sequence set forth in SEQ ID NO: 6.

81. The chimeric binding agent of claim 43, wherein an IgG-Fc region of the plurality of IgG-Fc regions comprises an amino acid sequence having at least 80%, 85%, 90%, 95%, 97%, or 99% sequence identity or similarity to the amino acid sequence set forth in any one of SEQ ID NOs: 9-10, or 26.

82. The chimeric binding agent of claim 43, wherein the IgG-Fc region comprises an amino acid sequence having at least 80%, 85%, 90%, 95%, 97%, or 99% sequence identity or similarity to the amino acid sequence set forth in SEQ ID NO: 10.

83. The chimeric binding agent of claim 43, wherein the IgG-Fc region comprises an amino acid sequence having at least 80%, 85%, 90%, 95%, 97%, or 99% sequence identity or similarity to the amino acid sequence set forth in SEQ ID NO: 26.

84. The chimeric binding agent of claim 43, wherein the chimeric binding agent comprises an amino acid sequence having at least 80%, 85%, 90%, 95%, 97%, or 99% sequence identity or similarity to the amino acid sequence set forth in SEQ ID NO: 21.

85. The chimeric binding agent of claim 43, wherein the chimeric binding agent comprises an amino acid sequence having at least 80%, 85%, 90%, 95%, 97%, or 99% sequence identity or similarity to the amino acid sequence set forth in SEQ ID NO: 23.

86. The chimeric binding agent of claim 43, wherein the chimeric binding agent comprises the amino acid sequence set forth in SEQ ID NO: 21.

87. The chimeric binding agent of claim 43, wherein the chimeric binding agent comprises the amino acid sequence set forth in SEQ ID NO: 23.

88. A chimeric binding agent comprising an amino acid sequence having at least 80%, 85%, 90%, 95%, 97%, or 99% sequence identity or similarity to the amino acid sequence set forth in any one of SEQ ID NOs: 12, 25, or 28.

89. The chimeric binding agent of claim 88, wherein the chimeric binding agent comprises an amino acid sequence having at least 80%, 85%, 90%, 95%, 97%, or 99% sequence identity to the amino acid sequence set forth in any one of SEQ ID NOs: 12, 25, or 28.

90. The chimeric binding agent of claim 88, wherein the chimeric binding agent comprises an amino acid sequence having at least 80%, 85%, 90%, 95%, 97%, or 99% sequence identity to the amino acid sequence set forth in SEQ ID NO: 12.

91. The chimeric binding agent of claim 88, wherein the chimeric binding agent comprises an amino acid sequence having at least 80%, 85%, 90%, 95%, 97%, or 99% sequence identity to the amino acid sequence set forth in SEQ ID NO: 25.

92. The chimeric binding agent of claim 88, wherein the chimeric binding agent comprises an amino acid sequence having at least 80%, 85%, 90%, 95%, 97%, or 99% sequence identity to the amino acid sequence set forth in SEQ ID NO: 28.

93. The chimeric binding agent of claim 88, wherein the chimeric binding agent comprises the amino acid sequence set forth in SEQ ID NO: 12.

94. The chimeric binding agent of claim 88, wherein the chimeric binding agent comprises the amino acid sequence set forth in SEQ ID NO: 25.

95. The chimeric binding agent of claim 88, wherein the chimeric binding agent comprises the amino acid sequence set forth in SEQ ID NO: 28.

96. A pharmaceutical composition comprising the chimeric binding agent of any one of claims 1-95 and one or more pharmaceutically acceptable excipients.

97. The pharmaceutical composition of claim 96, wherein the chimeric binding agent is conjugated to a vehicle.

98. The pharmaceutical composition of claim 97, wherein the vehicle is a protein-based vehicle or a lipid-based vehicle.

99. The pharmaceutical composition of claim 96, wherein the pharmaceutical composition is a subcutaneous dosage form.

100. The pharmaceutical composition of claim 96, wherein the pharmaceutical composition is an intravenous dosage form.

101. The pharmaceutical composition of claim 96, wherein the pharmaceutical composition is an oral dosage form.

102. The pharmaceutical composition of claim 96, wherein an amount of the chimeric binding agent in the pharmaceutical composition is from about 0.01 mg/kg to about 10 mg/kg by weight of a subject.

103. The pharmaceutical composition of claim 102, wherein the subject is a rodent or a human.

104. A method of treating or preventing inflammation in a subject in need thereof, the method comprising administering to the subject a pharmaceutical composition of any one of claims 96-103, wherein the pharmaceutical composition is present in an effective amount for treating or preventing the inflammation in the subject.

105. The method of claim 104, further comprising administering a bacterial consortium to the subject.

106. The method of claim 105, wherein the bacterial consortium comprises Lactobacillus sp., Faecalibacterium sp., or Akkermansia sp., or a combination thereof.

107. The method of claim 104, wherein the inflammation is an allergic inflammation.

108. The method of claim 107, wherein the allergic inflammation is allergic asthma or allergic airway inflammation.

109. The method of claim 104, wherein the inflammation is an IgE-mediated inflammatory disease.

110. The method of claim 109, wherein the IgE-mediated inflammatory disease is Hyper-IgE-Syndrome.

111. The method of claim 104, wherein the subject is a mammal.

112. The method of claim 104, wherein the subject is a rodent or a human.

113. A method of depleting FceRI+ cells in a cell population, the method comprising contacting the cell population with a composition of any one of claims 1-95, wherein the composition depletes the FceRI+ cells in the cell population via antibody-dependent cell-mediated cytotoxicity (ADCC), and wherein depleting the FceRI+ cells in the cell population comprises depleting at least 30% of FceRI+ cells in the cell population after contacting the cell population with the composition for 12 hours.

114. The method of claim 113, wherein the composition is of any one of claim 31-33, 72-74, or 88-95.

115. The method of claim 113, wherein the cell population is from a mammal.

116. The method of claim 113, wherein the cell population is from a rodent or a human.

117. The method of claim 113, wherein the composition depletes at least 50% of FceRI+ cells in the cell population after 6 hours.
